(19) United States
(12) Patent Application Publication
(10) Pub. No.: US 2005/0043354 A1
(43) Pub. Date: Feb. 24, 2005

(54) NK1 antagonist

(75) Inventors: Travis T. Wager, New London, CT (US); Willard M. Welch, Mystic, CT (US); Brian T. O'Neill, Old Saybrook, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/868,919

(22) Filed: Jun. 15, 2004

Related U.S. Application Data
(60) Provisional application No. 60/479,901, filed on Jun. 19, 2003.

(51) Int. Cl. C07D 41/02; A61K 31/445; C07D 211/54

(52) U.S. Cl. 514/317; 546/226; 514/326; 546/207; 546/216

(57) ABSTRACT

The invention is to a compound exhibiting neurokinin inhibitory properties, a pharmaceutical composition comprising same and a method of treatment for neurokinin-mediated conditions.

### BACKGROUND OF THE INVENTION

1. Field of the Invention

The invention pertains to a compound which is an antagonist to tachykinins, including substance P and other neurokinins (NK); to a pharmaceutical composition comprising same; and a method of treating of neurokinin-mediated diseases, among others.

2. Description of the Prior Art

Substance P (also known as NK-1) is a naturally occurring undecapeptide belonging to the tachykinin family of peptides, so named due to their prompt stimulatory action on smooth muscle tissue. More specifically, substance P is a pharmacologically active neuropeptide produced in mammals and possessing a characteristic amino acid sequence as illustrated in U.S. Pat. No. 4,680,283.

### SUMMARY OF THE INVENTION

The invention relates to a compound having Formula I: 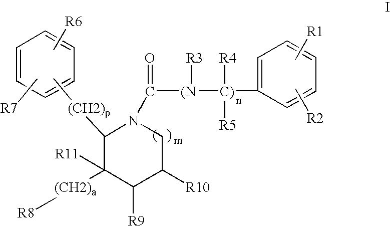
or pharmaceutically acceptable salts and solvates thereof, the (R) and (S) enantiomers thereof and the cis and trans isomers thereof
wherein
m=0 or 1; n=0 or 1; p=0, 1, 2 or 3; a=0, 1, 2 or 3;
   R1 and R2 are each independently C₁₋₆alkyl, C₁₋₆alkoxy, —CF₃, —OCF₃, or halogen;
   R3 is hydrogen or C₁₋₆alkyl;
   R4 is hydrogen, C₁₋₆alkyl, C₂₋₆alkenyl, C₃₋₇cycloalkyl, or R4 and R3 together with the C and N atoms to which they are respectively attached form a 5 to 6 member heterocyclic group;
   R5 is hydrogen, C₁₋₆alkyl, or R5 and R4 together with the C atom to which they are attached form a C₃₋₇cycloalkyl;
   R6 and R7 are each independently hydrogen, halogen or C₁₋₆alkyl;
   R9 and R10 are each independently hydrogen, C₁₋₆alkyl or, when m=1, R10 and R8 together with R9 and the C atoms to which they are respectively attached, may form a 8 to 14 member heterobicyclic ring which heterobicyclic ring may be optionally substituted with one or more C₁₋₆alkyl or C₅₋₁₀aryl;
   R11 is hydrogen or R11 and R9 together with the C atoms to which they are respectively attached form a C₃₋₇cycloalkyl or when M=0 and R10 is hydrogen, R9 and R11 together with the C atom to which they are attached form a 5 to 7 member heterocyclic ring;
   R8 is:
      i) hydrogen, a C₁₋₆alkyl group, or a C₁₋₇acyl group, either of which groups may be optionally substituted with one or more hydroxy, amino, C₁₋₆alkoxy or substituted with a 4 to 8 member heterocyclic ring or a 5 to 7 member heteroaryl ring either of which rings may be optionally substituted with one or more C₁₋₄alkyl, amino, hydroxy, C₁₋₆alkoxy or C₁₋₇acyl; 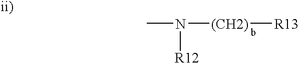
      wherein b=0, 1, 2 or 3 and R12 and R13 are each independently hydrogen or one of the following groups: C₁₋₆alkyl, C₃₋₇cycloalkyl, C₁₋₆alkoxycarbonyl, C₅₋₁₀aryl, C₁₋₆alkoxy, C₁₋₇acyl, amino, amido, C₁₋₇acylamino, a 4 to 8 member heterocyclic ring, a 5 to 7 member heteroaryl ring or a C₆₋₁₄heterobicyclic ring, any one of which groups may be optionally substituted with one or more hydroxy, halogen, oxo, C₁₋₇acyl, amino, morpholino or C₁₋₄alkyl; 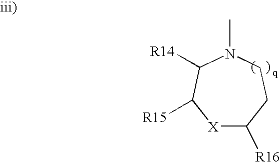
         wherein q=0 or 1
         R14, R15 and R16 are each independently hydrogen, C₁₋₄alkyl or oxo;
         X is O, S or NR17, wherein R17 is hydrogen, C₁₋₆alkyl, C₃₋₇cycloalkyl, C₅₋₁₀aryl, C₁₋₆alkoxycarbonyl, C₁₋₇acyl, amido, or 5 to 7 member heteroaryl ring, any one of which may be optionally substituted with one or more hydroxy, halogen, C₁₋₄alkyl or C₁₋₄alkoxy; or R17 and R15 together with the N and C atoms to which they are attached respectively form a 5 to 8 member heterocyclic ring or a 5 to 7 member heteroaryl ring, either of which rings may be optionally substituted with one or more hydroxy or C₁₋₄alkyl; 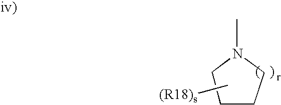
      wherein
         r=0, 1, 2, 3 or 4
         s=0, 1, 2 or 3
         each R¹⁸ is individually hydrogen, hydroxyl, C₅₋₁₀aryl, C₁₋₇acyl, amino, piperidinyl, oxadiazolyl, C₁₋₆alkoxy which alkoxy may be optionally substituted with an amido or C₁₋₆alkyl which alkyl may be optionally substituted with an alkoxy, amino, hydroxy or pyrrolyl group; or when m=0 R⁸ and R⁹ together with the C atoms to which they are attached may form a 5-member heterocyclic ring which heterocyclic ring may be optionally substituted with
            a) C₁₋₆alkyl which alkyl may be optionally substituted with C₅₋₁₀aryl, or
            b) a group of the formula 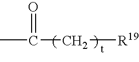
            wherein t=0, 1 or 2 and R¹⁹ is a 4 to 8 member heterocyclic ring.

### DETAILED DESCRIPTION OF THE INVENTION

The present invention relates to a compound (that in various practices comprises piperidine, pyrrolidine, diazepane derivatives) which is an antagonist of tachykinins, including substance P and other neurokinins (NK), such as NK1, and are thus useful for the treatment of neurokinin-mediated conditions, among other things.

In a preferred embodiment, the compound of the invention has Formula I, above, including pharmaceutically acceptable salts thereof, e.g. acid addition salts, base addition salts, and prodrugs and solvates thereof. Without limitation, examples of pharmaceutically acceptable acid addition salts of the compounds of Formula I are the salts of hydrochloric acid, p-toluenesulfonic acid, fumaric acid, citric acid, succinic acid, salicylic acid, oxalic acid, hydrobromic acid, phosphoric acid, methanesulfonic acid, tartaric acid, malate, di-p-toluoyl tartaric acid, and mandelic acid.

The compound of Formula I may have optical centers and thus occur in different enantiomeric configurations. The invention includes all enantiomers, diastereomers, and other stereoisomers and optical isomers of such compound of Formula I, as well as racemic and other mixtures thereof. For example, the compound of Formula I includes (R) and (S) enantiomers and cis and trans isomers. The present invention further includes all radiolabelled forms of the compound of the Formula I. Preferred radiolabelled compounds are those wherein the radiolabels are selected from as ³H, ¹¹C, ¹⁴C, ¹⁸F, ¹²³I and ¹²⁵I. Such radiolabelled compounds are useful as research and diagnostic tools in metabolism pharmacokinetics studies and in binding assays in animals and man.

As appreciated by the artisan, the use of Formula I is a convenience and the invention is understood to envision and embrace each and every species thereunder as though individually identified and set forth herein. Thus the present invention severally contemplates each species separately and any and all combinations and permutations of species falling within Formula I.

In a first preferred practice of the compound of Formula I, m=0, n=1, p=0, a=0 or 1; R1 and R2 are each —CF₃; R3 and R4 are each C₁₋₆alkyl; R5, R9, R10 and R11 are each hydrogen; R6 is C₁₋₆alkyl, R7 is halogen. In one embodiment of this practice, R3, R4 and R6 are each methyl; R7 is F; and R8 is (i). In another embodiment of this practice, a=0; R3, R4 and R6 are each methyl; R7 is F; and R8 is (ii). In a third embodiment of this practice, a=1, R3, R4 and R6 are each methyl; R7 is F; and R8 is (ii). In a fourth embodiment of this practice, a=1, R3, R4 and R6 are each methyl; R7 is F; and R8 is (iii). In a fifth embodiment of this practice, a=0, R9 and R11 together with the C atoms to which they are respectively attached form a 5 to 7 member heterocyclic ring; R5 and R10 are each hydrogen; R6 is methyl; R7 is F; R1 and R2 are each CF₃; R3 and R4 are each C₁₋₃alkyl and R8 is (i), preferably R8 is hydrogen.

In a second preferred practice, m=1, n=0, p=1, a=0 or 1; R1 and R2 are each —CF₃; R6, R7, R9, R10 and R11 are each hydrogen; and R8 is (ii) or (iii). In more preferred embodiments of this practice, when a=0, R8 is (ii); and a=1, R8 is (iii).

In a third preferred practice m=1, n=1, p=0 and a=1. In an embodiment of this practice, R8 and R10 together with R9 and the C atoms to which they are respectively attached form an 8 to 14 member heterobicyclic ring; in a more preferred aspect of this embodiment, R5, R9 and R11 are each hydrogen; R7 is methyl; R6 is F, R1 and R2 are each CF₃; and R3 and R4 are each C₁₋₃alkyl.

In another aspect, the compound of the invention is used in an assay of NK-1 binding wherein said compound exhibits a Ki of about 1 uM or less; preferably, said Ki is about 10 nM or less.

The present invention is also directed to a pharmaceutical composition comprising the compound of the invention; and a pharmaceutically acceptable carrier.

Unless otherwise indicated, the following terms and related variations of same as used herein representatively have the meanings ascribed:

“Halogen” and “halo” and the like includes fluoro, chloro, bromo and iodo.

“Alkyl” including as appears in the terms “alkoxy” and “alkyoxycarbonyl” includes saturated monovalent hydrocarbon radicals having straight or branched moieties. Examples of alkyl groups include, but are not limited to, methyl, ethyl, n-propyl, isopropyl, and t-butyl. “Alkoxycarbonyl” is —C(═O)—OR^{A} wherein R^{A} is C₁₋₆alkyl as defined herein.

“Cycloalkyl” includes non-aromatic saturated cyclic alkyl moieties wherein alkyl is as defined above. Examples of cycloalkyl include, but are not limited to, cyclopropyl, cyclobutyl, cyclopentyl, cyclohexyl, and cycloheptyl; and bicycloalkyl and tricycloalkyl groups that are non-aromatic saturated carbocyclic groups consisting of two or three rings respectively, wherein said rings share at least one carbon atom. For purposes of the present invention, and unless otherwise indicated, bicycloalkyl groups include spiro groups and fused ring groups. Examples of bicycloalkyl groups include, but are not limited to, bicyclo-[3.1.0]-hexyl, bicyclo-2.2.1]-hept-1-yl, norbornyl, spiro[4.5]decyl, spiro[4.4]nonyl, spiro[4.3]octyl, and spiro[4.2]heptyl. An example of a tricycloalkyl group is adamantanyl. Cycloalkyl groups also include groups that are substituted with one or more oxo moieties. Examples of such groups with oxo moieties are oxocyclopentyl and oxocyclohexyl.

“Alkenyl” includes alkyl moieties having at least one carbon-carbon double bond wherein alkyl is as defined herein; e.g. ethenyl and propenyl.

“Acyl” is —C(═O)—R^{B} wherein R^{B} is hydrogen, C₁₋₆alkyl, C₃₋₇cycloalkyl, C₅₋₁₀aryl and the like; e.g. formyl, acetyl, propionyl, benzoyl and the like.

“Amino” is —NR^{C}R^{D} wherein R^{C} and R^{D} are each independently hydrogen or (C₁-C₆)alkyl.

“Amido” includes the groups —C(═O)—NR^{E}R^{F} (C-amido) and —NR^{E}—C(═O)R^{F} (N-amido), wherein R^{E} and R^{F} are each independently hydrogen, C₁₋₆alkyl or C₁₋₆alkoxy.

“Aryl” includes an organic radical derived from an aromatic hydrocarbon by removal of one hydrogen, such as phenyl, naphthyl, indenyl, indanyl, and fluorenyl; and fused ring groups wherein at least one ring is aromatic.

“Oxo” is ═O.

“Heterocyclic” refers to non-aromatic cyclic groups containing one or more heteroatoms, preferably from one to four heteroatoms, each selected from O, S and N. Heterocyclic groups also include ring systems substituted with one or more oxo moieties. Examples of heterocyclic groups are aziridinyl, azetidinyl, pyrrolidinyl, piperidinyl, azepinyl, piperazinyl, 1,2,3,6-tetrahydropyridinyl, oxiranyl, oxetanyl, tetrahydrofuranyl, tetrahydrothienyl, tetrahydropyranyl, tetrahydrothiopyranyl, morpholino, thiomorpholino, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, quinolizinyl, quinuclidinyl, 1,4-dioxaspiro[4.5]decyl, 1,4-dioxaspiro[4.4]nonyl, 1,4-dioxaspiro[4.3]octyl, and 1,4-dioxaspiro[4.2]heptyl.

“Heteroaryl” refers to aromatic groups containing one or more heteroatoms (O, S, or N), preferably from one to four heteroatoms. A multicyclic group containing one or more heteroatoms wherein at least one ring of the group is aromatic is a “heteroaryl” group. The heteroaryl groups of this invention can also include ring systems substituted with one or more oxo moieties. Examples of heteroaryl groups are pyridinyl, pyridazinyl, imidazolyl, pyrimidinyl, pyrazolyl, triazolyl, pyrazinyl, quinolyl, isoquinolyl, 1,2,3,4-tetrahydroguinolyl, tetrazolyl, furyl, thienyl, isoxazolyl, thiazolyl, oxazolyl, isothiazolyl, pyrrolyl, indolyl, benzimidazolyl, benzofuranyl, cinnolinyl, indazolyl, indolizinyl, phthalazinyl, triazinyl, 1,2,4-trizainyl, 1,3,5-triazinyl, isoindolyl, 1-oxoisoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

“Heterobicyclic” refers to non-aromatic two-ringed cyclic groups, including bridged ring systems, wherein at least one of the rings contains a heteroatom of O, S or N, including without limitation azabicyclics such as 3-azabicyclo[3.1.0]hexanyl and 3-azabicyclo[4.1.0]heptanyl.

The foregoing groups, as derived from the compounds listed above, may be C-attached or N-attached where such is possible. For instance, a group derived from pyrrole may be pyrrol-1-yl (N-attached) or pyrrol-3-yl (C-attached). The terms referring to the groups also encompass all possible tautomers.

“Treatment” and “treating” refers to reversing, alleviating, inhibiting the progress of, or preventing the disorder or condition to which such term applies, or one or more symptoms of such condition or disorder. As used herein, the term also encompasses, depending on the condition of the patient, preventing the disorder, including preventing onset of the disorder or of any symptoms associated therewith, as well as reducing the severity of the disorder or any of its symptoms prior to onset. “Treating” as used herein refers also to preventing a recurrence of a disorder. The term “treatment”, as used herein, refers to the act of treating, as “treating” is defined immediately above.

“Mammal” refers to any member of the class “Mammalia”, including, but not limited to, humans, dogs, and cats.

NK-Mediated Conditions

The invention also pertains to a method of treating a mammal for conditions mediated by neurokinins which comprises administering to a mammal in need of such treatment a therapeutically effective amount of the compound of Formula I.

NK-mediated conditions contemplated for treatment by the present invention include without limitation the following: neurokinin-mediated diseases such as collagenosis, dysfunction of the urinary tract, hemorrhoids, nausea, vomiting, and pain; inflammatory diseases of the respiratory tract, allergic diseases of the respiratory tract, eye diseases, skin diseases, diseases of the gastrointestinal tract, diseases of the joints. Also contemplated is treatment of diseases of the central nervous system (CNS) including without limitation dementia, Alzheimer's disease, schizophrenia, psychosis, depression, headaches, migraine headache or a tension headache and epilepsy; and treatment and/or prevention of CNS disorders such as major depressive disorders including bipolar depression, unipolar depression, single or recurrent major depressive episodes with or without psychotic features, catatonic features, melancholic features, atypical features or postpartum onset, the treatment of anxiety and the treatment of panic disorders. Other mood disorders encompassed within the term major depressive disorders include dysthymic disorder with early or late onset and with or without atypical features, neurotic depression, post traumatic stress disorders and social phobia; dementia of Alzheimer's type, with early or late onset, disorders induced by alcohol, amphetamines, cocaine, hallucinogens, inhalants, opiods, phecyclidine, sedatives, hypnotics, anxiolytics and other substances. The invention is also useful in the treatment of emesis, i.e. nausea, retching and vomiting; including acute emesis, delayed emesis (including chemotheraphy-induced delayed emesis) and anticipatory emesis.

In another practice, the compound of Formula I may be used in conjunction with one or more other therapeutic agents, e.g. different antidepressant agents such as tricyclic antidepressants (e.g. amitriptyline, dothiepin, doxepin, trimipramine, butripyline, clomipramine, desipramine, imipramine, iprindole, lofepramine, nortriptyline or protriptyline), monoamine oxidase inhibitors (e.g. isocarboxazid, phenelzine or tranylcyclopramine) or 5-HT re-uptake inhibitors (e.g. fluvoxamine, sertraline, fluoxetine or paroxetine), and/or with antiparkinsonian agents such as dopaminergic antiparkinsonian agents (e.g. levodopa, preferably in combination with a peripheral decarboxylase inhibitor e.g. benserazide or carbidopa, or with a dopamine agonist e.g., bromocriptine, lysuride or pergolide).

In a preferred practice, the compound of Formula I is used in combination with a 5-HT re-uptake inhibitor (e.g. fluvoxamine, sertraline, fluoxetine or paroxetine), preferably sertraline (or a pharmaceutically acceptable salt or polymorph thereof as would be understood by the artisan) as psychotherapeutics and may be used in the treatment or prevention of disorders the treatment or prevention of which is facilitated by modulating serotonergic neurotransmission such as hypertension, depression (e.g. depression in cancer patients, depression in Parkinson's patients, postmyocardial infarction depression, subsyndromal symptomatic depression, depression in infertile women, pediatric depression, major depression, single episode depression, recurrent depression, child abuse induced depression, and post partum depression), generalized anxiety disorder, phobias (e.g. agoraphobia, social phobia and simple phobias), posttraumatic stress syndrome, avoidant personality disorder, premature ejaculation, eating disorders (e.g. anorexia nervosa and bulimia nervosa), obesity, chemical dependencies (e.g. addictions to alcohol, cocaine, heroin, phenobarbital, nicotine and benzodiazepines), cluster headache, migraine, pain, Alzheimer's disease, obsessive-compulsive disorder, panic disorder, memory disorders (e.g. dementia, amnestic disorders, and age-related cognitive decline (ARCD)), Parkinson's diseases (e.g. dementia in Parkinson's disease, neuroleptic-induced parkinsonism and tardive dyskinesias), endocrine disorders (e.g. hyperprolactinaemia), vasospasm (particularly in the cerebral vasculature), cerebellar ataxia, gastrointestinal tract disorders (involving changes in motility and secretions, negative symptoms of schizophrenia, premenstrual syndrome, fibromyalgia syndrome, stress incontinence, Tourette's syndrome, trichotillomania, kleptomania, male impotence, cancer (e.g. small cell lung carcinoma), chronic paroxysmal hemicrania and headache (associated with vascular disorders).

Sertraline, (1S-cis)-4-(3,4-dichlorophenyl)-1,2,3,4-tetrahydro-N-methyl-1-naphthalenamine, has the chemical formula C₁₇H₁₇NC₁₂; its synthesis is described in U.S. Pat. No. 4,536,518 incorporated herein by reference. Sertraline hydrochloride is useful as an antidepressant and anorectic agent, and is also useful in the treatment of depression, chemical dependencies, anxiety obsessive compulsive disorders, phobias, panic disorder, post traumatic stress disorder, and premature ejaculation.

Activity of the active combination as antidepressants and related pharmacological properties can be determined by methods (1)-(4) below, which are described in Koe, B. et al., Journal of Pharmacology and Experimental Therapeutics, 226 (3), 686-700 (1983). Specifically, activity can be determined by studying (1) their ability to affect the efforts of mice in escaping a swim-tank (Porsolt mouse “behavior despair” test), (2) their ability to potentiate 5-hydroxytryptophan-induced behavioral symptoms in mice in vivo, (3) their ability to antagonize the serotonin-depleting activity of p-chloroamphetamine hydrochloride in rat brain in vivo, and (4) their ability to block the uptake of serotonin, norepinephrine and dopamine by synaptosomal rat brain cells in vitro. The ability of the active combination to counteract reserpine hypothermia in mice in vivo can be determined according to the methods described in U.S. Pat. No. 4,029,731.

Administration

The compound of the invention may be administered either alone or in combination with pharmaceutically acceptable carriers, in either single or multiple doses. Suitable pharmaceutical carriers include inert solid diluents or fillers, sterile aqueous solutions and various organic solvents. The pharmaceutical compositions formed thereby can be readily administered in a variety of dosage forms such as tablets, powders, lozenges, liquid preparations, syrups, injectable solutions and the like. These pharmaceutical compositions can optionally contain additional ingredients such as flavorings, binders, excipients and the like. Thus the compound of the invention may be formulated for oral, buccal, intranasal, parenteral (e.g. intravenous, intramuscular or subcutaneous), transdermal (e.g. patch) or rectal administration or in a form suitable for administration by inhalation or insufflation. E.g. for oral administration, the pharmaceutical compositions may take the form of tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium phosphate); lubricants (e.g. magnesium stearate, talc or silica); disintegrants (e.g. potato starch or sodium starch glycolate); or wetting agents (e.g. sodium lauryl sulphate). The tablets may be coated by methods known in the art. Liquid preparations for oral administration may take the form of e.g. solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, methyl cellulose or hydrogenated edible fats); emulsifying agents (e.g. lecithin or acacia); non-aqueous vehicles (e.g. almond oil, oily esters or ethyl alcohol); and preservatives (e.g. methyl or propyl p-hydroxybenzoates or sorbic acid). For buccal administration, the composition may take the form of tablets or lozenges formulated in conventional manner.

The compound of the invention may be formulated for parenteral administration by injection, including using conventional catheterization techniques or infusion. Formulations for injection may be presented in unit dosage form, e.g. in ampules or in multi-dose containers, with an added preservative. They may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g. sterile pyrogen-free water, before use.

The compound of the invention may also be formulated in rectal compositions such as suppositories or retention enemas, e.g. containing conventional suppository bases such as cocoa butter or other glycerides.

For intranasal administration or administration by inhalation, the compound of the invention is conveniently delivered in the form of a solution or suspension from a pump spray container that is squeezed or pumped by the patient or as an aerosol spray presentation from a pressurized container or a nebulizer, with the use of a suitable propellant, e.g. dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made e.g. from gelatin) for use in an inhaler or insufflator may be formulated containing a powder mix of a compound of the invention and a suitable powder base such as lactose or starch.

A proposed dose of the active compounds of the invention for oral, parenteral or buccal administration to the average adult human for the treatment of the conditions referred to above is about 0.1 to about 200 mg of the active ingredient per unit dose which could be administered, for example, 1 to 4 times per day.

Aerosol formulations for treatment of the conditions referred to above in the average adult human are preferably arranged so that each metered dose or “puff” of aerosol contains about 20 mg to about 1000 mg of the compound of the invention. The overall daily dose with an aerosol will be within the range of about 100 mg to about 10 mg. Administration may be several times daily, e.g. 2, 3, 4 or 8 times, giving for example, 1, 2 or 3 doses each time.

In connection with the use of the compound of the invention with a 5-HT re-uptake inhibitor, preferably sertraline, for the treatment of subjects possessing any of the above conditions, it is to be noted that these may be administered either alone or in combination with pharmaceutically acceptable carriers by either of the routes previously indicated, and that such administration can be carried out in both single and multiple dosages. More particularly, the active combination can be administered in a wide variety of different dosage forms, i.e. they may be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of Formula I are present in such dosage forms at concentration levels ranging from about 0.5% to about 90% by weight of the total composition, i.e., in amounts which are sufficient to provide the desired unit dosage and a 5-HT re-uptake inhibitor, preferably sertraline, is present in such dosage forms at concentration levels ranging from about 0.5% to about 90% by weight of the total composition, i.e. in amounts which are sufficient to provide the desired unit dosage.

A proposed daily dose of the compound of the invention in the combination formulation (a formulation containing the compound of the invention and a 5-HT re-uptake inhibitor) for oral, parenteral, rectal or buccal administration to the average adult human for the treatment of the conditions referred to above is from about 0.01 mg to about 2000 mg, preferably from about 0.1 mg to about 200 mg of the active ingredient of Formula I per unit dose which could be administered, for example, 1 to 4 times per day.

A proposed daily dose of a 5-HT re-uptake inhibitor, preferably sertraline, in the combination formulation for oral, parenteral or buccal administration to the average adult human for the treatment of the conditions referred to above is from about 0.1 mg to about 2000 mg, preferably from about 1 mg to about 200 mg of the 5-HT re-uptake inhibitor per unit dose which could be administered, for example, 1 to 4 times per day.

A preferred dose ratio of sertraline to an active compound of this invention in the combination formulation for oral, parenteral or buccal administration to the average adult human for the treatment of the conditions referred to above is from about 0.00005 to about 20000; preferably from about 0.25 to about 2000.

Aerosol combination formulations for treatment of the conditions referred to above in the average adult human are preferably arranged so that each metered dose or “puff” of aerosol contains from about 0.01 mg to about 100 mg of the active compound of this invention, preferably from about 1 mg to about 10 mg of such compound. Administration may be several times daily, e.g. 2, 3, 4 or 8 times, giving for example, 1, 2 or 3 doses each time.

Aerosol formulations for treatment of the conditions referred to above in the average adult human are preferably arranged so that each metered dose or “puff” of aerosol contains from about 0.01 mg to about 2000 mg of a 5-HT re-uptake inhibitor, preferably sertraline, preferably from about 1 mg to about 200 mg of sertraline. Administration may be several times daily, for example 2, 3, 4 or 8 times, giving for example, 1, 2 or 3 doses each time.

As previously indicated, a 5-HT re-uptake inhibitor, preferably sertraline, in combination with compounds of Formula I are readily adapted to therapeutic use as antidepressant agents. In general, these antidepressant compositions containing a 5-HT re-uptake inhibitor, preferably sertraline, and a compound of Formula I are normally administered in dosages ranging from about 0.01 mg to about 100 mg per kg of body weight per day of a 5-HT re-uptake inhibitor, preferably sertraline, preferably from about 0.1 mg to about 10 mg per kg of body weight per day of sertraline; with from about 0.001 mg. to about 100 mg per kg of body weight per day of a compound of Formula I, preferably from about 0.01 mg to about 10 mg per kg of body weight per day of a compound of Formula I, although variations will necessarily occur depending upon the conditions of the subject being treated and the particular route of administration chosen.

The following Schemes and Examples are offered in illustration of the present invention; they are not to constrain the scope of same in any way.

General Synthetic Schemes

The following schemes are representative of methods useful in synthesizing the compound of the present invention.

The compounds of Formula I wherein a piperidine core is central and a=1 may be conveniently prepared as disclosed in Scheme 1.

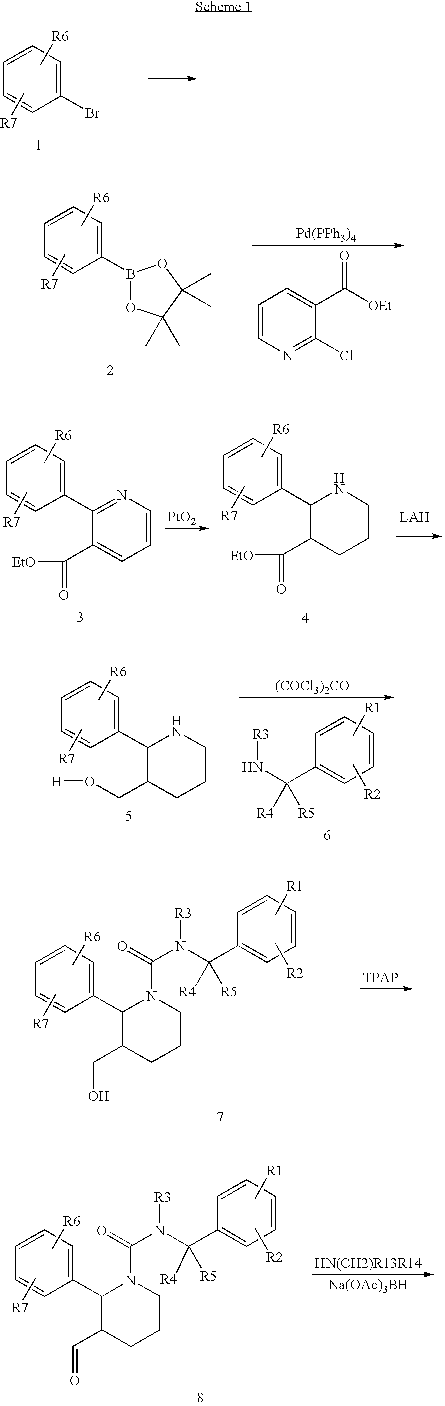

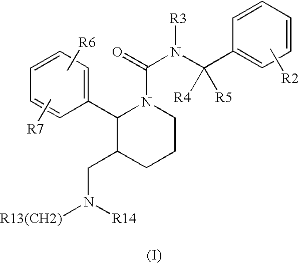

In Scheme 1, an appropriately-substituted aryl/heteroaryl bromide (1) is reacted with 4,4,5,5,-tetramethyl-[1,3,2]dioxaborolane, in the presence of a palladium (Pd) catalyst, preferably PdCl₂(dppf), and in the presence of triethyl amine, in a reaction inert solvent, such as dioxane, at a reaction temperature of between about room temperature (rt) and about the reflux temperature of the solvent employed gives boronic ester (2). Reaction of boronic ester (2) with ethyl-2-chloro-nicotinate, in the presence of a Pd catalyst, preferably Pd(Ph₃)₄, in dichloroethane, at the reflux temperature, gives pyridine (3). Reduction of pyridine (3) using known methods gives (4); For example reaction of pyridine (3) in ethanol, in the presence of trifluoroacetic acid (TFA), PtO₂, and H₂ (50 psi) gives piperidine (4). Reduction of ester (4) with lithium aluminum hydride (LAH), in a reaction of inert solvent, preferably tetrahydrofuran (THF), at a reaction temperature of about around 0° C. to around rt gives alcohol (5). Piperidine (5) can converted to the urea (7) according to known methods. The coupling of an amine such as (6) with piperidine (5) is typically performed in a reaction-inert solvent such as methylene chloride or dichloromethane, at a temperature of between about −78° C. to the reflux temperature of the solvent employed, preferable at about 0° C. to about 55° C., in the presence of a carbonyl equivalent, selected from phosgene, triphosgene, or carbonyldiimidazole, and in the presence of a trialkylamine base, such a triethylamine, diisopropylethylamine to afford (7). Reaction of alcohol (7) with an oxidizing reaction, preferably tetra-n-propyl ammonium perruthenate (TPAP), in the presence of 4-methyl morpholine N-oxide in a reaction inert solvent, for example dichloromethane gives aldehyde (8). Reductive amination of (8), with an amine, primary or secondary, in a reaction of inert solvent, preferable toluene, tetrahydrofuran, methanol or dichloromethane, in the presence of a reducing reagent, such as NaBH₄, and Na(OAc)₃BH gives a compound of Formula I.

The compounds of Formula I wherein a piperidine core is central and a=0 may be conveniently prepared as disclosed in Scheme 2.

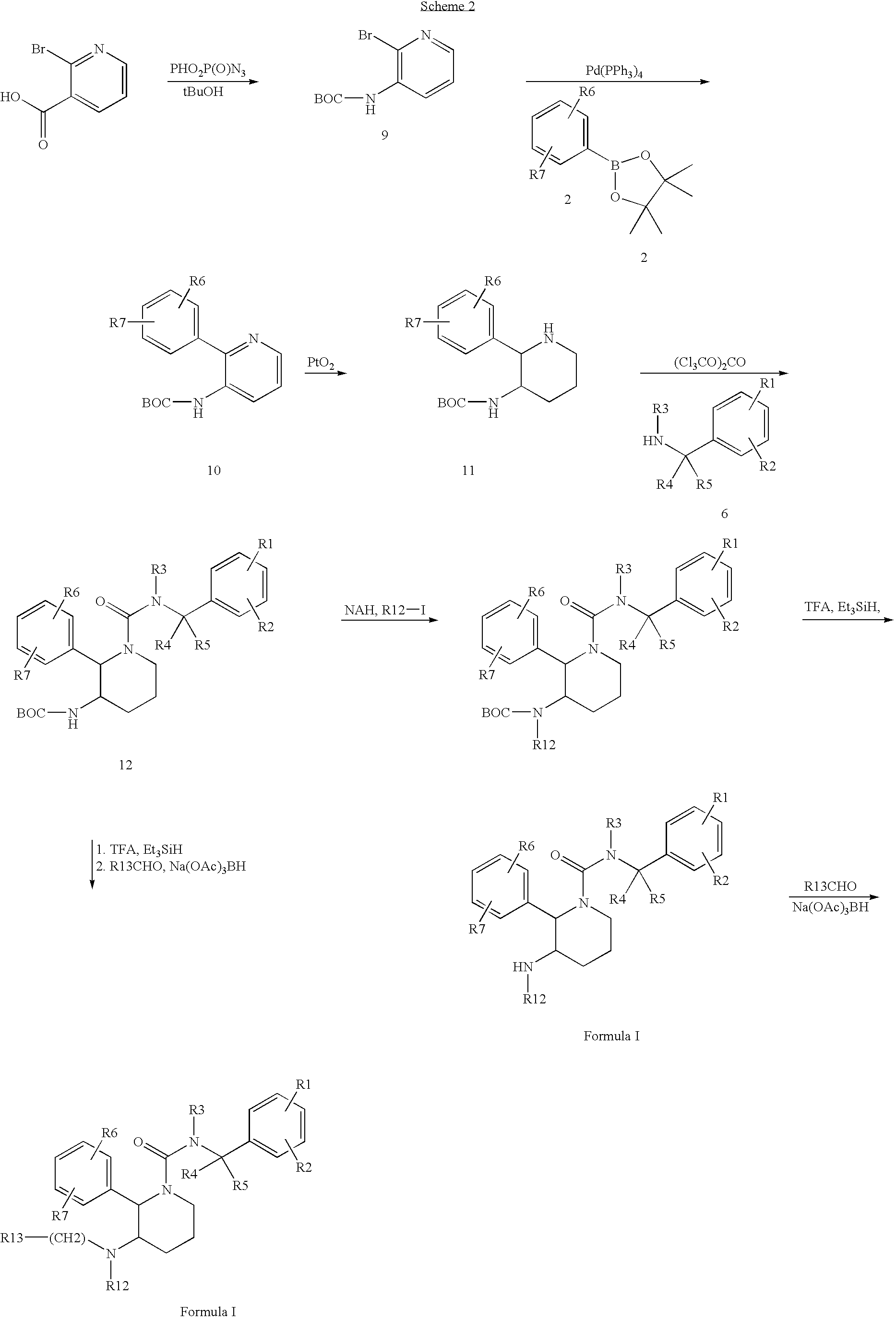

In Scheme 2, 2-bromo-nicotinic acid can be converted to the N-BOC pyridine (9) using known methods. For example reaction of 2-bromonicotinic acid with diphenylphosphoryl azide, in the presence of t-butyl alcohol gives N-BOC pyridine (9). Reaction of bromide (2), as described in Scheme I with boronic ester (2) gives (3). Reduction of the pyridine ring using conditions outlined in Scheme I, gives piperidine (11). The piperidine intermediate (11) can be converted to the urea intermediate (12) according to known methods. The coupling of an amine such as (6) with intermediate (11) is typically performed in a reaction-inert solvent such as methylene chloride or dichloromethane, at a temperature of between about −78° C. to the reflux temperature of the solvent employed, preferably at about 0° C. to rt, in the presence of a carbonyl equivalent, selected from phosgene, triphosgene, or carbonyldiimidazole, and in the presence of a trialkylamine base, such a triethylamine, diisopropylethylamine to afford (12). An intermediate of the general structure (12) can be converted to Formula I by first removal of the protecting group by known methods. Preferably, (12) is treated with trifluoroacetic acid, in the presence of triethylsilane. Reductive amination of the resulting amine with an appropriate aldehyde, in the presences of a reducing reagent such as sodium triacetoxyborohydride affords a compound of Formula I. Alternatively, intermediate (12) can be alkylated by known methods; for example, treatment of (12) with NaH, in a reaction inert solvent, such as THF, and in the presence of an alkyl halide, gives (13). Removal the protecting group of (13) can be accomplished by reaction of (13), in a reaction inert solvent, such a methylene chloride, in the presence of triflouroacetic acid, and triethylsilane to give a compound of Formula I. Further, reductive amination of the resulting secondary amine with an appropriate aldehyde or ketone, and sodium triacetoxyborohydride affords a compound of Formula I.

The compounds of Formula I wherein a pyrrolidine core is central and a=1 maybe conveniently prepared as disclosed in Scheme 3.

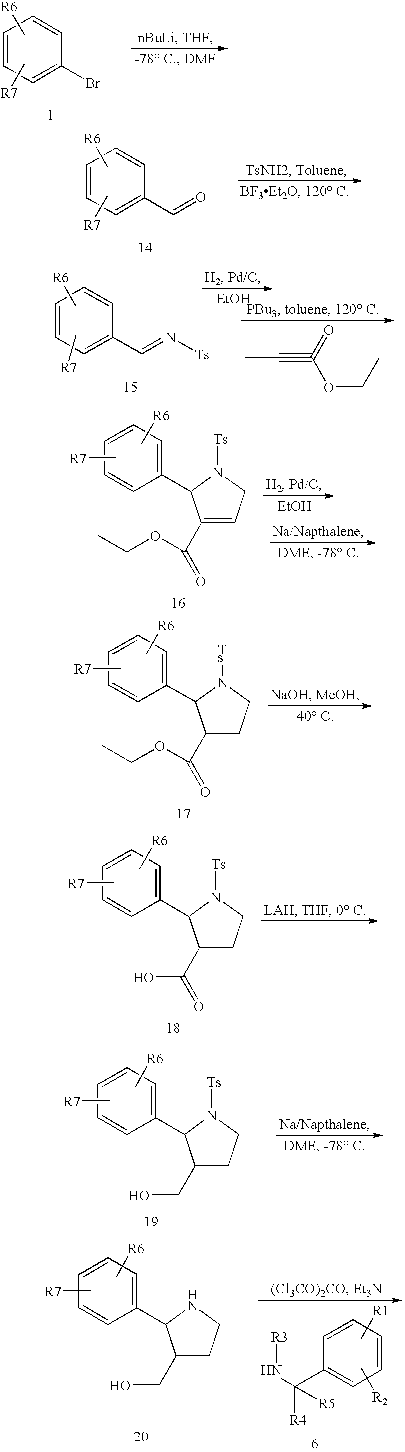

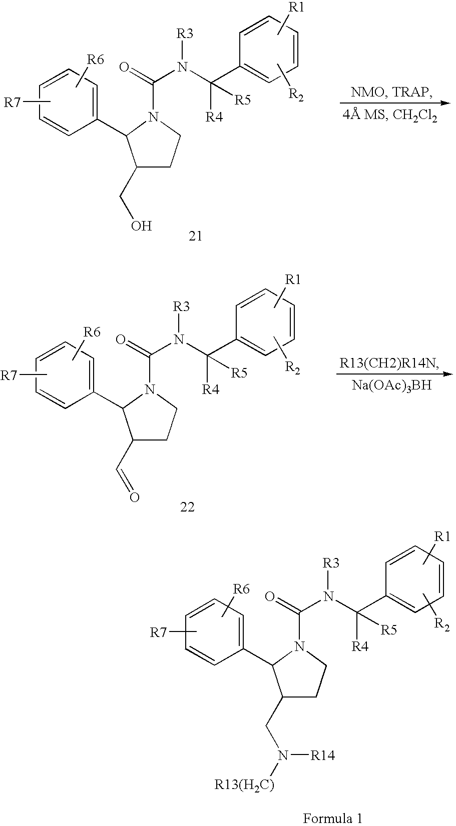

In Scheme 3, an appropriately substituted aryl/heteroaryl bromide (1) is reacted with an organo metallic reagent, such as organo lithium, organo magnesium halide, at a reaction temperature of between about −78° C. and about rt for an appropriate reaction time, e.g. at around 15 min, and then dimethyl formamide is added to give aldehyde (14). Reaction of aldehyde (14) in a reaction of inert solvent, such as toluene, at the reflux temperature of the solvent employed, in the presence of p-toluene sulfonamide and in the presence of a Lewis acid, preferably boron trifluoride etherate gives imine (15). Reaction of imine (15) with 2-butynoic acid ethyl ester, in the presence of tributyl phosphine, in a reaction of inert solvent, such at toluene, at the reflux temperature gives (16). Reduction of the olefin can be accomplished under standard hydrogenation conditions that appear in the literature. The preferred method of reduction is by reaction of (16) in the presence of palladium on carbon, and in the presence of hydrogen, at about a pressure of 45 psi, in a lower alcohol solvent, such as methanol, ethanol to give (17). Hydrolysis and isomerization of the ester (17) is accomplished in a lower alcohol solvent, such as methanol, ethanol, in the presence of a base, where preferred bases are potassium hydroxide, and sodium hydroxide, at a reaction temperature from about 0° C. to the reflux temperature of the solvent employed, gives (18). Acid (18) is reacted with a reducing reagent, such as lithium aluminum hydride, in a reaction of inert solvent, where THF is preferred at a reaction temperature from about 0° C. to rt, gives (19). Removal of the tosyl protecting group can be accomplished using known methods. A preferred method is by reaction of (19) with sodium/naphthalene in a reaction inert solvent, where dimethoxy ethane is the preferred solvent, at a reaction temperature of about −78° C. to rt, where about around −78° C. is preferred to give (20). The coupling of an amine (6) with intermediate (20) is typically performed in a reaction-inert solvent such as methylene chloride or dichloromethane, at a temperature between about −78° C. to the reflux temperature of the solvent employed, preferably at about 0° C. to the reflux temperature of the solvent employed, where the preferred reaction temperature is around about 55° C., in the presence of a carbonyl equivalent, selected from phosgene, triphosgene, or carbonyldiimidazole, and in the presence of a trialkylamine base, such a triethylamine, diisopropylethylamine, to afford (21). The oxidation of alcohol (21) can be accomplished using well-known conditions that appear in the literature. The preferred method is by reaction of (21) in a reaction of inert solvent, preferable methylene chloride, in the presence of molecular sieves, N-methylmorpholine N-oxide, and tetrapropylammonium perruthenate, at about 0° C. to rt, gives (22). Reductive amination of (22) is preferably accomplished by reaction of (22) in a reaction inert solvent, such as methylene chloride, dichloroethane, tetrahydrofuran, where preferred solvent is tetrahydrofuran, in the presence of an appropriate amine, primary or secondary amine, and in the presence of Na(OAc)₃BH to give a compound of Formula I.

The compounds of Formula I wherein a pyrrolidine core is central and a=0 maybe conveniently prepared as disclosed in Scheme 4.

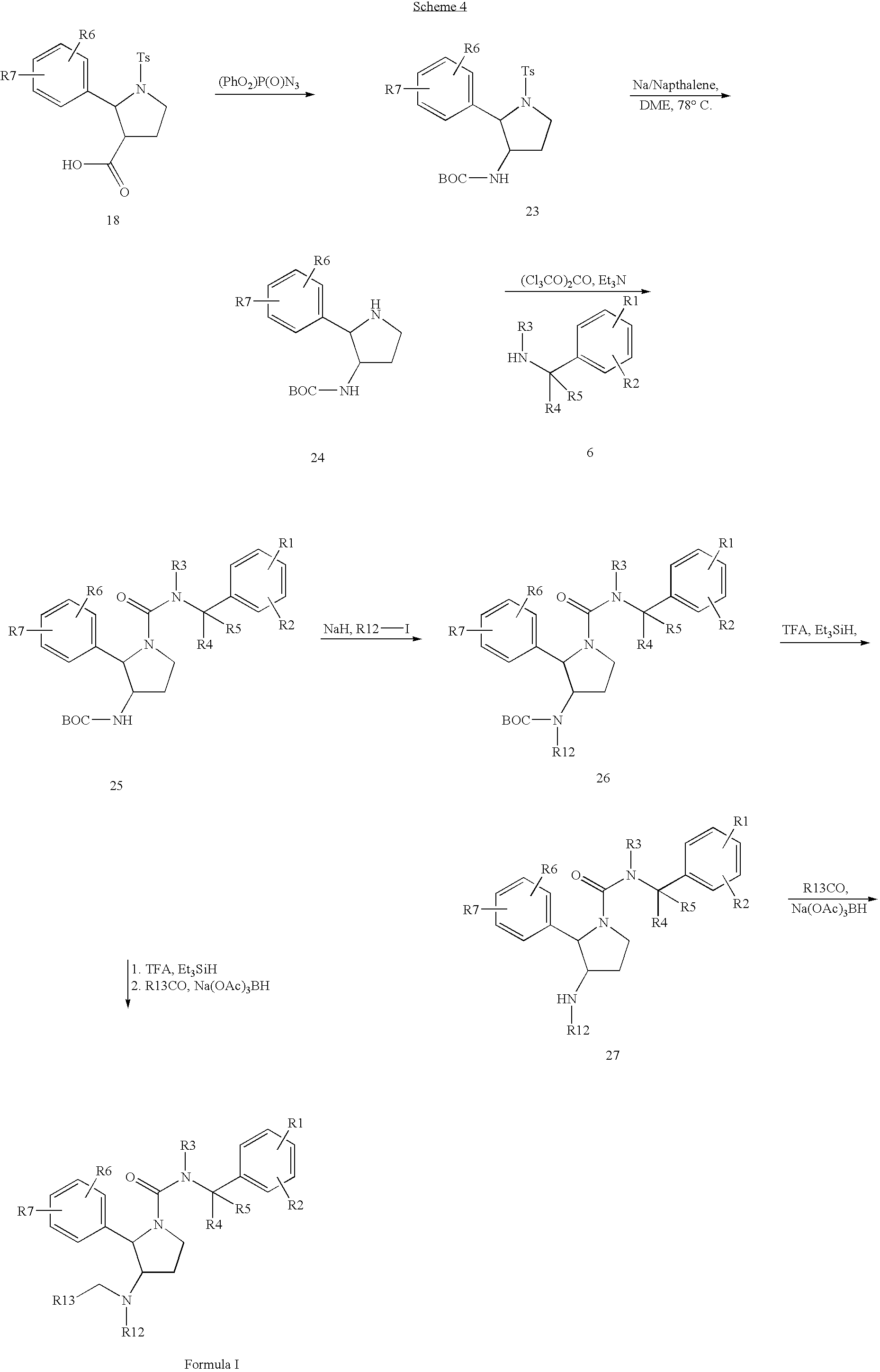

In Scheme 4, acid (18) from Scheme 3 is converted to the BOC protected amine (23) as described in Scheme 2. Removal of the tosyl protecting group can be accomplished as described in Sceme 3. Coupling of an amine (6) with intermediate (24) is typically performed as described in Scheme 3 to give urea (25). Conversion of BOC-amine (25) to compounds of Formula I can be accomplished using conditions described in Scheme 2.

The activity of the compounds of the present invention as substance P antagonists is determined by their ability to inhibit the binding of substance P at its receptor sites in IM-9 cells employing radioactive ligands. The substance P antagonist activity of the compounds described herein is evaluated by using the standard assay procedure described by D. G. Payan et al., as reported in the The Journal of Immunology, 133, 3260 (1984). This method essentially involves determining the concentration of the individual compound required to reduce by 50% the amount of radiolabelled substance P ligands at their receptor sites in said isolated cow tissues or IM-9 cells, thereby affording characteristic IC₅₀ values for each compound tested. More specifically, inhibition of [³H]SP binding to human IM-9 cells by compounds are determined in assay buffer (50 mM Tris-HCl (pH 7.4), 1 mM MnCl₂, 0.02% bovine serum albumin, bacitracin (40 μg/ml), leupeptin (4 μg/ml), chymostatin (2 μg/ml) and phosphoramidon (30 μg/ml)). The reaction is initiated by the addition of cells to assay buffer containing 0.56 nM [³H]SP and various concentrations of compounds (total volume 0.5 ml) and allowed to incubate for 120 min at 4° C. Incubation is terminated by filtration onto GF/B filters (presoaked in 0.1% polyethylenamine for 2 hours). Nonspecific binding is defined as the radioactivity remaining in the presence of 1 μM SP. The filters are placed into tubes and counted using liquid scintillation counter.

The activity of the compounds of this invention against generalized anxiety disorder can be determined by inhibition of GR73632-induced tapping test in gerbils. More specifically, gerbils are lightly anesthetized with ether and the skull surface is exposed. GR73632 or vehicle (PBS, 5 μl) are administered directly into the lateral ventricles via a 25 gauge needle inserted 4.5 mm below bregma (preceded by pretreatment with an antagonist, 0.1-32.0 mg/kg, s.c. or p.o.). Following injection, gerbils are placed in 1 L beaker individually and monitored for repetitive hind paw tapping. Some compounds prepared in the following Examples were tested in accordance with these testing methods. As a result, it was found that the compounds of the present inventions have good antagonist activity toward substance P, particularly good activity against CNS disorders with decreased side effects.

The following examples are illustrative only; they are not restrictive.

### EXAMPLES

Intermediates

Intermediate 1:

### 2-(4-Fluoro-2-methyl-phenyl)-4,4,5,5-tetramethyl-[1,3,2]dioxaborolane

PdCl₂(dppf)-CH₂Cl₂ (2.9 g, 4.0 mmol) was flushed with N₂. Dioxane (200 mL) was separately flushed with N₂ and then added into the reaction flask containing the catalyst. 2-Bromo-5-fluorotoluene (25.0 g, 132.3 mmol) was then added, followed by Et₃N (55.3 mL, 397 mmol). 4,4,5,5-Tetramethyl-[1,3,2]dioxaborolane (28.8 mL, 198 mmol) was added to the reaction mixture portionwise over 20 min at rt. Some gas evolution and exotherm may be noted. Once the addition was complete, the reaction was heated in an oil bath at 80° C. under N₂ and monitored by GC-MS. As the reaction was heated, fresh catalyst was added (5.0 g, 6.8 mmol) every 8-12 hours until starting material was consumed (3.0 equivalents of catalyst total) over 96 hours. The reaction was then cooled to rt and quenched with aqueous NH₄Cl (25 mL) and filtered through a pad of celite. The crude filtrate was concentrated and purified by flash chromatography on a 75S Biotage silica gel column eluting with 10% EtOAc/Hexanes, collecting 250 mL factions. The product containing fractions (1-2) were collected and concentrated under reduced pressure to give a colorless crystalline solid (18.6 g, 60% yield); Rf 0.75 (10% EtOAc/Hexanes); GC-MS: HP-1 (12 m×0.200 mm×0.33 um) column, 65-300° C. with a 30° C./min time ramp over 9.0 min, retention time=3.56, 236 [M]; 400 MHz ¹HNMR (CDCl₃) δ 7.75 (t, J=7.5 Hz, 1H); 6.87-6.83, (m, 2H); 2.54 (s, 3H); 1.34 (s, 12H). 100 MHz ¹³C NMR (CDCl₃) δ 166.1, 163.6, 148.4, 138.4, 138.3, 116.9, 116.7, 112.0, 111.9, 83.7, 25.1, 22.4.

Intermediate 2:

### 4-Fluoro-2-methyl-benzaldehyde

2-Bromo-5-fluorotoluene (15.0 g, 79.3 mmol) was dissolved in anhydrous THF and cooled to −78° C. in an acetone/dry ice bath. N-butyl-lithium (48.0 mL, 119 mmol) was added dropwise down the side of the reaction flask and the resulting solution was stirred for 10 min. 64.0 mL of anhydrous DMF (793 mmol) was then added in the same fashion. After 1 hour, the reaction was quenched with cold aqueous NH₄Cl and diluted with 500 mL Et₂O and washed with water (3×300 mL). The organic layer was dried over MgSO₄, filtered and concentrated under reduced pressure. The crude material was then equipped with a distillation apparatus and the oil bath was heated to 110° C. Product containing fractions were collected to give a colorless oil, (10.0 g , 72.4 mmol, 92% yield); LRMS m/z (APCI+) 139 [M+H]; 400 MHz ¹HNMR (CDCl₃) δ 10.91 (s, 1H); 7.81 (dd, J=6.2, 2.5 Hz, 1H); 7.03 (ddd, J=8.2, 8.2, 2.5 Hz, 1H); 6.95 (ddd, J=9.5, 2.5, 0.0 Hz, 1H); 2.67 (s, 3H).

Intermediate 3:

### N-(4-Fluoro-2-methyl-benzylidene)-4-methyl-benzenesulfonamide

Intermediate 2 (42.5 g, 308 mmol) was dissolved in 400 mL of anhydrous toluene. p-Toluenesulfonamide (47.4 g, 277 mmol) was added, followed by the dropwise addition of BF₃.OEt₂ (0.6 mL, 6.2 mmol). The reaction was then heated to reflux in an oil bath at 120° C., equipped with a Dean Stark trap and monitored by GC-MS. After 12 hours, another addition of BF₃.OEt₂ was added and the reaction heated for an additional 12 hours. The reaction was cooled to rt and quenched with 250 mL of aqueous saturated NaHCO₃. The suspension was then extracted with EtOAc (2×400 mL) and the combined organics were dried over Na₂SO₄, filtered and concentrated under reduced pressure. The crude material was crystallized with hot Et₂O to give clean product as colorless needles (68.4 g, 234.7 mmol, 76% yield); Rf 0.8 (50% EtOAc/Hexanes); LRMS m/z (APCI+) 292 [M+H]; GC-MS: HP-1 (12 m×0.200 mm×0.33 um) column, 105-300° C. with an 18° C./min time ramp over 12.0 min, retention time=8.34, 291 [M]; 400 MHz ¹HNMR (CDCl₃) δ 9.27 (s, 1H); 8.04 (ddd, J=5.8FH, 3.7, 3.7 Hz, 1H); 7.88 (d, J=8.3 Hz, 2H); 7.34 (d, J=7.9 Hz, 2H); 6.99-6.95 (m, 2H); 2.61 (s, 3H); 2.44 (s, 3H); 100 MHz ¹³C NMR (CDCl₃) δ 167.9, 167.4, 145.9, 144.8, 135.5, 133.7, 133.6, 130.1, 128.2, 127.3, 118.7, 118.5, 114.6, 114.4, 21.9, 20.0.

Intermediate 4:

### 2-(4-Fluoro-2-methyl-phenyl)-1-(toluene-4-sulfonyl)-2,5-dihydro-1H-pyrrole-3-carboxylic acid ethyl ester

Intermediate 3 (74.5 g, 256 mmol) and ethyl-2-butynoate (29.8 mL, 256 mmol) were dissolved in 300 mL of anhydrous toluene. Tributylphosphine (6.5 mL, 25.6 mmol) was then added and the reaction was heated to reflux in an oil bath at 120° C. for 2 h. The reaction was cooled to rt and concentrated under reduced pressure. Purification was accomplished by flash chromatography on a 75L Biotage silica gel column eluting with a gradient of 10%, 20%, 30%, 50% EtOAc/Hexanes, collecting 100 mL fractions. The product containing fractions were collected and concentrated under reduced pressure to give a pale yellow oil. The product was then crystallized from hot isopropyl ether to give a colorless solid (66.9 g, 166 mmol, 65% yield); Rf 0.75 (EtOAc/Hexanes); LRMS m/z (APCI+) 404 [M+H]; 400 MHz ¹HNMR (CDCl₃) δ 7.34 (ddd, J=3.7, 2.1, 2.1 Hz, 2H); 7.13 (d, J=7.9 Hz, 2H); 6.82-6.76 (m, 3H); 6.61 (ddd, J=8.3, 8.3, 2.4 Hz, 1H); 6.00 (ddd, J=4.6, 2.5, 2.5 Hz, 1H); 4.55 (ddd, J=5.0, 2.5, 2.5 Hz, 1H); 4.37 (ddd, J=17.0, 6.2, 2.1 Hz, 1H); 4.06-3.95 (m, 2H); 2.54 (s, 3H); 2.36 (s, 3H); 1.10 (t, J=7.3 Hz, 3H); 100 MHz ¹³C NMR (CDCl₃) δ 163.4, 161.9, 160.9, 143.6, 139.5, 139.4, 136.4, 136.0, 135.5, 133.8, 129.6, 129.5, 129.4, 127.1, 117.1, 116.9, 113.2, 113.0, 64.4, 61.1, 55.1, 21.7, 19.5, 14.1.

Enantiomers Separated:

Intermediate 5:

### 2-(R)-(4-Fluoro-2-methyl-phenyl)-1-(toluene-4-sulfonyl)-2,5-dihydro-1H-pyrrole-3-carboxylic acid ethyl ester

Colorless crystalline solid: Chiralpak AS (10 cm×50 cm) 275 mL/min, 55/45 Heptaine/IPA retention time 7.26 min; [α]₂₂^{D}=+213.63° (c 1.21, CH₂Cl₂); Anal. Calcd for C₂₁H₂₂FNO₄S: C, 62.51; H, 5.50; N, 3.47. Found: C, 62.88; H, 5.42; N, 3.49.

Intermediate 6:

### 2-(S)-(4-Fluoro-2-methyl-phenyl)-1-(toluene-4-sulfonyl)-2,5-dihydro-1H-pyrrole-3-carboxylic acid ethyl ester

pale yellow, crystalline solid: Chiralpak AS (10 cm×50 cm) 275 mL/min, 55/45 Heptaine/IPA retention time 11.42 min; [α]₂₂^{D}=−210.18° (c 1.00, CH₂Cl₂); Anal. Calcd for C₂₁H₂₂FNO₄S: C, 62.51; H, 5.50; N, 3.47. Found: C, 62.50; H, 5.22; N, 3.44.

Intermediate 7:

### Trans-RS,RS-2-(4-Fluoro-2-methyl-phenyl)-1-(toluene-4-sulfonyl)-pyrrolidine-3-carboxylic acid

Intermediate 4 (42.2 g, 0.10 mol) was suspended in 650 mL of EtOH and flushed with N₂. Pd/C (2.10 g, 5% weight) was added and the reaction was flushed with H₂ and then maintained under a H₂ atmosphere at 45 psi for 2 h. 200 mL of EtOAc was then added to completely dissolve the product coming out of solution and the reaction was resubjected to 45 psi H₂ for an additional ½ h. The reaction was then filtered through a plug of celite and concentrated under reduced pressure to give a colorless oil (42.4 g); Rf 0.4 (50% EtOAc/Hexanes).

The crude ester was used directly in the next step. To a solution of 200 mL of MeOH/200 mL of H₂O was added the ester, followed by NaOH pellets (41.6 g, 104 mol). After 15 min. of stirring at rt, the reaction was heated in an oil bath at 65° C. for 1 h. The MeOH was removed under reduced pressure and the remaining aqueous layer was acidified with conc. HCl (pH=3.0) and extracted with CH₂Cl₂ (3×200 mL). Combined organics were dried over Na₂SO₄, filtered and concentrated under reduced pressure to give a colorless solid (38.0 g, 0.10 mol, 97% yield); Rf 0.5 (10% MeOH/CH₂Cl₂); LRMS m/z (APCI+) 376/378 [M−/+H]; 400 MHz ¹HNMR (CDCl₃) δ 7.63 (d, J=8.3 Hz, 2H); 7.29 (dd, J=8.3, 5.8 Hz, 1H); 7.21 (d, J=7.9 Hz, 2H); 6.87-6.80 (m, 2H); 5.25 (d, J=2.9 Hz, 1H); 3.74 (ddd, J=10.8, 7.1, 3.3 Hz, 1H); 3.47 (ddd, J=9.1, 9.1, 7.1 Hz, 1H); 2.77 (dddd, J=3.7, 3.7, 3.7, 3.7 Hz, 1H); 2.38 (s, 3H); 2.33 (s, 3H); 2.20-2.08 (m, 2H); 100 MHz ¹³C NMR (CDCl₃) δ 178.5, 162.1 (d, J_{C-F}=245 Hz), 143.8, 137.0, 136.9, 135.8, 134.7, 129.6, 128.3, 128.2, 127.8, 117.5 (d, J_{C-F}=20 Hz), 113.1 (d, J_{C-F}=20 Hz), 62.2, 51.8, 48.2, 26.9, 21.7, 19.7.

Intermediate 8:

### Trans-RS,RS-[2-(4-Fluoro-2-methyl-phenyl)-1-(toluene-4-sulfonyl)-pyrrolidin-3-yl]-methanol

Intermediate 7 (20.2 g, 53.5 mmol) was dissolved in 200 mL of anhydrous THF under N₂ and cooled to 0° C. in an ice/water bath. A 1M solution of LAH in THF (68.0 mL, 68.0 mmol) was added portionwise over 20 min. Once the addition was complete, the reaction was allowed to warm to rt and stir for 1 h. The reaction was quenched with 2.4 mL H₂O, followed by 2.4 mL of 15% aqueous NaOH and another 7.2 mL of H₂O. The resulting solution was then further dissolved with 150 mL of EtOAc and filtered through a plug of celite. The filtrate was dried over MgSO₄, filtered and concentrated under reduced pressure to give a colorless solid (16.2 g, 44.6 mmol, 83% yield); Rf 0.85 (10% MeOH/CH₂Cl₂); LRMS m/z (APCI+) 364 [M+H]; 400 MHz ¹HNMR (CDCl₃) δ 7.63 (d, J=8.3 Hz, 2H); 7.28 (d, J=7.5 Hz, 2H); 7.21 (dd, J=8.7, 6.2 Hz, 1H); 6.82-6.76 (m, 2H); 4.84 (d, J=3.3 Hz, 1H); 3.65 (ddd, J=12.0, 7.9, 4.2 Hz, 1H); 3.44 (ddd, J=16.2, 8.3, 0.0 Hz, 1H); 3.22 (dd, J=10.8, 6.6 Hz, 1H), 3.13 (dd, J=10.8, 6.6 Hz, 1H); 2.41 (s, 3H); 2.36 (s, 3H); 2.11 (dddd, J=6.6, 6.6, 3.3, 3.3 Hz, 1H); 2.04-1.95 (m, 1H); 1.67-1.60 (m, 2H); 100 MHz ¹³C NMR (CDCl₃) δ 161.8 (d, J_{C-F}=244 Hz), 143.8, 137.0, 136.9, 136.7, 135.3, 129.8, 128.2, 128.2, 127.6, 117.2 (d, J_{C-F}=21 Hz), 112.8 (d, J_{C-F}=21 Hz), 62.7, 62.0, 50.2, 47.9, 25.9, 21.8, 19.7.

Enantiomers Separated:

Intermediate 9:

### [2-(R)-(4-Fluoro-2-methyl-phenyl)-1-(toluene-4-sulfonyl)-pyrrolidin-3-(R)-yl]-methanol

HRMS m/z calculated (Calcd) for C₁₉H₂₃NO₃FS, 364.1382, found, 364.1383; [α]₂₂^{D}=+122.9° (c 1.05, CH₂Cl₂); Anal. Calcd for C₁₉H₂₂FNO₃: C, 62.79; H, 6.10; N, 3.85. Found: C, 62.48; H, 6.00; N, 3.77. 400 MHz ¹HNMR (CDCl₃) δ 7.66 (d, J=8.3 Hz, 2H); 7.29 (d, J=7.9 Hz, 2H); 7.24 (dd, J=8.3, 6.2 Hz, 1H); 6.84-6.79 (m, 2H); 4.87 (d, J=3.1 Hz, 1H); 3.68 (ddd, J=11.9, 8.3, 4.2 Hz, 1H); 3.47 (ddd, J=17.1, 8.3, 0.0 Hz, 1H); 3.24 (dd, J=10.9, 6.7 Hz, 1H), 3.15 (dd, J=10.9 Hz, 6.7 Hz, 1H), 2.44 (s, 3H); 2.38 (s, 3H); 2.13 (dddd, J=18.7, 18.7, 8.8, 8.8 Hz, 1H); 2.06-1.99 (m, 1H); 1.69-1.63 (m, 1H); 1.57 (bs, 1H); 100 MHz ¹³C NMR (CDCl₃) δ 162.9, 160.9, 143.8, 137.0, 136.9, 136.7, 136.7, 135.4, 129.8, 128.3, 128.2, 127.6, 117.3, 117.1, 113.0, 112.8, 62.8, 62.0, 50.2, 48.0, 26.0, 21.8, 19.7.

Intermediate 10:

### 2-(S)-(4-Fluoro-2-methyl-phenyl)-1-(toluene-4-sulfonyl)-pyrrolidin-3-(S)-yl]-methanol

HRMS m/z Calcd for C₁₉H₂₃NO₃FS: 364.1382. found: 364.1383. [α]₂₂^{D}=−116° (c 1.05, CH₂Cl₂); Anal. Calcd for C₁₉H₂₂FNO₃: C, 62.79; H, 6.10; N, 3.85. Found: C, 63.00; H, 6.21; N, 3.73. 400 MHz ¹HNMR (CDCl₃) δ 7.66 (d, J=8.3 Hz, 2H); 7.29 (d, J=7.9 Hz, 2H); 7.24 (dd, J=8.3, 6.2 Hz, 1H); 6.84-6.79 (m, 2H); 4.87 (d, J=3.1 Hz, 1H); 3.68 (ddd, J=11.9, 8.3, 4.2 Hz, 1H); 3.47 (ddd, J=17.1, 8.3, 0.0 Hz, 1H); 3.24 (dd, J=10.9, 6.7 Hz, 1H), 3.15 (dd, J=10.9 Hz, 6.7 Hz, 1H), 2.44 (s, 3H); 2.38 (s, 3H); 2.13 (dddd, J=18.7, 18.7, 8.8, 8.8 Hz, 1H); 2.06-1.99 (m, 1H); 1.69-1.63 (m, 1H); 1.57 (bs, 1H); 100 MHz ¹³C NMR (CDCl₃) δ 162.9, 160.9, 143.8, 137.0, 136.9, 136.7, 136.7, 135.4, 129.8, 128.3, 128.2, 127.6, 117.3, 117.1, 113.0, 112.8, 62.8, 62.0, 50.2, 48.0, 26.0, 21.8, 19.7.

Intermediate 11:

### Trans-RS,RS-[2-(4-Fluoro-2-methyl-phenyl)-pyrrolidin-3-yl]-methanol

A 1M solution of Na/Napthalene was made by dissolving 26.7 g of napthalene and 3.5 g Na in 150 mL of anhydrous DME and stirring this suspension at rt overnight. Intermediate 8 (16.2 g, 44.6 mmol) was separately dissolved in 200 mL of anhydrous DME and cooled to −78° C. in and acetone/dry ice bath. The 1M solution of sodium napthilide (134 mL) was then added dropwise until the dark blue color of the reaction solution remained. The reaction was stirred for an additional 10 min, and then quenched with 50 mL H₂O and warmed to rt. The DME was concentrated off under reduced pressure and the residual oil was dissolved in 1M HCl (200 mL) and extracted with CH₂Cl₂ (2×200 mL). The aqueous layer was then basified with 100 mL 15% NaOH and extracted with fresh CH₂Cl₂ (2×100 mL). The product containing organics were combined, dried over Na₂SO₄, filtered and concentrated under reduced pressure to give a clear, colorless oil (8.8 g, 42.1 mmol, 95% yield); Rf 0.2 (10% MeOH/CH₂Cl₂); LRMS m/z (APCI+) 210 [M+H]; 400 MHz ¹HNMR (CDCl₃) δ 7.33 (dd, J=8.3, 5.8 Hz, 1H); 6.89-6.81 (m, 2H); 4.13 (d, J=7.1 Hz, 1H); 3.61 (dddd, J=10.4, 10.4, 10.4, 5.8 Hz, 2H); 3.22 (ddd, J=10.3, 8.3, 5.0 Hz, 1H); 3.06 (ddd, J=10.4, 8.7, 7.1 Hz, 1H); 2.36 (s, 3H); 2.26-2.19 (m, 1H); 2.09 (dddd, J=12.9, 12.9, 8.3, 6.6 Hz, 1H); 1.74 (dddd, J=13.3, 8.3, 5.4 Hz, 1H); 100 MHz ¹³C NMR (CDCl₃) δ 161.6 (d, J_{C-F}=244 Hz), 138.8 (d, J_{C-F}=8.2 Hz), 137.7, 127.9 (d, J_{C-F-}=8.2 Hz), 117.1 (d, J_{C-F}=20 Hz), 113.1 (d, J_{C-F}=20 Hz), 64.8, 60.8, 49.4, 46.2, 28.9, 19.9.

Intermediate 12:

### 2-(4-Fluoro-2-methyl-phenyl)-3-hydroxymethyl-pyrrolidine-1-carboxylic acid [1-(3,5-bis-trifluoromethyl-phenyl)-ethyl]-methyl-amide

The racemic benzylamine, [1-(3,5-Bis-trifluoromethyl-phenyl)-ethyl]-methyl-amine (0.065 g, 0.221 mmol) was dissolved in 2 mL anhydrous DCE and 0.120 mL (0.845 mmol) of anhydrous Et₃N. Triphosgene (0.021 g, 0.070 mmol) was separately dissolved in DCE and added dropwise to the reaction mixture under N₂. The resulting solution was stirred for 1½ h at rt. Intermediate 11 (0.044 g, 0.211 mmol) was dissolved in fresh DCE and then added to the reaction. The resulting solution was then heated to reflux in an oil bath at 55° C. for 19 h. The reaction was cooled to rt and extracted with saturated aqueous NaHCO₃ (2×10 mL). Combined organics were then dried over Na₂SO₄, filtered and concentrated under reduced pressure. Purification was accomplished by crystallization with hot isopropyl ether to give a colorless solid (0.102 g, 0.202 mmol, 95% yield); Rf 0.3 (50% EtOAc/Hexanes); LRMS m/z (APCI+) 507 [M+H]; 400 MHz ¹HNMR (CDCl₃) δ diagnostic peak of benzylic hydrogen of the racemic side chain at 5.42 (q, J=7.1 Hz, 1H) of trans isomer in 1:1 ratio to the cis isomer; 5.31(q, J=6.9 Hz, 1H).

Intermediate 13:

### 2-(4-Fluoro-2-methyl-phenyl)-3-hydroxymethyl-pyrrolidine-1-carboxylic acid [1-(R)-(3,5-bis-trifluoromethyl-phenyl)-ethyl]-methyl-amide

The benzylamine, [1-(R)-(3,5-Bis-trifluoromethyl-phenyl)-ethyl]-methyl-amine (11.4 g, 42.1 mmol) was dissolved in 100 mL anhydrous DCE and 23.5 mL (168 mmol) of anhydrous Et₃N. Triphosgene (4.1 g, 13.9 mmol) was separately dissolved in DCE and added dropwise to the reaction mixture under N₂. The resulting solution was stirred for 1½ h at rt. Intermediate 11 (2.0 g, 9.6 mmol) was dissolved in fresh DCE and added to the reaction and the solution was heated to reflux in an oil bath at 55° C. for 19 h. The reaction was then cooled to rt and extracted with saturated aqueous NaHCO₃ (2×50 mL). Combined organics were dried over Na₂SO₄, filtered and concentrated under reduced pressure. Purification was accomplished by flash chromatography on a 75S Biotage silica gel column eluting with 50% EtOAc/Hexanes and collecting 25 mL fractions. Product containing fractions were collected and concentrated under reduced pressure to give a colorless solid (17.4 g, 34.5 mmol, 82% yield); Rf 0.3 (50% EtOAc/Hexanes); LRMS m/z (APCI⁺) 507 [M+H]; 400 MHz ¹HNMR (CDCl₃) δ diagnostic peak of benzylic hydrogen of the racemic side chain at 5.42 (q, J=7.1 Hz, 1H) of trans isomer in 1:1 ratio to the cis isomer; 5.31 (q, J=6.9 Hz, 1H).

Diastereomers Separated:

Intermediate 14:

### 2-(S)-(4-Fluoro-2-methyl-phenyl)-3-(S)-hydroxymethyl-pyrrolidine-carboxylic acid [1-(R)-(3,5-bis-trifluoromethyl-phenyl)-ethyl]-methyl-amide

colorless crystalline solid: R, R Whelk O-1 (4.6 mm×25 cm) 1 mL/min, 85/15 Heptane/EtOH, retention time 9.08 min; [α]₂₂^{D}=+5.780 (c 1.00, CH₂Cl₂); 400 MHz ¹HNMR (CD₃OD) δ 7.73 (s, 1H); 7.64 (s, 2H); 7.21 (dd, J=8.3, 5.7 Hz, 1H); 6.89-6.83 (m, 2H); 5.44 (q, J=7.1 Hz, 1H); 5.08 (d, J=7.8 Hz, 1H); 3.76 (ddd, J=9.3, 7.3, 7.3 Hz, 1H); 3.71-3.61 (m, 3H); 2.60 (s, 3H); 2.43 (s, 3H); 2.28 (dddd, J=14.0, 5.7, 5.7, 5.7 Hz, 1H); 2.14 (dddd, J=12.4, 6.7, 6.7, 3.6 Hz, 1H); 1.91-1.85 (m, 2H); 1.50 (d, J=6.7 Hz, 3H).

Intermediate 15:

### 2-(R)-(4-Fluoro-2-methyl-phenyl)-3-(R)-hydroxymethyl-pyrrolidine-1-carboxylic acid [1-(R)-(3,5-bis-trifluoromethyl-phenyl)-ethyl]-methyl-amide colorless crystalline solid

R, R Whelk O-1 (4.6 mm×25 cm) 1 mL/min, 85/15 Heptane/EtOH, retention time 11.09 min; [α]₂₂^{D}=+93.5° (c 1.02, CH₂Cl₂); 500 MHz ¹HNMR (CD₃OD) δ 7.73 (s, 1H); 7.64 (s, 2H); 7.20 (dd, J=8.3, 5.7 Hz, 1H); 6.88-6.82 (m, 2H); 5.43 (q, J=7.1 Hz, 1H); 5.08 (d, J=7.8 Hz, 1H); 3.74 (dddd, J=9.3, 9.3, 9.3, 0.0 Hz, 1H); 3.70-3.60 (m, 2H); 2.60 (s, 3H); 2.42 (s, 3H); 2.27 (dddd, J=14.0, 5.7, 5.7, 5.7 Hz, 1H); 2.19-2.10 (m, 1H); 1.90-1.82 (m, 1H); 1.49 (d, J=7.3 Hz, 3H).

Intermediate 16:

### 2-(R)-(4-Fluoro-2-methyl-phenyl)-1-(toluene-4-sulfonyl)-pyrrolidine-3-carboxylic acid ethyl ester

Intermediate 6 (10.0 g, 24.8 mmol), and 10% Pd/C (1.00 g, 10% weight) were flushed with N₂ and then suspended in EtOH. The reaction was flushed with H₂ and maintained under a H₂ atmosphere at 50 psi for 1 hour. The reaction was then filtered through a plug of celite and concentrated under reduced pressure to give a colorless oil. Purification was accomplished through crystallization with hot isopropyl ether to afford a colorless solid (10.0 g, 24.8 mmol, 100% yield); Rf 0.4 (50% EtOAc/Hexanes); LRMS m/z (APCI⁺) 406 [M+H]; 400 MHz ¹HNMR (CDCl₃) δ diagnostic peak of benzylic hydrogen at 5.28 (d, J=9.5 Hz, 1H) of trans isomer in 3:1 ratio to cis isomer; 5.21 (d, J=2.9 Hz, 1H).

Intermediate 17:

### 2-(R)-(4-Fluoro-2-methyl-phenyl)-1-(toluene-4-sulfonyl)-pyrrolidine-3-(R)-carboxylic acid

Intermediate 16 (10.0 g, 24.7 mmol) was suspended in 250 mL of MeOH. 250 mL of aqueous 1M NaOH was added and the reaction was heated in an oil bath at 40° C. for 18 h. The solvent was removed under reduced pressure and the remaining aqueous layer was then acidified with 1M HCl (250 mL) and extracted with EtOAc (3×100 mL). Combined organics were dried over Na₂SO₄, filtered and concentrated under reduced pressure to give a colorless solid. (9.35 g, 24.7 mmol, 100% yield); Rf 0.5 (10% MeOH/CH₂Cl₂); LRMS m/z (APCI⁺) 376/378 [M −/+H]; 400 MHz ¹HNMR (CD₃OD) δ 7.62 (d, J=8.3 Hz, 2H); 7.27 (dd, J=8.3, 5.8 Hz, 1H); 7.22 (d, J=7.9 Hz, 2H); 6.88-6.80 (m, 2H); 5.27 (d, J=2.9 Hz, 1H); 3.74 (dddd, J=7.5, 7.5, 7.5, 3.7 Hz, 1H); 3.49 (dddd, J=9.5, 7.1, 7.1, 7.1 Hz, 1H); 2.79 (dddd, J=3.3, 3.3, 3.3, 3.3 Hz, 1H); 2.39 (s, 3H); 2.34 (s, 3H); 2.21-2.08 (m, 2H); 100 MHz ¹³C NMR (CD₃OD) δ 178.1, 162.1 (d, J_{C-F}=150 Hz), 143.8, 137.0, 136.9, 135.8, 134.9, 129.6, 128.3, 128.2, 127.8, 117.5 (d, J_{C-F}=20 Hz), 113.1 (d, J_{C-F}=20 Hz), 62.2, 51.8, 48.2, 27.0, 21.8, 19.7.

Intermediate 18:

### [2-(R)-(4-Fluoro-2-methyl-phenyl)-pyrrolidin-3-(R)-yl]-methanol

A 1M solution of Na/Naphthalene was made by dissolving 26.7 g of naphthalene and 3.5 g Na in 150 mL of anhydrous DME and stirring this suspension at rt for 2 days. Intermediate 9 (5.00 g, 13.8 mmol) was separately dissolved in 100 mL of anhydrous DME and cooled under N₂ to −78° C. in a dry ice/acetone bath. The freshly made Na/Naphthalene solution (69.0 mL, 68.9 mmol) was then added portionwise until the dark blue color of reaction remained. The reaction was stirred for another 10 min, then quenched with 10 mL H₂O and allowed to warm to rt. The solution was then concentrated under reduced pressure, redissolved in 1M aqueous HCl and extracted with CH₂Cl₂ (2×40 mL). The aqueous layer was then basified with 1M aqueous NaOH to a pH of 9.0, and extracted with fresh CH₂Cl₂ (3×50 mL). The organic layer was then dried over Na₂SO₄, filtered and concentrated to give a pale yellow oil. Purification was accomplished by flash chromatography on a 40M Biotage silica gel column eluting with a gradient system of 2%, 5%, 10%, 20% MeOH/CH₂Cl₂ collecting 18 mL fractions. Product containing fractions (98-258) were collected and concentrated under reduced pressure to give a pale oil (2.11 g, 10.1 mmol, 73% yield); Rf 0.2 (10% MeOH/CH₂Cl₂); LRMS m/z (APCI+) 210 [M+H]; 400 MHz ¹HNMR (CDCl₃) δ 7.33 (dd, J=8.3, 5.8 Hz, 1H); 6.89-6.81 (m, 2H); 4.13 (d, J=7.1 Hz, 1H); 3.61 (dddd, J=10.4, 10.4, 10.4, 5.8 Hz, 2H); 3.22 (ddd, J=10.3, 8.3, 5.0 Hz, 1H); 3.06 (ddd, J=10.4, 8.7, 7.1 Hz, 1H); 2.36 (s, 3H); 2.26-2.19 (m, 1H); 2.09 (dddd, J=12.9, 12.9, 8.3, 6.6 Hz, 1H); 1.74 (dddd, J=13.3, 8.3, 5.4 Hz, 1H); 100 MHz ¹³C NMR (CDCl₃) δ 161.6 (d, J_{C-F}=244 Hz), 138.8 (d, J_{C-F}=8.2 Hz), 137.7, 127.9 (d, J_{C-F-}=8.2 Hz), 117.1 (d, J_{C-F}=20 Hz), 113.1 (d, J_{C-F}=20 Hz), 64.8, 60.8, 49.4, 46.2, 28.9, 19.9.

Intermediate 19:

### [2-(S)-(4-Fluoro-2-methyl-phenyl)-pyrrolidin-3-(S)-yl]-methanol

A 1M solution of Na/Naphthalene was made by dissolving 26.7 g of naphthalene and 3.5 g Na in 150 mL of anhydrous DME and stirring this suspension at rt for 2 days. Intermediate 10 (4.40 g, 12.1 mmol) was separately dissolved in 50 mL of anhydrous DME and cooled under N₂ to −78° C. in a dry ice/acetone bath. The freshly made Na/Naphthalene solution (60.1 mL, 60.1 mmol) was then added portionwise until the dark blue color of reaction remained. The reaction was stirred for another 10 min, then quenched with 5 mL H₂O and allowed to warm to rt. The solution was then concentrated under reduced pressure, redissolved in 1M aqueous HCl and extracted with CH₂Cl₂ (2×20 mL). The aqueous layer was then basified with 1M aqueous NaOH to a pH of 9.0, and extracted with fresh CH₂Cl₂ (3×30 mL). The organic layer was then dried over Na₂SO₄, filtered and concentrated to give a pale yellow oil (2.35 g, 11.2 mmol, 93% yield); Rf 0.2 (10% MeOH/CH₂Cl₂ LRMS m/z (APCI+) 210 [M+H]; 400 MHz ¹HNMR (CDCl₃) δ 7.33 (dd, J=8.3, 5.8 Hz, 1H); 6.89-6.81 (m, 2H); 4.13 (d, J=7.1 Hz, 1H); 3.61 (dddd, J=10.4, 10.4, 10.4, 5.8 Hz, 2H); 3.22 (ddd, J=10.3, 8.3, 5.0 Hz, 1H); 3.06 (ddd, J=10.4, 8.7, 7.1 Hz, 1H); 2.36 (s, 3H); 2.26-2.19 (m, 1H); 2.09 (dddd, J=12.9, 12.9, 8.3, 6.6 Hz, 1H); 1.74 (dddd, J=13.3, 8.3, 5.4 Hz, 1H); 100 MHz ¹³C NMR (CDCl₃) δ 161.6 (d, J_{C-F}=244 Hz), 138.8 (d, J_{C-F}=8.2 Hz), 137.7, 127.9 (d, J_{C-F}=8.2 Hz), 117.1 (d, J_{C-F}=20 Hz), 113.1 (d, J_{C-F}=20 Hz), 64.8, 60.8, 49.4, 46.2, 28.9, 19.9.

Intermediate 20:

### [1-(S)-(3,5-Bis-trifluoromethyl-phenyl)-ethyl]-methyl-amine

To benzyl amine, [1-(S)-(3,5-Bis-trifluoromethyl-phenyl)-ethyl]-methyl-amine (11.0 g, 0.04 mmol) in 100 mL EtOAc was added R-(−)-malic acid (5.50 g, 0.04 mmol). A precipitate crashed out of the colorless solution after 20 minutes of stirring and then redissolved. After an additional 30 minutes the precipitate crashed out again. After 2 hours, the reaction was cooled to 0° C. in an ice water bath and stirred for an additional 3 h. The suspension was then filtered and washed with 40 mL of EtOAc. The colorless solid was then taken up in 75 mL of hot refluxing EtOAc and then allowed to cool to rt overnight. The solids were filtered and washed with 50 mL EtOAc, followed by 25 mL Hexanes and dried under reduced pressure. The solids were then free-based with 1M NaOH and extracted with EtOAc (2×50 mL), dried over MgSO₄, filtered and concentrated under reduced pressure to give a colorless oil (9.00 g, 33.2 mmol). Purification was accomplished through distillation at 1 mm (oil bath @ 100° C.) to yield a colorless oil (7.80 g, 28.8 mmol, 72% yield); LRMS m/z (APCI⁺) 272 [M+H]; 400 MHz ¹HNMR (CDCl₃) δ 7.81 (s, 2H); 7.76 (s, 1H); 3.80 (q, J=6.6 Hz, 1H); 2.31 (s, 3H); 1.74 (bs, 1H); 1.38 (d, J=6.6 Hz, 3H); [α]₂₀^{D}=−45.2° (c 1.00, CH₂Cl₂).

Intermediate 21:

### 2-(S)-(4-Fluoro-2-methyl-phenyl)-3-(S)-hydroxymethyl-pyrrolidine-1-(S)-carboxylic acid [1-(3,5-bis-trifluoromethyl-phenyl)-ethyl]-methyl-amide

Intermediate 20 (3.0 g, 11.0 mmol) was dissolved in 50 mL anhydrous DCE and anhydrous Et₃N (6.13 mL 44.0 mmol). Triphosgene (1.1 g, 3.6 mmol) was separately dissolved in DCE and added dropwise to the reaction mixture under N₂. The resulting solution was stirred for 1½ h at rt. Intermediate 19 was dissolved in fresh DCE and added to the reaction and the solution was heated to reflux in an oil bath at 55° C. for 19 h. The reaction was then cooled to rt and extracted with saturated aqueous NaHCO₃ (2×25 mL). Combined organics were dried over Na₂SO₄, filtered and concentrated under reduced pressure. Purification was accomplished by flash chromatography on a 75S Biotage silica gel column eluting with a gradient system of 25%, 50% EtOAc/Hexanes and collecting 25 mL fractions. Product containing fractions (8-90) were collected and concentrated under reduced pressure to give a colorless solid (17.4 g, 34.5 mmol, 82% yield); Rf 0.3 (50% EtOAc/Hexanes); LRMS m/z (APCI⁺) 507 [M+H]; 500 MHz ¹HNMR (CDCl₃) δ 7.75 (s, 1H); 7.58 (s, 2H); 7.18 (dd, J=8.3, 5.8 Hz, 1H); 6.86-6.79 (m, 2H); 5.30 (q, J=6.9 Hz, 1H); 5.05 (d, J=7.9 Hz, 1H); 3.76 (dddd, J=9.1, 7.1, 7.1, 0.0 Hz, 1H); 3.69-3.57 (m, 3H); 2.52 (s, 3H); 2.38 (s, 3H); 2.25 (dddd, J=14.9, 6.2, 6.2, 6.2 Hz, 1H); 2.13 (dddd, J=6.6, 6.6, 6.6, 3.3 Hz, 1H); 1.87-1.80 (m, 2H); 1.54 (d, J=6.6 Hz, 3H).

Intermediate 22:

### 2-(R)-(4-Fluoro-2-methyl-phenyl)-3-(R)-hydroxymethyl-pyrrolidine-1-(S)-carboxylic acid [1-(3,5-bis-trifluoromethyl-phenyl)-ethyl]-methyl-amide

Intermediate 20 (2.59 g, 9.56 mmol) was dissolved in 100 mL anhydrous DCE and 5.33 mL (38.20 mmol) of anhydrous Et₃N. Triphosgene (0.94 g, 3.15 mmol) was separately dissolved in DCE and added dropwise to the reaction mixture under N₂. The resulting solution was stirred for 1½ h at rt. Intermediate 18 was dissolved in DCE and added to the reaction and the solution was then heated to reflux in an oil bath at 55° C. for 19 h. The reaction was then cooled to rt and extracted with saturated aqueous NaHCO₃ (2×25 mL). Combined organics were then dried over Na₂SO₄, filtered and concentrated under reduced pressure. Purification was accomplished by flash chromatography on a 40M Biotage silica gel column eluting with a gradient system of 5%, 10% MeOH/CH₂Cl₂ and collecting 18 mL fractions. Product containing fractions (16-30) were collected and concentrated under reduced pressure to give a colorless solid (4.48 g, 8.85 mmol, 93% yield); Rf 0.5 (10% MeOH/CH₂Cl₂); LRMS m/z (APCI⁺) 507 [M+H]; 400 MHz ¹HNMR (CDCl₃) δ 7.71 (s, 1H); 7.62 (s, 2H); 7.19 (dd, J=8.3, 5.8 Hz, 1H); 6.88-6.81 (m, 2H); 5.40 (q, J=6.9 Hz, 1H); 5.06 (d, J=7.9 Hz, 1H); 3.77-3.59 (m, 4H); 2.58 (s, 3H); 2.41 (s, 3H); 2.26 (dddd, J=9.1, 6.2, 6.2, 6.2 Hz, 1H); 2.13 (dddd, J=6.6, 6.6, 6.6, 3.3 Hz, 1H); 1.88-1.83 (m, 1H); 1.67 (bs, 1H); 1.48 (d, J=7.1 Hz, 3H).

Intermediate 23:

### 2-(4-Fluoro-2-methyl-phenyl)-3-formyl-pyrrolidine-1-carboxylic acid [1-(R)-(3,5-bis-trifluoromethyl-phenyl)-ethyl]-methyl-amide

To a solution of intermediate 13 (3.10 g, 6.16 mmol) in 200 mL of anhydrous CH₂Cl₂ in a 0° C. ice water bath was added 4 Å powder (3.10 g, 1/1 weight) and 4-methyl morpholine N-oxide (NMO) (1.08 g, 9.24 mmol). The reaction was stirred under N₂ for ½ hour. Tetra-n-propylammonium penathenate (TPAP) (0.11 g, 0.31 mmol) was then added and the reaction was allowed to warm to rt and stir for an additional hour. The reaction was concentrated under reduced pressure and the residual oil was redissolved in EtOAc and filtered through a plug of silica/celite/MgSO₄. The filtrate was then concentrated under reduced pressure to give a pale brown foam. The crude material was used directly in the reductive amination step.

Intermediate 24:

### 2-(R)-(4-Fluoro-2-methyl-phenyl)-3-(R)-formyl-pyrrolidine-1-carboxylic acid [1-(R)-(3,5-bis-trifluoromethyl-phenyl)-ethyl]-methyl-amide

To a solution of intermediate 15 (5.0 g, 9.9 mmol) in 200 mL of anhydrous CH₂Cl₂ in a 0° C. ice water bath was added 4 Å powder (5.0 g, 1/1 weight) and 4-methyl morpholine N-oxide (1.7 g, 15 mmol). The reaction was stirred under N₂ for ½ hour. TPAP (0.173 g, 0.49 mmol) was then added and the reaction was allowed to warm to rt and stir for an additional hour. The reaction was concentrated under reduced pressure and the residual oil was redissolved in EtOAc and filtered through a plug of silica/celite/MgSO₄. The filtrate was then concentrated under reduced pressure to give a pale brown foam. The crude material was used directly in the reductive amination step.

Intermediate 25:

### 2-(R)-(4-Fluoro-2-methyl-phenyl)-3-(R)-formyl-pyrrolidine-1-carboxylic acid [1-(S)-(3,5-bis-trifluoromethyl-phenyl)-ethyl]-methyl-amide

To a solution of intermediate 22 (2.97 g, 5.90 mmol) in 100 mL of anhydrous CH₂Cl₂ in a 0° C. ice water bath was added 4 Å powder (3.00 g, 1/1 weight) and 4-methyl morpholine N-oxide (1.03 g, 8.80 mmol). The reaction was stirred under N₂ for ½ hour. TPAP (0.10 g, 0.29 mmol) was then added and the reaction was allowed to warm to rt and stir for an additional hour. The reaction was concentrated under reduced pressure and the residual oil was redissolved in EtOAc and filtered through a plug of silica/celite/MgSO₄. The filtrate was then concentrated under reduced pressure to give a pale brown foam. The crude material was used directly in the reductive amination step.

Intermediate 26:

### 2-(S)-(4-Fluoro-2-methyl-phenyl)-3-(S)-formyl-pyrrolidine-1-carboxylic acid [1-(S)-(3,5-bis-trifluoromethyl-phenyl)-ethyl]-methyl-amide

To a solution of intermediate 21 (1.50 g, 2.96 mmol) in 100 mL of anhydrous CH₂Cl₂ in a 0° C. ice water bath was added 4 Å powder (1.50 g, 1/1 weight) and 4-methyl morpholine N-oxide (0.52 g, 4.44 mmol). The reaction was stirred under N₂ for ½ hour. TPAP (0.05 g, 0.15 mmol) was then added and the reaction was allowed to warm to rt and stir for an additional hour. The reaction was concentrated under reduced pressure and the residual oil was redissolved in EtOAc and filtered through a plug of silica/celite/MgSO₄. The filtrate was then concentrated under reduced pressure to give a pale brown foam. The crude material was used directly in the reductive amination step.

Intermediate 27:

### 2-(S)-(4-Fluoro-2-methyl-phenyl)-3-(S)-formyl-pyrrolidine-1-carboxylic acid [1-(R)-(3,5-bis-trifluoromethyl-phenyl)-ethyl]-methyl-amide

To a solution of intermediate 14 (0.50 g, 0.99 mmol) in 30 mL of anhydrous CH₂Cl₂ in a 0° C. ice water bath was added 4 Å powder (0.50 g, 1/1 weight) and 4-methyl morpholine N-oxide (0.17 g, 1.48 mmol). The reaction was stirred under N₂ for ½ hour. TPAP (20.0 mg, 0.05 mmol) was then added and the reaction was allowed to warm to rt and stir for an additional hour. The reaction was concentrated under reduced pressure and the residual oil was redissolved in EtOAc and filtered through a plug of silica/celite/MgSO₄. The filtrate was then concentrated under reduced pressure to give a pale brown foam. The crude material was used directly in the reductive amination step.

Intermediate 28:

### (2-Bromo-pyridin-3-yl)-carbamic acid tert-butyl ester

To the known starting material 2-bromo-nicotinic acid (J. Org. Chem.; 14; 1949; 509, 513) (3.0 g, 13.0 mmol) in 26 mL of t-BuOH was added diphenylphosporyl azide (3.9 g, 14.3 mmol). The resulting solution was heated in an oil bath at 80° C. for 3 h. Upon cooling, the reaction solution was then concentrated and rediluted in EtOAc and washed with H₂O (50 mL), followed by saturated aqueous NaHCO₃ (30 mL) and brine (30 mL). Combined organics were then dried over Na₂SO₄, filtered and concentrated under reduced pressure. Purification was accomplished by flash chromatography on a 35L Biotage silica gel column eluting with 20% EtOAc/Hexanes and collecting 18 mL fractions. Product containing fractions (19-29) were combined and concentrated under reduced pressure to give colorless oil (3.5 g, 12.8 mmol, 99% yield); Rf 0.75 (20% EtOAc/Hexanes); LRMS m/z (APCI⁺) 274/275 [M+H]; 400 MHz ¹HNMR (CDCl₃) δ 8.44 (dd, J=7.9, 1.5 Hz, 1H); 8.02 (dd, J=4.6, 1.7 Hz, 1H); 7.23 (dd, J=8.3, 4.6 Hz, 1H); 7.03 (bs, 1H); 1.53 (s, 9H).

Intermediate 29:

### [2-(4-Fluoro-2-methyl-phenyl)-pyridin-3-yl]-carbamic acid tert-butyl ester

Intermediate 28 (1.67 g, 6.13 mmol) was dissolved in 15 mL of anhydrous DME under N₂. Pd(Ph₃P)₄ (0.08 g, 5% by weight) catalyst was then added and the reaction was stirred for ½ h at rt. The colorless solution turns from yellow to orange and back to yellow. Intermediate 1 (1.59 g, 6.74 mmol) was then added, followed by 18.5 mL of 1M K₂CO₃ and the biphasic solution was heated to reflux for 2 h. The resulting green suspension was cooled to rt, diluted with EtOAc (50 mL) and washed with saturated aqueous NaHCO₃. Combined organics were then washed with brine, dried over Na₂SO₄, filtered and concentrated under reduced pressure to give a pale green solid (2.51 g). Purification was accomplished by flash chromatography on a 40M Biotage silica gel column using a gradient of 10%, 20%, 30% EtOAc/Hexanes and collecting 18 mL factions. Product containing fractions (31-50) were combined and concentrated under reduced pressure to give a colorless crystalline solid (1.42 g, 4.70 mmol, 77% yield); Rf 0.5 (25% EtOAc/Hexanes); LRMS m/z (APCI⁺) 301/303 [M−/+H]; 400 MHz ¹HNMR (CDCl₃) δ 8.59 (d, J=8.3 Hz, 1H); 8.34 (dd, J=5.0, 1.7 Hz, 1H); 7.30-7.22 (m, 2H); 7.04 (ddd, J=17.4, 9.5, 2.5 Hz, 2H); 6.14 (bs); 2.13 (s, 3H); 1.46 (s, 9H); 100 MHz;. ¹³C NMR (CDCl₃) δ 192.9, 162.0, 152.8, 143.7, 133.5, 131.4, 131.3, 126.6, 123.3, 118.0, 117.8, 113.9, 113.7, 81.6, 45.2, 28.4, 19.7.

Intermediate 30:

### [2-(4-Fluoro-2-methyl-phenyl)-piperidin-3-yl]-carbamic acid tert-butyl ester

Intermediate 29 (1.40 g, 4.63 mmol) was dissolved in 20/25 mL of a 1/1.25 solution of EtOH/AcOH and flushed with N₂. PtO₂ (0.28 g, 20% by weight) was added and the reaction was flushed with H₂ and maintained under a H₂ atmosphere at 45 psi for 2 h. The reaction was filtered through a plug of celite and concentrated under reduced pressure. The residual oil was then redissolved in EtOAc (50 mL) and H₂O (30 mL). The aqueous layer was basified to pH 8.0 with saturated NaHCO₃ and extracted. Combined organics were then washed with brine, dried over Na₂SO₄, filtered and concentrated to give a tan oil (2.27 g). Purification was accomplished by flash chromatography on a 40M Biotage silica gel column using a gradient system of 0%, 5%, 7%, 10% MeOH/CH₂Cl₂ and collecting 18 mL fractions. Product containing fractions (60-86) were combined and concentrated to give a colorless foam. This material was then recrystallized from hot 25% EtOAc/Hexanes to give a colorless solid (1.20 g, 3.90 mmol, 84% yield); Rf 0.5 (10% MeOH/CH₂Cl₂); LRMS m/z (APCI⁺) 309 [M+H].

Intermediate 31:

### [1-{[1-(3,5-Bis-trifluoromethyl-phenyl)-ethyl]-methyl-carbamoyl}-2-(4-fluoro-2-methyl-phenyl)-piperidin-3-yl]-carbamic acid tert-butyl ester

To intermediate 30 (0.95 g, 3.08 mmol) in 10 mL of anhydrous CH₂Cl₂ is added 1.68 mL of Et₃N (12.00 mmol). Triphosgene (0.30 g, 1.02 mmol) was separately dissolved in CH₂Cl₂ and added dropwise to the reaction under N₂. The resulting solution was stirred for 1½ h. The racemic form of intermediate 20 (0.95 g, 3.08 mmol) and 0.72 mL Hunig's base (DIPEA) were then added and the reaction was heated to reflux in an oil bath at 45° C. for 22 h. The reaction was cooled to rt and was suspended in 1M aqueous HCl (30 mL) and extracted with CH₂Cl₂ (2×20 mL). Combined organics were then washed with brine, dried over Na₂SO₄, filtered and concentrated under reduced pressure to give a pale foam (2.02 g). Purification was accomplished by flash chromatography on a 40M Biotage silica gel column using a gradient system of 2%, 4%, 6% MeOH/CH₂Cl₂ and collecting 18 mL fractions. Product containing fractions (70-76) were combined and concentrated under reduced pressure to give 1.12 g colorless foam (1.85 mmol, 60% yield). 1:1 mixture of cis:trans isomers.

Diastereomers Separated:

Intermediate 32:

### [1-{[1-(3,5-Bis-trifluoromethyl-phenyl)-ethyl]-methyl-carbamoyl}-2-(4-fluoro-2-methyl-phenyl)-piperidin-3-yl]-carbamic acid tert-butyl ester

(less polar isomer, racemic) Purification was accomplished by flash chromatography on a 40M Biotage silica gel column using a gradient system of 30%, 40%, 50%, 60% EtOAc/Hexanes and collected 13 mm fractions. Product containing fractions (42-64) were combined and concentrated to give a colorless foam (0.271 g, 0.45 mmol, 15% yield); Rf 0.80 (10% MeOH/CH₂Cl₂); LRMS m/z (APCI⁺) 606 [M+H].

Intermediate 33:

### [1-{[1-(3,5-Bis-trifluoromethyl-phenyl)-ethyl]-methyl-carbamoyl}-2-(4-fluoro-2-methyl-phenyl)-piperidin-3-yl]-carbamic acid tert-butyl ester

(more polar isomer, racemic) Purification was accomplished by flash chromatography on a 40M Biotage silica gel column using a gradient system of 30%, 40%, 50%, 60% EtOAc/Hexanes and collecting 13 mm fractions. Product containing fractions (82-118) were combined and concentrated under reduced pressure to give a colorless foam (0.290 g, 0.48 mmol, 16% yield); Rf 0.70 (10% MeOH/CH₂Cl₂); LRMS m/z (APCI⁺) 606 [M+H]; 400 MHz ¹HNMR (CDCl₃) δ 7.76 (s, 1H); 7.54 (s, 2H); 6.94-6.90 (m, 1H); 6.86 (dd, J=10.0, 2.9 Hz, 1H); 6.74-6.70 (m, 1H); 5.63 (q, J=7.1 Hz, 1H); 4.49 (bs, 1H); 4.00 (bs, 1H); 3.32 (dd, J=10.8, 0.0 Hz, 1H); 2.86 (s, 3H); 2.71 (s, 3H); 2.04-2.00 (m, 1H); 1.89-1.68 (m, 4H); 1.52 (d, J=7.1 Hz, 3H); 1.25 (s, 9H).

Intermediate 34:

### 2-(4-Fluoro-2-methyl-phenyl)-nicotinic acid ethyl ester

Ethyl-2-chloro-nicotinate (1.00 g, 5.48 mmol) and intermediate 1 (1.53 g, 7.47 mmol) were dissolved in 66.0 mL of anhydrous DCE and 22.0 mL (21.6 mmol) of 1.0 M K₂CO₃ aqueous solution was added, followed by Pd(Ph₃)₄ (0.32 g, 0.27 mmol). The resulting solution was heated in an oil bath at 90° C. for 1½ h. The reaction was then allowed to cool to rt overnight. The crude material was dissolved in EtOAc and washed with saturated NaHCO₃ (3×25 mL) aqueous solution. Combined organics were then dried over MgSO4, filtered and concentrated under reduced pressure. Purification was accomplished through flash chromatography on a 35 g Isco silica gel column eluting with 20% EtOAc/Hexanes and collecting 18 mL fractions. Product containing fractions were combined and concentrated under reduced pressure to give the product as a yellow viscous oil (1.20 g, 4.63 mmol, 91% yield); Rf 0.4 (25% EtOAc/Hexanes); LRMS m/z (APCI⁺) 260 [M+H]; 400 MHz ¹HNMR (CDCl₃) δ 8.76-8.74 (m, 1H); 8.24 (dd, J=8.3, 2.1 Hz, 1H); 7.39-7.35 (m, 1H); 7.09 (ddd, J=7.9, 5.8, 2.1 Hz, 1H); 6.95-6.87 (m, 2H); 4.07 (q, J=7.2 Hz, 2H); 2.09 (s, 3H); 1.00 (ddd, J=7.1, 7.1, 2.5 Hz, 3H).

Intermediate 35:

### 2-(2,4-Dimethyl-phenyl)-piperidine-3-carboxylic acid ethyl ester

Intermediate 34 (5.5 g, 21.2 mmol) was dissolved in 100 mL of EtOH and flushed with N₂. PtO₂ (96.0 mg, 4.2 mmol) was then added and the reaction was subjected to H₂ at 50 psi. After 4 hours, 40 mL of TFA was added and the reaction was resubjected to H₂ at 50 psi for an additional ½ h. The reaction solution was then filtered through a plug of celite and the filtrate was concentrated under reduced pressure. The residual oil was partitioned between CH₂Cl₂ and saturated NaHCO₃ aqueous solution. The organics were extracted and dried over MgSO₄, filtered and concentrated under reduced pressure. Purification was accomplished through flash chromatography on a 40M Biotage silica gel column eluting with a gradient system of 50-90% EtOAc/Hexanes and collecting 18 mL fractions. Product containing fractions were combined and concentrated under reduced pressure to give the product as a colorless oil (4.3 g, 16.3 mmol, 76% yield); Rf 0.1 (50% EtOAc/Hexanes); LRMS m/z (APCI⁺) 266 [M+H]; 400 MHz ¹HNMR (CDCl₃) δ 7.21 (dd, J=9.5, 6.2 Hz, 1H); 6.83-6.79 (m, 2H); 4.05 (d, J=3.3 Hz, 1H); 3.92-3.82 (m, 2H); 3.36 (dd, J=13.3, 0.0 Hz, 1H); 2.86 (ddd, J=12.4, 2.9, 0.0 Hz, 1H); 2.81 (dd, J=2.9, 2.9 Hz, 1H); 2.31 (d, J=2.5 Hz, 3H); 2.14-2.11 (m, 1H); 2.01-1.92 (m, 1H); 1.91-1.86 (m, 2H); 1.55-1.51 (m, 1H); 0.96 (t, J=7.3 Hz, 3H); 100 MHz ¹³C NMR (CDCl₃) δ 173.6, 161.7 (d, J_{C-F}=240 Hz), 137.1, 135.6, 127.5, 117.0 (d, J_{C-F}=20 Hz), 112.6 (d, J_{C-F}=20 Hz), 60.2, 58.5, 47.7, 47.5, 42.6, 27.8, 21.7, 19.5, 14.1.

Intermediate 36:

### [2-(4-Fluoro-2-methyl-phenyl)-piperidin-3-yl]-methanol

Intermediate 35 (4.3 g, 16.2 mmol) was dissolved in a 1/1 solution of EtOH/H₂O (100 mL) and solid NaOH pellets (6.5 g, 162.3 mmol) were added. The resulting solution was heated in an oil bath at 85° C. for 16 h. The reaction was then cooled and concentrated under reduced pressure, azeotroping the water off with isopropanol. The crude solids were then stirred in an isopropanol/isopropyl ether solution for ½ h. The brown solids were filtered and dried to give clean product. The filtrate was then reconcentrated and also subjected to isopropanol/isopropyl ether solution for ½ h. HCl was added until the pH of the solution was 5.0. The HCl salt formed and was filtered and dried to give additional product. A total of 13.1 g of crude product was recovered (53.2 mmol). This material was carried on to the next step without further purification.

The crude acid (3.5 g, 12.8 mmol) was dissolved in 100 mL of anhydrous THF and cooled to 0° C. under N₂. 1.0 M LAH in THF (19.2 mL, 19.2 mmol) was then added dropwise and the resulting reaction was allowed to warm to rt and stirred for 1 h. The reaction was quenched with H₂O and 15% NaOH and then filtered through a plug of celite/MgSO₄. The filtrate was concentrated under reduced pressure to give the desired product as a colorless oil (1.8 g, 8.1 mmol, 63% yield); LRMS m/z (APCI⁺) 224 [M+H]; 400 MHz ¹HNMR (CDCl₃) 1:1 mixture of cis:trans isomers.

Intermediate 37:

### 2-(R)-(4-Fluoro-2-methyl-phenyl)-3-(R)-hydroxymethyl-piperidine-1-carboxylic acid [1-(R)-(3,5-bis-trifluoromethyl-phenyl)-ethyl]-methyl-amide (less polar, trans)

Intermediate 38:

2-(4-Fluoro-2-methyl-phenyl)-3-hydroxymethyl-piperidine-1-carboxylic acid [1-(3,5-bis-trifluoromethyl-phenyl)-ethyl]-methyl-amide (more polar, cis)

The benzylamine [1-(R)-(3,5-Bis-trifluoromethyl-phenyl) ethyl]-methyl-amine (2.19 g, 8.07 mmol) was dissolved in 80 mL of anhydrous DCE and Et₃N (4.50 mL, 32.28 mmol) was added. Triphosgene (0.79 g, 2.66 mmol) was separately dissolved in 1 mL of DCE and added dropwise to the reaction. The resulting solution was stirred at rt for 1½ h. Intermediate 36 (1.80 g, 8.07 mmol) was then separately dissolved in another 5 mL of DCE and added to the reaction. The resulting solution was then heated in an oil bath at 65° C. for 16 h. The reaction was then cooled to rt and quenched with saturated NaHCO₃ aqueous solution and extracted with EtOAc (3×25 mL). The combined organics were then dried over MgSO₄, filtered and concentrated under reduced pressure. Purification was accomplished through flash chromatography on a 75S Biotage silica gel column eluting with a gradient system of 10-25% acetone/hexanes and collecting 18 mL fractions. The two diastereomers were isolated. Intermediate 37: The less polar, trans isomer was recovered as a colorless solid (0.19 mg, 0.37 mmol, 5% yield); LRMS m/z (APCI⁺) 521 [M+H]; 400 MHz ¹HNMR (CDCl₃) δ 7.77 (s, 1H); 7.58 (s, 2H); 7.26-7.22 (m, 1H); 6.82 (dd, J=10.0, 2.9 Hz, 1H); 6.77 (ddd, J=8.3, 8.3, 2.5 Hz, 1H); 5.46 (q, J=6.9 Hz, 1H); 4.35 (d, J=9.1 Hz, 1H); 3.34 (ddd, J=44.0, 10.8, 5.0 Hz, 2H); 3.20 (ddd, J=12.0, 3.7, 3.7 Hz, 1H); 2.93 (ddd, J=13.3, 10.4, 2.9 Hz, 1H); 2.63 (s, 3H); 2.43 (s, 3H); 2.08-2.04 (m, 2H); 1.89-1.81 (m, 2H); 1.72-1.69 (m, 1H); 1.41 (d, J=7.1 Hz, 3H). Intermediate 38: The more polar, cis isomer was recovered as a pale yellow solid; LRMS m/z (APCI⁺) 521 [M+H]; 400 MHz ¹HNMR (CDCl₃) δ 7.76 (s, 1H); 7.66 (s, 2H); 7.15 (dd, J=8.7, 5.8 Hz, 1H); 6.87 (dd, J=10.0, 2.9 Hz, 1H); 6.76 (ddd, J=8.3, 8.3, 2.5 Hz, 1H); 5.54 (q, J=7.1 Hz, 1H); 4.92 (d, J=5.0 Hz, 1H); 3.56 (ddd, J=29.7, 10.8, 7.5 Hz, 2H); 3.25 (dddd, J=6.6, 6.6, 3.3, 3.3 Hz, 1H); 3.01 (ddd, J=12.0, 8.3, 3.3 Hz, 1H); 2.70 (s, 3H); 2.35 (s, 3H); 2.22-2.19 (m, 1H); 2.06-2.03 (m, 1H); 1.87 (dddd, J=8.3, 8.3, 8.3, 4.6 Hz, 1H); 1.78-1.72 (m, 1H); 1.64-1.60 (m, 1H); 1.53 (d, J=7.5 Hz, 3H).

Intermediate 39;

### 2-(R)-(4-Fluoro-2-methyl-phenyl)-3-(R)-formyl-piperidine-1-carboxylic acid [1-(R)-(3,5-bis-trifluoromethyl-phenyl)-ethyl]-methyl-amide

Intermediate 37 (0.162 g, 0.321 mmol) was dissolved in 6.0 mL of anhydrous CH₂Cl₂ and 4 Å powder (0.162 g, 1/1 weight) and 4-methyl morpholine N-oxide (40.0 mg, 0.34 mmol) was added. The reaction was stirred under N₂ for ½ h. TPAP (5.5 mg, 0.02 mmol) was then added and the reaction was stirred for an additional hour and then concentrated under reduced pressure. The residual oil was then redissolved in EtOAc and filtered through a plug of silica/celite/MgSO₄. The filtrate was concentrated under reduced pressure to give a pale brown foam (0.150 g, 0.290 mmol, 93% yield). The crude material was used directly in the reductive amination step.

Intermediate 40:

### (2-(S)-Phenyl-piperidin-3-(S)-yl)-carbamic acid tert-butyl ester

Cis-2-(S)-phenyl-piperidin-3-(S)-ylamine (1.60 g, 0.91 mmol) was dissolved in 50 mL of acetonitrile and (Boc)₂O (1.98 g, 0.91 mmol) was added. The reaction was stirred at rt under N₂ for 1 h. The solution was then concentrated under reduced pressure to give a colorless solid. The crude material was partitioned between EtOAc and H₂O and the aqueous layer was basified to pH of 8.0 with 1N NaOH. The aqueous layer was extracted with EtOAc several times and the combined organics were then dried over Na₂SO₄, filtered and concentrated under reduced pressure. Purification was accomplished through flash chromatography on a 40M Biotage column eluting with a gradient system of 2%, 5%, 10% MeOH/CH₂Cl₂ and collecting 25 mL fractions. Product containing fractions (21-34) were combined and concentrated under reduced pressure to give the product as a colorless solid (1.06 g, 0.38 mmol, 42% yield); Rf 0.7 (10% MeOH/CH₂Cl₂); LRMS m/z (APCI⁺) 277 [M+H]; 400 MHz ¹HNMR (CDCl₃) δ 7.33-7.26 (m, 4H); 7.23-7.19 (m, 1H); 5.40 (d, J=9.1 Hz, 1H); 3.90 (dd, J=9.1, 0.0 Hz, 1H); 3.84 (s, 1H); 3.17 (ddd, J=11.2, 1.7, 1.7 Hz, 1H); 2.79 (ddd, J=11.2, 2.9, 2.9 Hz, 1H); 1.98-1.53 (m, 4H); 1.24 (s, 9H).

Intermediate 41

### (1-{[1-(3,5-Bis-trifluoromethyl-phenyl)-ethyl]-methyl-carbamoyl}-2-(S)-phenyl-piperidin-3-(S)-yl)-carbamic acid tert-butyl ester (less polar, more active)

Intermediate 42

### (1-{[1-(3,5-Bis-trifluoromethyl-phenyl)-ethyl]-methyl-carbamoyl}-2-(S)-phenyl-piperidin-3-(S)-yl)-carbamic acid tert-butyl ester (more polar)

Intermediate 40 (0.20 g, 0.72 mmol) was dissolved in 7.2 mL of anhydrous CH₂Cl₂ and Et₃N (0.29 g, 2.82 mmol) was added. Triphosgene (0.07 g, 0.23 mmol) was separately dissolved in 1.0 mL of CH₂Cl₂ and added dropwise to the reaction. The resulting solution was stirred at rt for 1½ h. In a separate flask, the racemic form of the known benzyl amine [1-(3,5-Bis-trifluoromethyl-phenyl)-ehtyl]-methyl-amine [WO 01/25219A2] (0.22 g, 0.72 mmol) and DIPEA (0.13 g, 0.97 mmol) were dissolved in 5.0 mL of CH₂Cl₂ and stirred at rt for 1½ h. This latter material was added to the first reaction flask and the resulting solution was heated in an oil bath at 50° C. for 16 h. The reaction was then cooled to rt and diluted with additional CH₂Cl₂ and washed with 1 N HCl (20 mL), followed by brine. The organics were then dried over MgSO₄, filtered and cocentrated under reduced pressure. Purification was accomplished through flash chromatography on a 10 g Isco silica gel column eluting with 30% EtOAc/Hexanes and collecting 8 mL fractions. Product containing fractions were combined and concentrated under reduced pressure. The two diastereomers were isolated. Intermediate 41: Less polar isomer: Rf 0.5 (40% EtOAc/Hexanes); LRMS m/z (APCI⁺) 574 [M+H]. Intermediate 42: More polar isomer: Rf 0.4 (40% EtOAc/Hexanes); LRMS m/z (APCI⁺) 574 [M+H].

Intermediate 43

### [1-(3,5-Bis-trifluoromethyl-benzylcarbamoyl)-2-(S)-phenyl-piperidin-3-(S)-yl]-carbamic acid tert-butyl ester

Intermediate 40 (2.0 g, 7.3 mmol) was dissolved in 72.0 mL of anhydrous CH₂Cl₂ and Et₃N (3.9 mL, 28.3 mmol) was added. Triphosgene (0.7 g, 0.3 mmol) was separately dissolved in 1.0 mL of CH₂Cl₂ and added to the reaction dropwise. The resulting solution was stirred at rt for 1½ h. In a separate flask, 3,5-Bis-trifluoromethyl-benzylamine (1.8 g, 1.3 mmol) and DIPEA (1.7 mL, 9.7 mmol) are dissolved in 5 mL of CH₂Cl₂ and stirred at rt for 1½ h. The latter material was added to the first reaction flask and the resulting solution was heated in an oil bath at 50° C. for 16 h. The reaction was then cooled and washed with 1N HCl, follwed by brine. The organics were then dried over MgSO₄, filtered and concentrated under reduced pressure. Purification was accomplished through flash chromatography on a 40 M Biotage silica gel column eluting with a gradient system of 25-40% EtOAc/Hexanes and collecting 18 mL fractions. Product containing fractions were combined and concentrated under reduced pressure to give the desired product as a colorless solid (2.7 g, 5.0 mmol, 68% yield); LRMS m/z (APCI⁺) 546 [M+H].

Intermediate 44:

### (2-(R)-Phenyl-piperidin-3-(R)-yl)-carbamic acid tert-butyl ester

Cis-2-(R)-phenyl-piperidin-3-(R)-ylamine (0.80 g, 4.55 mmol) was dissolved in 23 mL of acetonitrile and (Boc)₂O (0.99 g, 4.55 mmol) was added. The reaction was stirred at rt under N₂ for 2 hr. The solution was concentrated under reduced pressure to give a colorless oil. The crude material was then purified through flash chromatography on a 10 g Isco silica gel column using a gradient system of 4-6% MeOH/CH₂Cl₂ and collecting 8 mL fractions. Product containing fractions were combined and concentrated under reduced pressure to give the desired product as a white solid (0.70 g, 2.54 mmol, 56% yield); Rf 0.5 (10% MeOH/CH₂Cl₂); LRMS m/z (APCI⁺) 277 [M+H]; 400 MHz ¹HNMR (CDCl₃) δ 7.29-7.23 (m, 4H); 7.20-7.16 (m, 1H); 5.42 (d, J=9.1 Hz, 1H); 3.87 (dd, J=9.1, 0.0 Hz, 1H); 3.80 (s, 1H); 3.13 (ddd, J=10.4, 2.9, 0.0 Hz, 1H); 2.75 (ddd, J=11.2, 11.2, 2.5 Hz, 1H); 1.95-1.90 (m, 1H); 1.71-1.61 (m, 3H); 1.21 (s, 9H).

Intermediate 45:

### (1-{[1-(R)-(3,5-Bis-trifluoromethyl-phenyl)-ethyl]-methyl-carbamoyl}-2-(R)-phenyl-piperidin-3-yl)-carbamic acid tert-butyl ester

Using intermediate 44, the same procedure was followed as for intermediate 41. The crude mixture of diastereomers was purified through flash chromatography using a 35 g Isco silica gel column eluting with 20% EtOAc/Hexanes and collecting 25 mL fractions. The two diastereomers were successfully isolated, and concentrated under reduced pressure. Intermediate 45: (Less polar isomer): (0.16 g, 0.27 mmol, 11% yield); Rf 0.6 (40% EtOAc/Hexanes); LRMS m/z (APCI⁺) 575 [M+H]. Intermediate 46: (more polar isomer): (0.18 g, 0.32 mmol, 13% yield); Rf 0.5 (40% EtOAc/Hexanes); MS 574 [M+H].

Intermediate 47:

### Trans-(2-Phenyl-piperidin-3-yl)-carbamic acid tert-butyl ester

Using a racemic mixture of the trans isomers of 2-phenyl-piperidin-3-ylamine, the same procedure was followed as for intermediate 40. The desired product was obtained as a colorless solid (2.42 g, 8.77 mmol, 30% yield); Rf 0.2 (10% MeOH/CH₂Cl₂); LRMS m/z (APCI⁺) 277 [M+H]. The mixture of isomers was used directly to couple with the racemic 3,5-bis-trifluoromethyl-phenyl side chain, following the same procedure as used for intermediate 41. The crude product was purified via flash chromatography on a 40 M Biotage silica gel column eluting with a gradient system of 15-30% EtOAc/Hexanes and collecting 25 mL fractions. The two isomers were separated in this fashion.

Intermediate 48:

### Trans-(1-{[1-(3,5-Bis-trifluoromethyl-phenyl)-ethyl]-methyl-carbamoyl}-2-phenyl-piperidin-3-yl)-carbamic acid tert-butyl ester (less polar)

(0.26 g, 0.45 mmol, 5% yield); Rf 0.5 (40% EtOAc/Hexanes); LRMS m/z (APCI⁺) 574 [M+H]; 400 MHz ¹HNMR (CDCl₃) δ 7.74 (s, 1H); 7.73 (s, 2H); 7.40-7.31 (m, 4H); 7.24 (dd, J=12.0, 5.0 Hz, 1H); 5.52 (d, J=7.1 Hz, 1H); 5.42 (q, J=6.6 Hz, 1H); 4.85 (s, 1H); 4.32-4.27 (m, 1H); 3.61 (dd, J=12.9, 0.0 Hz, 1H); 3.09 (ddd, J=14.1, 11.6, 3.3 Hz, 1H); 2.57 (s, 3H); 1.89-1.80 (m, 1H); 1.71-1.69 (m, 2H); 1.54 (d, J=7.1 Hz, 3H); 1.40 (s, 9H).

Intermediate 49:

### (1-{[1-(3,5-Bis-trifluoromethyl-phenyl)-ethyl]-methyl-carbamoyl}-2-phenyl-piperidin-3-yl)-carbamic acid tert-butyl ester (more polar)

(0.15 g, 0.26 mmol, 3% yield); Rf 0.4 (40% EtOAc/Hexanes); LRMS m/z (APCI⁺) 574 [M+H]; 400 MHz ¹HNMR (CDCl₃) δ 7.75 (s, 1H); 7.68 (s, 2H); 7.33-7.27 (m, 4H); 7.26-7.21 (m, 1H); 5.55 (d, J=5.8 Hz, 1H); 5.31 (q, J=6.2 Hz, 1H); 4.76 (s, 1H); 4.22 (bs, 1H); 3.40-3.37 (m, 1H); 3.19-3.11 (m, 1H); 2.71 (s, 3H); 1.85-1.73 (m, 3H); -1.55 (d, J=7.1 Hz, 3H); 1.37 (s, 9H).

Intermediate 50:

### Trans-(1-{[1-(3,5-Bis-trifluoromethyl-phenyl)-ethyl]-methyl-carbamoyl}-2-phenyl-piperidin-3-yl)-methyl-carbamic acid tert-butyl ester (less polar isomer)

Intermediate 48 (0.20 g, 0.34 mmol) was dissolved in 3.40 mL of anhydrous THF and (0.24 g, 1.70 mmol) MeI was added followed by (0.04 g, 1.70 mmol) of solid NaOH. Four drops of MeOH was added to help catalyze the reaction. The solution was then stirred at rt under N₂ for 16 h. The reaction was partitioned between EtOAc (10 mL) and saturated aqueous NaHCO₃ (15 mL) and extracted. The combined organics were dried over Na₂SO₄, filtered and concentrated under reduced pressure to give a tan oil. This material was purified through flash chromatography on a 15 g Isco silica gel column eluting with 20% EtOAc/Hexanes and collecting 8 mL fractions. Product containing fractions were then combined and concentrated under reduced pressure to give the desired material as a colorless gum (0.15 g, 0.25 mmol, 74% yield); Rf 0.8 (50% EtOAc/Hexanes); LRMS m/z (APCI⁺) 588 [M+H]; 400 MHz ¹HNMR (CDCl₃) δ 7.74 (s, 1H); 7.55 (d, J=2.9 Hz, 2H); 7.30-7.26 (m, 1H); 7.22-7.12 (m, 4H); 5.54 (s, 1H); 4.12-4.05 (m, 1H); 3.16 (ddd, J=9.5, 9.5, 0.0 Hz, 1H); 2.81-2.66 (m, 8H); 1.87-1.71 (m, 4H); 1.34 (dd, J=7.3, 0.0 Hz, 3H); 1.15 (s, 9H).

Intermediate 51:

### (1-{[1-(3,5-Bis-trifluoromethyl-phenyl)-ethyl]-methyl-carbamoyl}-2-phenyl-piperidin-3-yl)-methyl-carbamic acid tert-butyl ester (more polar isomer)

Using intermediate 49, the same procedure was followed as for intermediate 50. The product was obatined as a colorless foam (0.10 g, 0.17 mmol, 100% yield); Rf 0.6 (50% EtOAc/Hexanes); LRMS m/z (APCI⁺) 488/588 [M+H]; 400 MHz ¹HNMR (CDCl₃) δ 7.69 (s, 1H); 7.38 (d, J=2.1 Hz, 2H); 7.28-7.21 (m, 1H); 7.20-7.13 (m, 4H); 5.57 (q, J=7.1 Hz, 1H); 4.13-4.08 (m, 1H); 3.23 (dd, J=12.0, 12.0, 0.0 Hz, 1H); 2.81-2.66 (m, 8H); 1.88-1.74 (m, 4H); 1.46 (dd, J=6.6, 0.0 Hz, 3H); 1.16 (s, 9H).

Intermediate 52:

### Trans-5-Nitro-6-o-tolyl-piperidin-2-one

Methyl-4-nitrobutyrate (25.0 g, 169.9 mmol) and 16.4 mL of tolualdehyde (17.0 g, 141.6 mmol) were dissolved in 160.0 mL of ethanol and (21.8 g, 283.2 mmol) of ammonium acetate was added and the resulting solution was heated at 85° C. for 16 h. Once cooled, the reaction was concentrated under reduced pressure to ¼ it's volume and isopropyl ether was added to precipitate the product. The precipitate was then redissolved in CH₂Cl₂ (50 mL) and washed with H₂O (50 mL), followed by brine (50 mL). The organics were then dried over MgSO₄, filtered and concentrated under reduced pressure to give the desired product as a colorless solid (29.0 g, 123.8 mmol, 73% yield); LRMS m/z (APCI⁺) 235 [M+H]; 400 MHz ¹HNMR (CDCl₃) δ 7.31-7.24 (m, 3H); 7.20 (dd, J=5.0, 5.0 Hz, 1H); 6.51 (s, 1H); 5.58 (dd, J=2.9, 2.9 Hz, 1H); 4.64 (ddd, J=5.8, 4.2, 4.2 Hz, 1H); 2.58 (ddd, J=10.4, 9.1, 6.6 Hz, 2H); 2.55-2.48 (m, 1H); 2.39 (s, 3H); 2.27-2.18 (m, 1H); 100 MHz ¹³C NMR (CDCl₃) δ 170.5, 135.9, 135.4, 131.7, 129.2, 127.3, 126.5, 82.6, 55.5, 27.3, 21.8, 19.1.

Intermediate 53:

### Trans-5-Amino-6-o-tolyl-piperidin-2-one

Intermediate 52 (29.0 g, 123.9 mmol) was dissolved in 1.2 L of EtOH in a large 4 L parr bottle under N₂. Separately, 12.0 g of Raney Nickel was washed with H₂O (3×'s), followed by EtOH (2×'s). The catalyst was then added carefully to the reaction vessle. This solution was subjected to an H₂ atmosphere at 45 psi for 1½ h. At this time, another 12.0 g of fresh Raney Nickel was added, and the reaction resubjected to H₂ at 45 psi for an additional 1½ h. The solution was then filtered through a plug of celite and the filtrate was concentrated under reduced pressure to give a semi-solid material. Trituration with Et₂O gave the desired product as a colorless crystalline solid (22.7 g, 111.3 mmol, 90% yield); LRMS m/z (APCI⁺) 205 [M+H]; 500 MHz ¹HNMR (CDCl₃) δ 7.32 (dd, J=9.1, 1.7 Hz, 1H); 7.26-7.13 (m, 3H); 5.77 (s, 1H); 4.47 (d, J=7.9 Hz, 1H); 3.15 (ddd, J=10.4, 7.9, 3.3 Hz, 1H); 2.56 (ddd, J=10.0, 7.1, 4.2 Hz, 2H); 2.40 (s, 3H); 2.01 (dddd, J=6.9, 4.1, 4.1 4.1 Hz, 1H); 1.80 (dddd, J=17.0, 10.0, 10.0, 7.1 Hz, 1H); 125 MHz ¹³C NMR (CDCl₃) δ.171.9, 138.2, 136.5, 131.1, 128.4, 127.0, 61.1, 52.5, 29.7, 28.7, 19.8.

Intermediate 54:

### Trans-(6-Oxo-2-o-tolyl-piperidin-3-yl)-carbamic acid tert-butyl ester

Intermediate 53 (21.6 g, 106.0 mmol) was dissolved in 500 mL of anhydrous acetonitrile and 74.0 mL of Et₃N. Boc anhydride (23.1 g, 106.0 mmol) was added and the reaction was stirred at rt for 1 h. As the starting material was consumed, the product precipitated out of solution. The reaction was concentrated down to ½ volume and the solids were collected through suction filtration. Rinsing with 1:1 Et₂O/Hexanes gave the desired material cleanly (19.9 g, 65.4 mmol, 62% yield); LRMS m/z (APCI⁺) 305 [M+H]; 500 MHz ¹HNMR (CDCl₃) δ 7.52-7.15 (m, 4H); 5.89 (bs, 1H); 4.91 (bs, 1H); 4.73 (bs, 1H); 3.95 (bs, 1H); 2.56 (ddd, J=6.2, 6.2, 6.2 Hz, 2H); 2.43 (s, 3H); 2.04-1.96 (m, 1H); 1.84 (dddd, J=12.9, 6.2, 6.2, 6.2 Hz, 1H); 1.38 (s, 9H).

Intermediate 55:

### (2-o-Tolyl-piperidin-3-yl)-carbamic acid tert-butyl ester

Intermediate 54 (1.00 g, 3.28 mmol) was dissolved in 30 mL of anhydrous THF under N₂. A 1.0 M solution of BH₃ (6.56 mL) was added at rt over 10 min. The reaction was then heated in an oil bath at 85° C. for 18 h. The reaction was then cooled and diluted in CH₂Cl₂ and washed with aqueous saturated NaHCO₃. The organics were then dried over MgSO₄, filtered and concentrated under reduced pressure to give the crude product. Purification was accomplished through flash chromatography on a 35 g Isco silica gel column using a gradient eluent of 20-25% acetone/hexanes and collecting 8 mL fractions. Product containing fractions were combined and concentrated under reduced pressure to give the desired material as a colorless solid (0.43 g, 1.48 mmol, 45% yield); LRMS m/z (APCI⁺) 291 [M+H]; 500 MHz ¹HNMR (CD₃OD) δ 7.48 (dd, J=7.1, 0.0 Hz, 1H); 7.16-7.08 (m, 3H); 4.86 (s, 2H); 3.77-3.71 (m, 2H); 3.04 (dd, J=12.4, 0.0 Hz, 1H); 2.65 (ddd, J=12.0, 12.0, 0.0 Hz, 1H); 2.43 (s, 3H); 2.08 (dd, J=12.0, 0.0 Hz, 1H); 1.80-1.66 (m, 2H); 1.58-1.50 (m, 1H); 1.22 (s, 9H).

Intermediate 56:

### (1-{[1-(R)-(3,5-Bis-trifluoromethyl-phenyl)-ethyl]-methyl-carbamoyl}-2-o-tolyl-piperidin-3-yl)-carbamic acid tert-butyl ester (less polar)

Using intermediate 55 and benzylamine [1-(3,5-bis-trifluoromethyl-phenyl)-ethyl]-methylamine, the same procedure was followed as for intermediate 13. The desired product was obtained as a pale solid and the isomers separated through flash chromatography on a 35 g Isco silica gel column. The less polar isomer was isolated as a colorless solid (0.13 g, 0.22 mmol, 15% yield); Rf 0.5 (40% EtOAc/Hexanes); LRMS m/z (APCI⁺) 588 [M+H];

Intermediate 57:

### 3-Amino-2-o-tolyl-piperidine-1-carboxylic acid [1-(3,5-bis-trifluoromethyl-phenyl)-ethyl]-methyl-amide

Intermediate 56 (0.10 g, 0.17 mmol) was dissolved in 1.7 mL of DCE and 270 uL of Et₃Si (1.70 mmol) was added, followed by 270 uL of TFA (3.41 mmol). The reaction was heated in an oil bath at 75° C. for 4 hours and then cooled to rt overnight. The solution was then concentrated under reduced pressure to give the crude product. Purification was accomplished through flash chromatography using a 15 g Isco silica gel column and eluting with 5% MeOH/CH₂CL₂ with 0.2% NH₄OH and collecting 8 mL fractions. Product containing fractions were combined and concentrated under reduced pressure to give the desired material as a colorelss oil (0.70 g, 0.14 mmol, 85% yield); Rf 0.5 (10% MeOH/CH₂Cl₂ with 0.2% NH₄0H); LRMS m/z (APCI⁺) 488 [M+H].

Intermediate 58:

(1-{[1-(3,5-Bis-trifluoromethyl-phenyl)-ethyl]-methyl-carbamoyl}-2-o-tolyl-piperidin-3-yl)-methyl-carbamic acid tert-butyl ester

Intermediate 56 (0.81 g, 1.37 mmol) was dissolved in anhydrous THF and MeI (0.97 g, 6.87 mmol) was added, followed by dropwise addition of a 1.0 M solution of KOtBu in THF (6.87 mmol). The cloudy solution was stirred for 1 h at rt. The reaction was then diluted in CH₂Cl₂ and extracted with aqueous saturated NaHCO₃, dried over MgSO₄, filtered and concentrated to give the desired material as a pale solid (0.82 g, 1.37 mmol, 100% yield); LRMS m/z (APCI⁺) 602 [M+H.

Intermediate 59:

### 3-Methylamino-2-o-tolyl-piperidine-1-carboxylic acid [1-(3,5-bis-trifluoromethyl-phenyl)-ethyl]-methyl-amide

Using intermediate 58 and following the same procedure as used for intermediate 57 gave the desired product as a colorless solid (0.21 g, 0.41 mmol, 30% yield); Rf 0.3 (5% MeOH/CH₂Cl₂ with 0.2% NH₄OH; LRMS m/z (APCI⁺) 502 [M+H]; 500 MHz ¹HNMR (CD₃OD) δ 7.85 (s, 1H); 7.70 (s, 2H); 7.41 (dd, J=5.8 Hz, 1H); 7.17-7.11 (m, 3H); 5.43 (q, J=6.9 Hz, 1H); 4.46 (d, J=8.3 Hz, 1H); 3.34 (s, 3H); 3.32-3.29 (m, 1H); 3.20 (ddd, J=9.5, 9.5, 3.7 Hz, 1H); 2.98 (ddd, J=10.4, 10.4, 3.3 Hz, 1H); 2.69 (s, 3H); 2.43 (s, 3H); 2.38-2.31 (m, 1H); 1.97-1.82 (m, 2H); 1.62 (ddd, J=23.6, 10.4, 4.6 Hz, 1H); 1.47 (d, J=7.1 Hz, 3H).

Intermediate 60:

### 4-Methyl-N-(2-methyl-benzylidene)-benzenesulfonamide

O-tolualdehyde (50.0 g, 416.0 mmol) and p-toluenesulfonamide (74.7 g, 437.0 mmol) were combined in 500 mL of anyhydrous toluene. BF₃.OEt₂ (0.8 mL, 8.3 mmol) was added dropwise to the reaction and the resulting solution was heated in an oil bath at 105° C. equipped with a Dean Stark trap and reflux condensor. After 3½ h heating, an additional portion of BF₃.OEt₂ (0.8 mL, 8.3 mmol) was added and the reaction was heated for an additional 16 h. The solution was then cooled to rt and quenched with saturated aqueous NaHCO₃. The reaction was then extracted with EtOAc (3×400 mL) and the combined organics were dried over MgSO₄, filtered and concentrated. The crude material was then triturated in Et₂O overnight and the clean solids filtered and dried to obtain the desired product as colorless crystals (74.5 g, 273 mmol, 66% yield); LRMS m/z (APCI⁺) 274 [M+H]; 500 MHz ¹HNMR (CDCl₃) δ 9.33 (s, 1H); 7.89 (ddd, J=8.3, 2.1, 2.1 Hz, 1H); 7.81 (ddd, J=8.3, 2.1, 2.1 Hz, 2H); 7.35-7.25 (m, 5H); 2.43 (s, 3H); 2.42 (s, 3H).

Intermediate 61:

### 1-(Toluene-4-sulfonyl)-2-o-tolyl-2,5-dihydro-1H-pyrrole-3-carboxylic acid ethyl ester

Intermediate 60 (74.0 g, 271.0 mmol) was dissolved in 740 mL of anhydrous toluene and ethyl-2-butynoate (30.4 g, 271.0 mmol) was added, followed by tributylphosphine (5.5 g, 27.1 mmol). The resulting solution was then heated in an oil bath at 85° C. for 1 h. The reaction was cooled to rt and concentrated under reduced pressure to give a pale solid. This material was triturated with isopropyl ether and left stirring in solution for 2 days. The solids were then filtered and dried to give the clean product as a colorless crystalline solid (60.0 g, 156.0 mmol, 58% yield). Seperation of enatiomers was accomplished using a chiral column.

Intermediate 62:

### 1-(Toluene-4-sulfonyl)-2-(S)-o-tolyl-2,5-dihydro-1H-pyrrole-3-carboxylic acid ethyl ester (less polar)

colorless crystalline solid (7.2 g, 18.8 mmol): Chiralcel OD (10 cm×50 cm) 250 mL/min, 83/17 Heptane/EtOH, retention time 9.69 min; LRMS m/z (APCI⁺) 386 [M+H]; 500 MHz ¹HNMR (CDCl₃) δ 7.31 (d, J=8.3 Hz, 2H); 7.10-7.05 (m, 4H); 6.94-6.90 (m, 1H); 6.83-6.79 (m, 2H); 6.06 (ddd, J=4.2, 2.5, 2.5 Hz, 1H); 4.57 (ddd, J=17.0, 2.5, 2.5 Hz, 1H); 4.37 (ddd, J=17.0, 6.2, 2.1 Hz, 1H); 4.00 (dddd, J=25.3, 14.5, 10.8, 7.1 Hz, 2H); 2.55 (s, 3H); 2.34 (s, 3H); 1.08 (t, J=7.3 Hz, 3H); 125 MHz ¹³C NMR (CDCl₃) δ 162.0, 143.3, 137.8, 136.9, 136.6, 136.0, 135.4, 130.6, 129.6, 127.9, 127.7, 127.1, 126.2, 65.0, 61.1, 55.1, 21.7, 19.4, 14.1.

Intermediate 63:

### 1-(Toluene-4-sulfonyl)-2-(R)-o-tolyl-2,5-dihydro-1H-pyrrole-3-carboxylic acid ethyl ester (more polar)

colorless crystalline solid (8.7 g, 22.6 mmol): Chiralcel OD (10 cm×50 cm) 250 mL/min, 83/17 Heptane/EtOH, retention time 6.75 min; LRMS m/z (APCI⁺) 386 [M+H]; 500 MHz ¹HNMR (CDCl₃) δ 7.31 (d, J=8.3 Hz, 2H); 7.10-7.08 (m, 4H); 6.92 (ddd, J=7.9, 5.4, 3.3 Hz, 1H); 6.82 (d, J=7.5 Hz, 1H); 6.80 (ddd, J=2.1, 2.1, 2.1 Hz, 1H); 6.06 (ddd, J=4.2, 2.1, 2.1 Hz, 1H); 4.56 (ddd, J=16.6, 2.5, 2.5 Hz, 1H); 4.38 (ddd, J=17.0, 6.2, 2.1 Hz, 1H); 4.00 (dddd, J=25.3, 14.1, 10.8, 7.1 Hz, 2H); 2.55 (s, 3H); 2.34 (s, 3H); 1.08 (t, J=7.1 Hz, 3H); 100 MHz ¹³C NMR (CDCl₃) δ 162.0, 143.3, 137.8, 136.9, 136.6, 136.0, 135.4, 130.6, 129.6, 127.9, 127.7, 127.1, 126.2, 65.6, 61.1, 55.1, 21.7, 19.4, 14.1.

Intermediate 64:

### Cis-1-(Toluene-4-sulfonyl)-2-o-tolyl-pyrrolidine-3-carboxylic acid ethyl ester

Intermediate 62 (7.0 g, 18.2 mmol) was dissolved in 100 mL EtOH in a par bottle and flushed with N₂. Pd/C (0.7 g, 10% by weight) was then added and the reaction was subjected to an H₂ atmosphere at 45 psi for 16 h. The catalyst was then filtered off through a plug of celite and the filtrate concentrated to give the desired product as a pale solid (6.8 g, 17.6 mmol, 97% yield); LRMS m/z (APCI⁺) 388 [M+H]; 500 MHz ¹HNMR (CDCl₃) δ 7.67 (d, J=8.3 Hz, 2H); 7.38-7.35 (m, 1H); 7.28 (d, J=7.9 Hz, 2H); 7.17-7.12 (m, 3H); 5.29 (d, J=2.9 Hz, 1H); 3.92-3.75 (m, 3H); 3.51 (ddd, J=17.8, 9.1, 0.0 Hz, 1H); 2.75 (dddd, J=5.4, 2.9, 2.9, 2.9 Hz, 1H); 2.42 (s, 3H); 2.40 (s, 3H); 2.14-2.09 (m, 2H); 1.31 (t, J=7.3 Hz, 3H); 100 MHz ¹³C NMR (CDCl₃) δ 172.2, 143.6, 140.6, 135.1, 134.5, 130.8, 129.7, 127.9, 127.6, 126.4, 126.4, 63.3, 61.4, 52.1, 48.6, 26.6, 21.8, 19.6, 14.2.

Intermediate 65:

### 1-(Toluene-4-sulfonyl)-2-(R)-o-tolyl-pyrrolidine-3-(R)-carboxylic acid

Intermediate 64 (6.8 g, 17.6 mmol) was dissolved in a 1:1 solution of MeOH/1M NaOH (350 mL) and heated in an oil bath at 40° C. for 16 h. The reaction went from a clear, colorless solution to a cloudy suspension upon completion. The reaction was cooled and concentrated to ½ volume under reduced pressure. The solution was then acidified with 1M HCl (pH=4.0) and extracted with CH₂Cl₂ (3×75 mL). The combined organics were then washed with brine, dried over Na₂SO₄, filtered and concentrated under reduced pressure to give the desired product as a colorless solid (6.3 g, 17.5 mmol, 100% yield); LRMS m/z (APCI⁺) 360 [M+H]; 500 MHz ¹HNMR (CDCl₃) δ 7.64 (d, J=8.3 Hz, 2H); 7.34-7.32 (m, 1H); 7.22 (d, J=7.9 Hz, 2H); 7.18-7.13 (m, 3H); 5.34 (d, J=2.5 Hz, 1H); 3.77 (dddd, J=7.9, 7.9, 7.9, 3.3 Hz, 1H); 3.49 (ddd, J=9.5, 9.5, 7.1 Hz 1H); 2.82 (ddd, J=6.2, 2.9, 2.9 Hz, 1H); 2.41 (s, 3H); 2.35 (s, 3H); 2.23-2.17 (m, 1H); 2.15-2.09 (m, 1H).

Intermediate 66:

### [1-(Toluene-4-sulfonyl)-2-(R)-o-tolyl-pyrrolidin-3-(R)-yl]-carbamic acid tert-butyl ester

Intermediate 65 (5.0 g, 13.9 mmol) was dissolved in 100 mL of t-butanol and Et₃N (2.1 mL, 15.3 mmol) was added, followed by diphenylphosphorylazide (3.1 mL, 15.3 mmol). The reaction was then heated in an oil bath at 75° C. for 6½ h. Upon cooling, the reaction was concentrated under reduced pressure, redissolved in CH₂Cl₂ and then extracted with saturated aqueous NaHCO₃, followed by H₂O and then brine. The combined organics were then dried over Na₂SO₄, filtered and concentrated under reduced pressure to give the crude product. Purification was accomplished through flash chromatography on a 75S Biotage silica gel column using a gradient eluent of 30-50% EtOAc/Hexanes and collecting 25 mL fractions. Product containing fractions were combined and concentrated under reduced pressure to give the desired material as a colorless solid (2.3 g, 5.3 mmol, 39% yield); Rf 0.8 (5% MeOH/CH₂Cl₂); LRMS m/z (APCI⁺) 431 [M+H].

Intermediate 67:

### (2-(R)-o-Tolyl-pyrrolidin-3-(R)-yl)-carbamic acid tert-butyl ester

A 1 M solution of Na/Naphthalene in DME was made by combining naphthalene (6.0 g, 46.8 mmol) and sodium (0.8 g, 32.2 mmol) in 46.0 mL of DME and stirring this solution for 48 hours before use. In a separate flask, intermeidate 66 (2.3 g, 5.4 mmol) was dissolved in 25 mL of DME and cooled to −78° C. under N₂. The 1.0 M Na/Naphthalene solution was then added dropwise to the reaction solution until the dark blue coloring was maintained in the reaction solution. The reaction was stirred for an additional 10 minutes and then quenched with 2 mL H₂O and allowed to warm to rt. The resulting solution was then concentrated under reduced pressure and the residual oil was redissolved in 1M NaOH and extracted with CH₂Cl₂ (2×25 mL). Combined organics were then dried over Na₂SO₄, filtered and concentrated to give the crude product. Purification was accomplished through flash chromatography on a 35 g Isco silica gel column using a gradient of 5-10% MeOH/CH₂Cl₂ and collecting 8 mL fractions. Product containing fractions were combined and concentrated under reduced pressure to give the desired material as a colorless solid (0.9 g, 3.2 mmol, 57% yield); LRMS m/z (APCI⁺) 177/277 [M+H]; ]; 500 MHz ¹HNMR (CD₃OD) δ 7.38 (dd, J=7.5, 0.0 Hz, 1H); 7.19-7.17 (m, 1H); 7.14-7.10 (m, 2H); 4.17 (dd, J=6.6, 0.0 Hz, 1H); 4.07 (dddd, J=6.2, 6.2, 6.2, 6.2 Hz, 1H); 3.18 (dddd, J=10.8, 10.8, 7.9, 7.9 Hz, 1H); 3.05 (dddd, J=7.5, 7.5, 7.5, 7.5 Hz, 1H); 2.40 (s, 3H); 2.25-2.16 (m, 1H); 1.80-1.73 (m, 1H); 1.38 (s, 9H).

Intermediate 68 (Also Example 241):

### (1-(R)-{[1-(3,5-Bis-trifluoromethyl-phenyl)-ethyl]-methyl-carbamoyl}-2-(S)-o-tolyl-pyrrolidin-3-(R)-yl)-carbamic acid tert-butyl ester

The benzylamine, [1-(R)-(3,5-Bis-trifluoromethyl-phenyl)-ethyl]-methyl-amine (1.13 g, 4.17 mmol) was dissolved in 40 mL anhydrous DCE and 2.30 mL (16.68 mmol) of anhydrous Et₃N. Triphosgene (0.41 g, 1.38 mmol) was separately dissolved in DCE and added dropwise to the reaction mixture under N₂. The resulting solution was stirred for 1½ h at rt. Intermediate 67 (0.87 g, 3.13 mmol) was dissolved in fresh DCE and added to the reaction and the solution was heated to reflux in an oil bath at 55° C. for 19 h. The reaction was then cooled to rt and extracted with saturated aqueous NaHCO₃ (2×50 mL). Combined organics were dried over Na₂SO₄, filtered and concentrated under reduced pressure. Purification was accomplished by flash chromatography on a 35 g Isco silica gel column eluting with 25% EtOAc/Hexanes and collecting 18 mL fractions. Product containing fractions were collected and concentrated under reduced pressure to give a colorless solid (1.69 g, 2.95 mmol, 94% yield); Rf 0.4 (40% EtOAc/Hexanes); LRMS m/z (APCI⁺) 574 [M+H]; 500 MHz ¹HNMR (CDCl₃) δ 7.68 (s, 1H); 7.52 (s, 2H); 7.17-7.02 (m, 4H); 5.26 (q, J=6.8 Hz, 1H); 5.13 (d, J=6.2 Hz, 1H); 4.99 (bs, 1H); 4.01-3.74 (m, 3H); 2.49 (s, 3H); 2.28 (s, 3H); 2.14 (dddd, J=12.9, 12.9, 7.1, 7.1 Hz, 1H); 1.86 (m, 1H); 1.47 (d, J=7.1 Hz, 3H); 1.36 (bs, 9H).

Intermediate 69:

### (1-{[1-(R)-(3,5-Bis-trifluoromethyl-phenyl)-ethyl]-methyl-carbamoyl}-2-(S)-o-tolyl-pyrrolidin-3-(R)-yl)-methyl-carbamic acid tert-butyl ester

Using intermediate 68, the same procedure was followed as for intermediate 58. The product was obtained as a pale solid (0.13 g, 0.21 mmol, 98% yield); LRMS m/z (APCI⁺) 588 [M+H]; 500 MHz ¹HNMR (CDCl₃) δ 7.71 (s, 1H); 7.54 (s, 2H); 7.21-7.19 (m, 1H); 7.13-7.02 (m, 3H); 5.29 (q, J=6.3 Hz, 1H); 5.03 (d, J=7.9 Hz, 1H); 4.64 (ddd, J=11.2, 8.7, 6.2 Hz, 1H); 3.79-3.72 (m, 1H); 3.63-3.50 (m, 1H); 2.88 (bs, 3H); 2.50 (s, 3H); 2.30 (s, 3H); 2.21-2.16 (m, 1H); 2.06-2.03 (m, 1H); 1.53 (d, J=6.6 Hz, 3H); 1.22 (s, 9H).

Intermediate 70 (Also Example 242):

### 3-(R)-Amino-2-(S)-o-tolyl-pyrrolidine-1-(R)-carboxylic acid [1-(3,5-bis-trifluoromethyl-phenyl)-ethyl]-methyl-amide

Using intermediate 68, the same procedure was followed as for intermediate 59. The desired material was obtained as a colorless oil (15.00 mg, 0.03 mmol, 22% yield); Rf 0.3 (5% MeOH/CH₂Cl₂ with 0.2% NH₄OH); LRMS m/z (APCI⁺) 474 [M+H.

### Example 1 (Also Intermediate 12)

### 2-(4-Fluoro-2-methyl-phenyl)-3-hydroxymethyl-pyrrolidine-1-carboxylic acid [1-(3,5-bis-trifluoromethyl-phenyl)-ethyl]-methyl-amide

The racemic benzylamine, [1-(3,5-Bis-trifluoromethyl-phenyl)-ethyl]-methyl-amine (0.07 g, 0.22 mmol) was dissolved in 2 mL anhydrous DCE and 0.12 mL (0.85 mmol) of anhydrous Et₃N. Triphosgene (0.02 g, 0.07 mmol) was separately dissolved in DCE and added dropwise to the reaction mixture under N₂. The resulting solution was stirred for 1½ h at rt. Intermediate 11 (0.04 g, 0.21 mmol) was dissolved in fresh DCE and added to the reaction and the solution was then heated to reflux in an oil bath at 55° C. for 19 h. The reaction was then cooled to rt and extracted with saturated aqueous NaHCO₃ (2×10 mL). Combined organics were then dried over Na₂SO₄, filtered and concentrated under reduced pressure. Purification was accomplished by crystallization with hot isopropyl ether to give a colorless solid (0.10 g, 0.20 mmol, 95% yield): Rf 0.3 (50% EtOAc/Hexanes); LRMS m/z (APCI⁺) 507 [M+H]; 500 MHz ¹HNMR (CDCl₃) δ diagnostic peak of benzylic hydrogen of racemic side chain 5.42 (q, J=7.1 Hz, 1H) of trans isomer in a 1:1 ratio to the cis isomer; 5.31 (q, J=6.9 Hz, 1H).

### Example 2 (Also Intermediate 13)

### 2-(4-Fluoro-2-methyl-phenyl)-3-hydroxymethyl-pyrrolidine-1-carboxylic acid [1-(R)-(3,5-bis-trifluoromethyl-phenyl)-ethyl]-methyl-amide

The benzylamine, [1-(3,5-Bis-trifluoromethyl-phenyl)-ethyl]-methyl-amine (11.4 g, 42.1 mmol) was dissolved in 100 mL anhydrous DCE and 23.5 mL (168 mmol) of anhydrous Et₃N. Triphosgene (4.1 g, 13.9 mmol) was separately dissolved in DCE and added dropwise to the reaction mixture under N₂. The resulting solution was stirred for 1½ h at rt. Intermediate 11 (2.0 g, 9.6 mmol) was dissolved in fresh DCE and added to the reaction and the solution was then heated to reflux in an oil bath at 55° C. for 19 h. The reaction was then cooled to rt and extracted with saturated aqueous NaHCO₃ (2×50 mL). Combined organics were then dried over Na₂SO₄, filtered and concentrated under reduced pressure. Purification was accomplished by flash chromatography on a 75 S Biotage silica gel column eluting with 50% EtOAc/Hexanes and collecting 25 mL fractions. Product containing fractions were collected and concentrated under reduced pressure to give a colorless solid (17.4 g, 34.5 mmol, 82% yield); Rf 0.3 (50% EtOAc/Hexanes); LRMS m/z (APCI⁺) 507 [M+H].

Enantiomers Separated:

### Example 3 (Intermediate 14)

### 2-(S)-(4-Fluoro-2-methyl-phenyl)-3-(S)-hydroxymethyl-pyrrolidine-1-carboxylic acid [1-(R)-(3,5-bis-trifluoromethyl-phenyl)-ethyl]-methyl-amide

colorless crystalline solid: R, R Whelk O-1 (4.6 mm×25 cm) 1 mL/min, 85/15 Heptane/EtOH, retention time 9.08 min; [α]₂₂^{D}=+5.78° (c 1.00, CH₂Cl₂).

### Example 4 (Intermediate 15)

### 2-(R)-(4-Fluoro-2-methyl-phenyl)-3-(R)-hydroxymethyl-pyrrolidine-1-carboxylic acid [1-(R)-(3,5-bis-trifluoromethyl-phenyl)-ethyl]-methyl-amide

colorless crystalline solid: R, R Whelk O-1 (4.6 mm×25 cm) 1 mL/min, 85/15 Heptane/EtOH, retention time 11.09 min; [α]₂₂^{D}=+93.5° (c 1.02, CH₂Cl₂).

### Example 5 (Intermediate 21)

### 2-(S)-(4-Fluoro-2-methyl-phenyl)-3-(S)-hydroxymethyl-pyrrolidine-1-(S)-carboxylic acid [1-(3,5-bis-trifluoromethyl-phenyl)-ethyl]-methyl-amide

Intermediate 20 (3.0 g, 11.0 mmol) was dissolved in 50 mL anhydrous DCE and 6.13 mL (44.0 mmol) of anhydrous Et₃N. Triphosgene (1.1 g, 3.6 mmol) was separately dissolved in DCE and added dropwise to the reaction mixture under N₂. The resulting solution was stirred for 1½ h at rt. Intermediate 19 was dissolved in fresh DCE and added to the reaction and the solution was heated to reflux in an oil bath 55° C. for 19 h. The reaction was then cooled to rt and extracted with saturated aqueous NaHCO₃ (2×25 mL). Combined organics were dried over Na₂SO₄, filtered and concentrated under reduced pressure. Purification was accomplished by flash chromatography on a 75S Biotage silica gel column eluting with a gradient system of 25%, 50% EtOAc/Hexanes and collecting 25 mL fractions. Product containing fractions (8-90) were collected and concentrated under reduced pressure to give a colorless solid (17.4 g, 34.5 mmol, 82% yield); Rf 0.3 (50% EtOAc/Hexanes); LRMS m/z (APCI⁺) 507 [M+H].

### Example 6 (Intermediate 22)

### 2-(R)-(4-Fluoro-2-methyl-phenyl)-3-(R)-hydroxymethyl-pyrrolidine-1-(S)-carboxylic acid [1-(3,5-bis-trifluoromethyl-phenyl)-ethyl]-methyl-amide

Intermediate 20 (2.59 g, 9.56 mmol) was dissolved in 100 mL anhydrous DCE and 5.33 mL (38.20 mmol) of anhydrous Et₃N. Triphosgene (0.94 g, 3.15 mmol) was separately dissolved in DCE and added dropwise to the reaction mixture under N₂. The resulting solution was stirred for 1½ h at rt. Intermediate 18 was dissolved in fresh DCE and added to the reaction and the solution was then heated to reflux in an oil bath at 55° C. for 19 h. The reaction was then cooled to rt and extracted with saturated aqueous NaHCO₃ (2×25 mL). Combined organics were then dried over Na₂SO₄, filtered and concentrated under reduced pressure. Purification was accomplished by flash chromatography on a 40M Biotage silica gel column eluting with a gradient system of 5%, 10% MeOH/CH₂Cl₂ and collecting 18 mL fractions. Product containing fractions (16-30) were collected and concentrated under reduced pressure to give a colorless solid (4.48 g, 8.85 mmol, 93% yield); Rf 0.5 (10% MeOH/CH₂Cl₂); LRMS m/z (APCI⁺) 507 [M+H].

### Example 7

### 3-(S)-Aminomethyl-2-(R)-(4-fluoro-2-methyl-phenyl)-pyrrolidine-1-carboxylic acid [1-(R)-(3,5-bis-trifluoromethyl-phenyl)-ethyl]-methyl amide

Intermediate 24 (0.10 g, 0.20 mmol) was dissolved in 5 mL of 7N NH₃ in methanol and stirred at room temperature under N₂ for 2½ hours. Sodium borohydride (0.01 g, 0.20 mmol)) was then added, resulting in gas evolution. The reaction mixture was stirred at rt for an additional ½ h and then concentrated to give a yellow oil. The crude material was purified by flash chromatography on a 10 g Isco silica gel column eluting with a gradient of 5%, 10%, 20% MeOH/CH₂Cl₂ with 0.1% NH₄OH), collecting 8 mL fractions. The product containing fractions (36-50) were then concentrated under reduced pressure to give a clear, colorless oil (0.017 g, 0.03 mmol, 17% yield over two steps); Rf 0.35 (10% MeOH/CH₂Cl₂ with 0.1% NH₄OH); LRMS m/z (APCI⁺) 507 [M+H]; 500 MHz ¹HNMR (CDCl₃) δ 7.75 (s, 1H); 7.58 (s, 2H); 7.18 (dd, J=7.9, 5.8 Hz, 1H); 6.85-6.80 (m, 2H); 5.30 (q, J=6.6 Hz, 1H); 4.88 (d, J=8.3 Hz, 1H); 3.73 (ddd, J=14.9, 14.9, 8.7 Hz, 1H); 3.58 (ddd, J=9.1, 0.0, 0.0 Hz, 1H); 2.86-2.59 (m, 2H); 2.50 (s, 3H); 2.40 (s, 3H); 2.23-2.15 (m, 2H); 1.76 (dddd, J=19.5, 10.0, 10.0, 10.0 Hz, 1H); 1,54 (d, J=6.6 Hz, 3H).

### Example 8

### 3-(R)-Dimethylaminomethyl-2-(S)-(4-fluoro-2-methyl-phenyl)-pyrrolidine-1-carboxylic acid [1-(R)-(3,5-bis-trifluoromethyl-phenyl)-ethyl]-methyl-amide

Intermediate 27 (0.21 g, 0.42 mmol) was dissolved in 10 mL of anhydrous THF under N₂. Dimethylamine (2M solution in Methanol, 4.16 mL, 8.32 mmol) was added and the reaction mixture was stirred at rt for 12 hours. Sodium triacetoxyborohydride (0.18 g, 0.83 mmol) was then added and the resulting suspension was stirred for an additional hour. The reaction was quenched with saturated aqueous NaHCO₃ and extracted with EtOAc (3×20 mL). Combined organics were dried over Na₂SO₄, filtered and concentrated to give a brown oil. Purification was accomplished by flash chromatography on a 10 g Isco silica gel column eluting with a gradient of 2%, 5%, 10% MeOH/CH₂Cl₂, collecting 8 mL fractions. The product containing fractions (12-36) were concentrated under reduced pressure to give a pale tan oil (0.05 g, 0.09 mmol, 21% yield over two steps); Rf 0.5 (10% MeOH/CH₂Cl₂); LRMS m/z (APCI⁺) 534 [M+H]; 500 MHz ¹HNMR (CDCl₃) δ 7.70 (s, 1H); 7.59 (s, 2H); 7.18 (dd, J=8.3, 5.8 Hz, 1H); 6.86-6.79 (m, 2H); 5.41 (q, J=7.1 Hz, 1H); 4.86 (d, J=6.6 Hz, 1H); 3.69 (ddd, J=10.0, 10.0, 6.6 Hz, 1H); 3.64 (ddd, J=8.3, 8.3, 3.3 Hz, 1H); 2.54 (s, 3H); 2.41 (s, 3H), 2.41-2.21 (m, 4H); 2.18 (s, 6H); 1.74 (dddd, J=13.7, 10.4, 10.4, 10.4 Hz, 1H); 1.48 (d, J=7.1 Hz, 3H).

### Example 9

### 2-(R)-(4-Fluoro-2-methyl-phenyl)-3-(S)-methylaminomethyl-pyrrolidine-1-carboxylic acid [1-(S)-(3,5-bistrifluoromethyl-phenyl)-ethyl]-methyl-amide

Using intermediate 25, the same procedure was followed as for example 8. The product was obtained as a pale tan oil (24.2 mg, 16% yield over two steps); Rf 0.3 (10% MeOH/CH₂Cl₂); LRMS m/z (APCI⁺) 520 [M+H].

### Example 10

### 3-(S)-Dimethylaminomethyl-2-(R)-(4-fluoro-2-methyl-phenyl)-pyrrolidine-1-carboxylic acid [1-(S)-(3,50bix-trifluoromethyl-phenyl)-ethyl]-methyl-amide

Using intermediate 25, the same procedure was followed as for example 8. The product was obtained as a pale oil (74.5 mg, 47% yield over two steps); Rf 0.5 (10% MeOH/CH₂Cl₂); LRMS m/z (APCI⁺) 534 [M+H]; 500 MHz ¹HNMR (CDCl₃) δ 7.71 (s, 1H); 7.59 (s, 2H); 7.18 (dd, J=8.3, 5.8 Hz, 1H); 6.87-6.80 (m, 2H); 5.41 (q, J=6.6 Hz, 1H); 4.87 (d, J=6.6 Hz, 1H); 3.74-3.62 (m, 2H); 2.54 (s, 3H); 2.41 (s, 3H); 2.28-2.21 (m, 4H); 2.19 (s, 6H); 1.78-1.73 (m, 1H); 1.48 (d, J=7.1 Hz, 3H).

### Example 11

### 2-(S)-(4-Fluoro-2-methyl-phenyl)-3-(R)-[(isopropyl-methyl-amino)-methyl]-pyrrolidine-1-carboxylic acid [1-(S)-(3,5-bis-trifluoromethyl-phenyl)-ethyl]-methyl-amide

Using intermediate 26, the same procedure was followed as for example 8. The product was obtained as a clear, colorless oil (41.0 mg, 37% yield over two steps); Rf 0.4 (10% MeOH/CH₂Cl₂); LRMS m/z (APCI⁺) 562 [M+H]; 500 MHz ¹HNMR (CDCl₃) δ 7.72 (s, 1H); 7.54 (s, 2H); 7.15 (dd, J=8.7, 6.2 Hz, 1H); 6.84-6.75 (m, 2H); 5.25 (q, J=7.1 Hz, 1H); 4.85 (d, J=5.4 Hz, 1H); 3.68 (apt t, J=6.2 Hz, 2H); 2.77-2.76 (m, 1H); 2.48 (s, 3H); 2.33 (d, J=6.2 Hz, 2H); 2.28 (s, 3H); 2.20-2.07 (m, 2H); 2.10 (s, 3H); 1.68 (dddd, J=16.6, 16.6, 8.7, 0.0 Hz, 1H); 1.49 (d, J=6.6 Hz, 3H); 0.96 (d, J=6.6 Hz, 3H); 0.89 (d, J=6.6 Hz, 3H).

### Example 12

### 3-(R)-(4-Ethyl-piperazin-1-ylmethyl)-2-(S)-(4-fluoro-2-methyl-phenyl)-pyrrolidine-1-carboxylic acid [1-(S)-(3,5-bis-trifluoromethyl-phenyl)-ethyl]-methyl-amide

Using intermediate 26, the same procedure was followed as for example 8. The product was obtained as a clear, colorless oil (42.9 mg, 36% yield over two steps); Rf 0.4 (10% MeOH/CH₂Cl₂); LRMS m/z (APCI⁺) 603 [M+H]; 500 MHz ¹HNMR (CDCl₃) δ 7.71 (s, 1H); 7.53 (s, 2H); 7.12 (dd, J=8.3, 5.8 Hz, 1H); 6.81-6.75 (m, 2H); 5.25 (q, J=7.1 Hz, 1H); 4.90 (d, J=6.6 Hz, 1H); 3.70 (ddd, J=9.6, 9.6, 9.6 Hz, 1H); 3.62-3.55 (m, 1H); 2.46 (s, 3H); 2.44-2.25 (m, 16H); 2.11-2.04 (m, 1H); 1.68 (dddd, J=12.5, 8.3, 8.3, 8.3 Hz, 1H); 1.50 (d, J=7.1 Hz, 3H); 1.05 (t, J=7.1 Hz, 3H).

### Example 13

### 3-(R)-Azetidin-1-ylmethyl-2-(S)-(4-fluoro-2-metyl-phenyl)-pyrrolidine-1-carboxylic acid [1-(S)-(3,5-bistrifluoromethyl-phenyl)-ethyl]-methyl-amide

Using intermediate 26, the same procedure was followed as for example 8. The product was obtained as a tan oil (20.0 mg, 19% yield over two steps); Rf 0.45 (10% MeOH/CH₂Cl₂); LRMS m/z (APCI⁺) 546 [M+H]; 500 MHz ¹HNMR (CDCl₃) δ 7.74 (s, 1H); 7.56 (s, 2H); 7.17 (dd, J=6.2, 6.2 Hz, 1H); 6.85-6.81 (m, 2H); 5.29 (q, J=6.6 Hz, 1H); 4.82 (d, J=9.1 Hz, 1H); 3.72 (ddd, J=10.0, 10.0, 6.2 Hz, 1H); 3.55 (ddd, J=10.3, 2.1, 2.1 Hz, 1H); 3.32 (apt q, J=7.1 Hz, 4H); 2.60-2.49 (m, 2H); 2.49 (s, 3H); 2.41 (s, 3H); 2.30-2.25 (m, 1H); 2.16-2.09 (m, 3H); 1.74 (dddd, J=10.0, 10.0, 10.0, 10.0 Hz, 1H); 1.53 (d, J=7.1 Hz, 3H).

### Example 14

### 3-(R)-Cyclopropylaminomethyl-2-(S)-(4-fluoro-2-methyl-phenyl)-pyrrolidine-1-carboxylic acid [1-(S)-(3,5-bis-trifluoromethyl-phenyl)-ethyl]-methyl-amide

Using intermediate 26, the same procedure was followed as for example 8. The product was obtained as a clear oil (23.3 mg, 22% yield over two steps); Rf 0.5 (10% MeOH/CH₂Cl₂); LRMS m/z (APCI⁺) 546 [M+H]; 500 MHz ¹HNMR (CDCl₃) δ 7.74 (s, 1H); 7.57 (s, 2H); 7.17 (dd, J=7.9, 5.8 Hz, 1H); 6.85-6.80 (m, 2H); 5.29 (q, J=6.6 Hz, 1H); 4.87 (d, J=7.9 Hz, 1H); 3.72 (ddd, J=9.5, 9.5, 6.6 Hz, 1H); 3.58 (ddd, J=9.5, 9.5, 6.6 Hz, 1H), 2.80-2.69 (m, 2H); 2.49 (s, 3H); 2.40 (s, 3H); 2.25-2.14 (m, 2H); 2.06 (dddd, J=6.6, 6.6, 3.3, 3.3 Hz, 1H); 1.74 (dddd, J=6.6, 6.6, 3.3, 3.3, 1H), 1.53 (d, J=7.1 Hz, 3H); 0.43-0.39 (m, 2H); 0.31 (d, J=2.9 Hz, 2H).

### Example 15

### 2-(S)-(4-Fluoro-2-methyl-phenyl)-3-(R)-{[methyl-piperidin-4-yl)-amino]-methyl}-pyrrolidine-1-carboxylic acid [1-(S)-(3,5-bis-trifluoromethyl-phenyl)-ethyl]-methyl-amide

Using intermediate 26, the same procedure was followed as for example 8. The product was obtained as a clear oil (31.1 mg, 25% yield over two steps); Rf 0.2 (10% MeOH/CH₂Cl₂); LRMS m/z (APCI⁺) 617 [M+H].

### Example 16

### 2-(S)-(4-Fluoro-2-methyl-phenyl)-3-(R)-(4-methyl-pierazin-1-ylmethyl)-pyrrolidine-1-carboxylic acid [1-(S)-(3,5-bis-trifluoromethyl-phenyl)-ethyl]-methyl-amide

Using intermediate 26, the same procedure was followed as for example 8. The product was obtained as a pale oil (42.0 mg, 36% yield over two steps); Rf 0.35 (10% MeOH/CH₂Cl₂); LRMS m/z (APCI⁺) 589 [M+H]; 500 MHz ¹HNMR (CDCl₃) δ 7.72 (s, 1H); 7.54 (s, 2H); 7.13 (dd, J=8.3, 5.8 Hz, 1H); 6.83-6.76 (m, 2H); 5.26 (q, J=7.0 Hz, 1H); 4.91 (d, J=7.1 Hz, 1H); 3.75-3.68 (m, 1H) 3.61 (ddd, J=8.3, 8.3, 3.7 Hz, 1H); 2.47 (s, 3H); 2.40 (s, 3H); 2.39-2.22 (m, 11H); 2.27 (s, 3H); 2.08 (dddd, J=10.0, 10.0, 10.0, 6.2 Hz, 1H); 1.69 (dddd, J=12.4, 8.3, 8.3, 8.3 Hz, 1H); 1.51 (d, J=6.6 Hz, 3H).

### Example 17

### 3-(R)-(3-Dimethylamino-pyrrolidin-1-ylmethyl)-2-(S)-(4-fluoro-2-methyl-phenyl)-pyrrolidine-1-carboxylic acid [1-(S)-(3,5-bis-trifluoromethyl-phenyl)-ethyl]-methyl-amide

Using intermediate 26, the same procedure was followed as for example 8. The product was obtained as a yellow oil (49.6 mg, 42% yield over two steps); Rf 0.25 (10% MeOH/CH₂Cl₂); LRMS m/z (APCI⁺) 603 [M+H]; 500 MHz ¹HNMR (CDCl₃) δ 7.73 (s, 1H); 7.56 (s, 2H); 7.15 (dd, J=8.3, 5.8 Hz, 1H); 6.83-6.77 (m, 2H); 5.28 (q, J=7.1 Hz, 1H); 4.86 (d, J=7.9 Hz, 1H); 3.73 (ddd, J=9.5, 9.5, 7.1 Hz, 1H); 3.59 (ddd, J=8.3, 8.3, 2.9 Hz, 1H); 2.68-2.56 (m, 2H); 2.48 (s, 3H); 2.42-2.10 (m, 10H); 2.39 (s, 3H); 2.18 (s, 3H); 1.89 (dddd, J=13.3, 8.3, 8.3, 8.3 Hz, 1H); 1.75 (ddd, J=11.6, 78.7, 8.7, 8.7 Hz, 1H); 1.66-1.62 (m, 1H); 1.53 (d, J=7.1 Hz, 3H).

### Example 18

### 2-(R)-(4-Fluoro-2-mehtyl-pheynyl)-3-(S)-piperidin-1-ylmethyl-pyrrolidine-1-carboxylic acid [1-(R)-(3,5-bis-trifluoromethyl-phenyl)-ethyl]-methyl-amide

Using intermedaite 24, the same procedure was followed as for example 8. The product was obtained as a pale yellow oil; Rf 0.3 (10% MeOH/CH₂Cl₂); LRMS m/z (APCI⁺) 574 [M+H].

### Example 19

### 3-(S)-Dimethylaminomethyl-2-(R)-(4-fluoro-2-methyl-phenyl)-pyrrolidine-1-carboxylic acid [1(R)-(3,5-bis-trifluoromethyl-phenyl)-ethyl]-methyl-amide

Using intermediate 24, the same procedure was followed as for example 8. The product was obtained as a pale oil (0.118 g, 57% yield over two steps); Rf 0.5 (10% MeOH/CH₂Cl₂); LRMS m/z (APCI⁺) 534 [M+H]; 500 MHz, ¹HNMR (CDCl₃) δ 7.73 (s, 1H); 7.55 (s, 2H); 7.17 (dd, J=8.3, 5.8 Hz, 1H); 6.84-6.76 (m, 2H); 5.26 (q, J=6.6 Hz, 1H); 4.86 (d, J=6.2 Hz, 1H); 3.73 (ddd, J=17.0, 10.0, 10.0, 10.0 Hz, 1H); 3.65 (ddd, J=8.7 8.7 3.7 Hz, 1H); 2.50 (s, 3H); 2.33 (s, 3H); 2.43-2.14 (m, 4H); 2.14 (s, 6H); 1.73 (dddd, J=12.0, 8.3, 8.3, 8.3 Hz, 1H); 1.52 (d, J=7.1 Hz, 3H).

### Example 20

### 2-(S)-(4-Fluror-2-methyl-phenyl)-3-(R)-methylaminomethyl-pyrrolidine-1-carboxylic acid [1-(R)-(3,5-bis-trufluoromethyl-phenyl)-ethyl]-methyl-amide

Using intermediate 27, the same procedure was followed as for example 8. The product was obtained as a pale tan oil (23.4 mg, 10% yield over two steps); Rf 0.2 (10% MeOH/CH₂Cl₂); LRMS m/z (APCI⁺) 520 [M+H]; 500 MHz ¹HNMR (CDCl₃) δ 7.70 (s, 1H); 7.57 (s, 2H); 7.20 (dd, J=8.7, 5.8 Hz, 1H); 6.88-6.81 (m, 2H); 5.42 (q, J=7.1 Hz, 1H); 4.87 (d, J=8.7 Hz, 1H); 3.80 (bs, 1H), 3.72 (ddd, J=10.0, 10.0, 6.2 Hz, 1H); 3.58 (ddd, J=14.5, 14.5, 0.0 Hz, 1H); 2.74-2.62 (m, 2H); 2.58 (s, 3H); 2.42 (s, 6H); 2.33-2.25 (m, 2H); 1.79 (dddd, J=19.5, 9.5, 9.5, 9.5 Hz, 1H); 1.48 (d, J=7.1 Hz, 3H).

### Example 21

### 2-(R)-(4-Fluoro-2-methyl-phenyl)-3-(S)-methylaminomethyl-pyrrolidine-1-carboxylic acid [1-(R)-(3,5-bis-trifluoromethyl-phenyl)ethyl]-methyl-amide

Using intermediate 24, the same procedure was followed as for example 8. The product was obtained as a clear, colorless oil (0.103 g, 51% yield over two steps); Rf 0.30 (10% MeOH/CH₂Cl₂); LRMS m/z (APCI⁺) 520 [M+H]; 500 MHz ¹HNMR (CDCl₃) δ 7.74 (s, 1H); 7.57 (s, 2H); 7.18 (dd, J=8.3, 5.8 Hz, 1H); 6.81 (d, J=5.8 Hz, 1H); 6.80 (d, J=9.9 Hz, 1H); 5.29 (q, J=6.6 Hz, 1H); 4.88 (d, J=8.3 Hz, 1H); 3.74 (ddd, J=10.0, 10.0, 6.6 Hz, 1H); 3.58 (ddd, J=7.9, 0.0, 0.0 Hz, 1H); 2.86 (bs, 1H); 2.68-2.58 (m, 2H); 2.50 (s, 3H), 2.40 (s, 3H), 2.38 (s, 3H), 2.29-2.18 (m, 2H); 1.76 (dddd, J=10.0, 10.0, 10.0, 10.0 Hz, 1H); 1.53 (d, J=7.0 Hz, 3H).

### Experimental Procedure for Examples 22-35 (Table 1)

The compounds listed in Table 1 were prepared of a mixture of isomers from Intermediate 23. The aldehyde (0.10 g, 0.20 mmol) was dissolved in 1 mL of anhydrous THF. Amine corresponding to sidechain “Z” in the scheme hereinbelow was then added and the reaction stirred at rt for 19 h. Na(OAc)₃BH (0.40 mmol) was added and the reaction was stirred for an additional hour. The reaction was then quenched with saturated aqueous NaHCO₃ extracted with EtOAc and the organics were dried and concentrated to give a crude brown oil. Purification was accomplished by flash chromatography on a 10 g Isco silca gel column.

TABLE 1

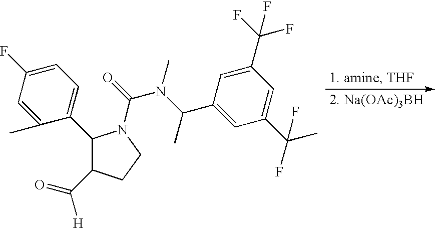

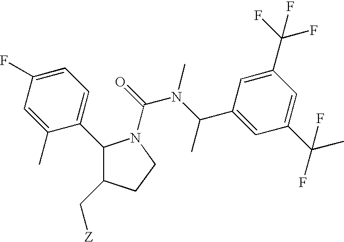

| Example | Z | m/z [+ H] |
|---|---|---|
| 22 | 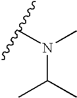 | 562 |
| 23 | 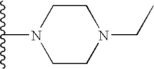 | 603 |
| 24 | 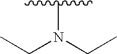 | 562 |
| 25 | 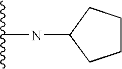 | 574 |
| 26 | 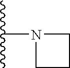 | 546 |
| 27 | 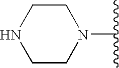 | 575 |
| 28 | 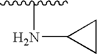 | 546 |
| 29 | 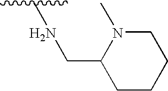 | 617 |
| 30 | 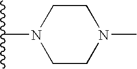 | 589 |
| 31 | 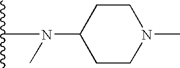 | 617 |
| 32 | 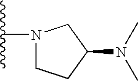 | 603 |
| 33 | 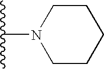 | 574 |
| 34 | 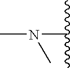 | 534 |
| 35 | 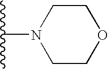 | 576 |

### Procedure for Examples 36-53

The compounds 36-53 were prepared using Intermediate 24. The amines (0.2 mmol, 4.0 eq) were pre-weighed in 1 dram, septa-capped vials. The aldehyde (0.05 mmol, 1.0 eq) was dissolved in 1 mL of anhydrous THF and added to the reaction vials and the resulting solutions shaken at rt for 16 h. To each reaction vial was then added Na(OAc)₃BH (30.00 mg, 2.5 eq) and the vials were shaken and addtitional 4.5 h. The reactions were quenched by adding 1N NaOH and 2.25 mL EtOAc. The organics were separated and loaded onto an equilibrated SCX SPE (conditioned with 5 mL MeOH, 2×5 mL EtOAc) columns. The desired products were then eluted off using 1N TEA in MeOH (5 mL). These solutions were collected in tared vials and dried under a N₂ stream. Purifications were accomplished by HPLC separation on a Waters Symmetry C¹⁸ column (5 mm, 3.9×150 mm) with a 1.0 mL/min flow rate eluting with a gradient system of 100%, 80%, 0% (0.1% TFA in H₂O/CH₃CN) injecting each sample in 2 mL of solvent.

### Example 36

### 2-(R)-(4-Fluoro-2-methyl-phenyl)-3-(S)-pyrrolidin-1-ylmethyl-pyrrolidine-1-carboxylic acid [1-(R)-(3,5-bis-trifluoromethyl-phenyl)-ethyl]-methyl-amide

LRMS m/z (APCI⁺) 560 [M+H]; 500 MHz ¹HNMR (CD₃OD) δ 7.88 (s, 1H); 7.70 (s, 2H); 7.44 (dd, J=7.8, 5.2 Hz, 1H); 6.94-6.90 (m, 2H); 5.29 (q, J=7.1 Hz, 1H); 4.93 (d, 1H); 4.00 (ddd, J=10.4, 10.4, 5.7 Hz, 1H); 3.71-3.64 (m, 3H); 3.51 (apt t, J=11.9 Hz, 1H); 3.14-3.09 (m, 2H); 2.85 (dddd, J=8.8, 8.8, 8.8, 0.0 Hz, 1H); 2.61-2.58 (m, 1H); 2.52 (s, 3H); 2.49 (s, 3H), 2.41 (dddd, J=5.7, 5.7, 5.7, 5.7 Hz, 1H); 2.12-2.07 (m, 2H); 2.03-1.91 (m, 3H); 1.66 (d, J=6.7, 3H).

### Example 37

### 2-(R)-(4-Fluoro-2-methyl-phenyl)-3-(S)-(4-methyl-piperidin-1-ylmethyl)-pyrrolidine-1-carboxylic acid [1-(R)-(3,5-bis-trifluoromethyl-phenyl)-ethyl]-methyl-amide

LRMS m/z (APCI⁺) 588 [M+H]; 500 MHz ¹HNMR (CD₃OD) δ 7.88 (s, 1H); 7.69 (s, 2H); 7.44 (dd, J=7.8, 5.7 Hz, 1H); 6.93-6.90 (m, 2H); 5.29 (q, J=6.9 Hz, 1H); 4.90 (s, 1H); 3.99 (ddd, J=9.9, 9.9, 5.7 Hz, 1H); 3.69 (ddd, J=8.8, 8.8, 0.0 Hz, 1H); 3.52 (ddd, J=11.9, 10.4, 0.0 Hz, 2H); 3.39 (ddd, J=13.0, 10.9, 0.0 Hz, 1H); 3.02-2.99 (m, 2H); 2.75 (dddd, J=13.0, 13.0, 3.1, 0.0 Hz, 1H); 2.80-2.62 (m, 1H), 2.52 (s, 3H); 2.50 (s, 3H); 2.45-2.41 (m, 1H); 1.93-1.86 (m, 3H); 1.65 (d, J=6.7 Hz, 3H); 1.67-1.62 (m, 1H), 1.42-1.40 (m, 2H); 1.00 (d, J=6.7 Hz, 3H).

### Example 38

### 2-(R)-(4-Fluoro-2-methyl-phenyl)-3-(S)-(isopropylamino-methyl)-pyrrolidine-1-carboxylic acid [1-(R)-(3,5-bis-trifluoromethyl-phenyl)-ethyl]-methyl-amide

LRMS m/z (APCI⁺) 548 [M+H]; 500 MHz ¹HNMR (CD₃OD) δ 7.88 (s, 1H); 7.70 (s, 2H); 7.42 (dd, J=8.3, 6.2 Hz, 1H); 6.94-6.90 (m, 2H); 5.30 (q, J=6.9 Hz, 1H); 4.95 (d, J=6.9 Hz, 1H); 3.99 (ddd, J=10.4, 10.4, 6.2 Hz, 1H); 3.71 (dddd, J=8.8, 8.8, 0.0 Hz, 1H); 3.36 (dddd, J=11.4, 6.2, 6.2, 6.2 Hz, 1H); 3.25 (dddd, J=11.9, 11.9, 0.0, 0.0 Hz, 1H); 2.93 (ddd, J=12.4, 2.6, 0.0 Hz, 1H); 2.53-2.45 (m, 1H); 2.53 (s, 3H); 2.49 (s, 3H); 2.39 (dddd, J=5.7, 5.7, 5.7, 5.7 Hz, 1H); 1.88 (dddd, J=11.4, 11.4, 11.4, 8.3 Hz, 1H); 1.66 (d, J=6.7 Hz, 3H); 1.29 (d, J=6.2 Hz, 6H).

### Example 39

### 2-(R)-(4-Fluoro-2-methyl-phenyl)-3-(S)-piperazin-1-ylmethyl-pyrrolidine-1-carboxylic acid [1-(R)-(3,5-bis-trifluoromethyl-phenyl)-ethyl]-methyl-amide

LRMS m/z (APCI⁺) 575 [M+H]; 500 MHz ¹HNMR (CD₃OD) δ 7.86 (s, 1H); 7.67 (s, 2H); 7.37 (dd, J=7.8, 5.7 Hz, 1H); 6.89-6.86 (m, 2H); 5.26 (q, J=7.1 Hz, 1H); 4.94 (d, J=8.8 Hz, 1H); 3.95 (ddd, J=9.9, 9.9, 6.2 Hz, 1H), 3.66 (ddd, J=1.6, 1.6, 1.6 Hz, 1H); 3.12-3.08 (m, 2H); 2.96 (bs, 2H); 2.72-2.48 (m, 7H); 2.51 (s, 3H); 2.50 (s, 3H); 2.21 (dddd, J=9.9, 5.7, 5.7, 5.7 Hz, 1H); 1.78 (dddd, J=18.7, 18.7, 10.4, 10.4 Hz, 1H); 1.65 (d, J=7.3 Hz, 3H).

### Example 40

### 2-(R)-(4-Fluoro-2-methyl-phenyl)-3-(S)-[(3-methylamino-propylamino)-methyl]-pyrrolidine-1-carboxylic acid [1-(R)-(3,5-bis-trifluoromethyl-phenyl)-ethyl]-methyl-amide

LRMS m/z (APCI⁺) 577 [M+H]; 500 MHz ¹HNMR (CD₃OD) δ 7.88 (s, 1H); 7.70 (s, 2H); 7.41 (dd, J=7.8, 5.7 Hz, 1H); 6.94-6.90 (m, 2H); 5.30 (q, J=6.9, Hz, 1H); 4.94 (d, 1H); 3.95 (ddd, J=10.4, 10.4, 6.2 Hz, 1H); 3.70 (ddd, J=8.8, 8.8, 0.0 Hz, 1H); 3.23 (dddd, J=11.9, 11.9, 0.0, 0.0 Hz, 1H); 3.21-2.99 (m, 5H); 2.71 (s, 3H); 2.60-2.50 (m, 1H); 2.52 (s, 3H); 2.48 (s, 3H); 2.44-2.38 (m, 1H); 2.14-2.02 (m, 2H); 1.09 (dddd, J=10.9, 10.9, 10.9, 8.3 Hz, 1H); 1.66 (d, J=7.3 Hz, 3H).

### Example 41

### 3-(S)-Azetidin-1-ylmethyl-2-(R)-(4-fluoro-2-methyl-phenyl)-pyrrolidine-1-carboxylic acid [1-(R)-(3,5-bis-trifluoromethyl-phenyl)-ethyl]-methyl-amide

LRMS m/z (APCI⁺) 546 [M+H]; 500 MHz ¹HNMR (CD₃OD) δ 7.88 (s, 1H); 7.69 (s, 2H); 7.42 (dd, J=8.8, 6.2 Hz, 1H); 6.94-6.90 (m, 2H); 5.29 (q, J=6.9 Hz, 1H); 4.90 (d, 1H); 4.27 (bs, 1H); 4.15 (m, 2H); 3.98-3.91 (m, 2H); 3.67 (ddd, J=8.8, 8.8, 0.0 Hz, 1H); 3.40-3.36 (m, 1H); 3.21 (ddd, J=12.4, 3.1, 0.0 Hz, 1H); 2.66-2.54 (m, 1H); 2.51 (s, 3H); 2.49 (s, 3H); 2.41-2.34 (m, 2H); 2.26 (ddd, J=11.4, 5.2, 5.2 Hz, 1H); 1.87 (dddd, J=11.4, 11.4, 11.4, 8.3 Hz, 1H); 1.65 (d, J=7.3 Hz, 3H).

### Example 42

### 3-(S)-[(Ethyl-methyl-amino)-methyl]-2-(R)-(4-fluoro-2-methyl-phenyl)-pyrrolidine-1-carboxylic acid [1-(R)-(3,5-bis-trifluoromethyl-phenyl)-ethyl]-methyl-amide

LRMS m/z (APCI⁺) 548 [M+H].

### Example 43

### 2-(R)-(4-Fluoro-2-methyl-phenyl)-3-(S)-(3-oxo-piperazin-1-ylmethyl)-pyrrolidine-1-carboxylic acid [1-(R)-(3,5-bis-trifluoromethyl-phenyl)-ethyl]-methyl-amide

LRMS m/z (APCI⁺) 589 [M+H]; 500 MHz ¹HNMR (CD₃OD) δ 7.88 (s, 1H); 7.69 (s, 2H); 7.42 (dd, J=9.3, 6.2 Hz, 1H); 6.93-6.89 (m, 2H); 5.28 (q, J=6.9 Hz, 1H); 4.94 (d, J=9.3 Hz, 1H); 3.98 (ddd, J=9.9, 9.9, 5.7 Hz, 1H); 3.69 (dd, J=8.8, 0.0 Hz, 2H); 3.53 (dd, J=16.1, 0.0 Hz, 1H); 3.45-3.38 (m, 2H); 3.30-3.20 (m, 3H); 2.99 (ddd, J=13.0, 4.2, 0.0 Hz, 1H); 2.67-2.63 (m, 1H); 2.51 (s, 3H); 2.49 (s, 3H); 2.36 (ddd, J=10.9, 5.2, 5.2 Hz, 1H); 1.88 (dddd, J=19.2, 10.9, 10.9, 10.9 Hz, 1H); 1.65 (d, J=7.3 Hz, 3H).

### Example 44

### 2-(R)-(4-Fluoro-2-methyl-phenyl)-3-(S)-[(2-morpholin-4-yl-ethylamino)-methyl]-pyrrolidine-1-carboxylic acid [1-(R)-(3,5-bis-trifluoromethyl-phenyl)-ethyl]-methyl-amide

LRMS m/z (APCI⁺) 619 [M+H]; 500 MHz ¹HNMR (CD₃OD) δ 7.88 (s, 1H); 7.70 (s, 2H); 7.43 (dd, J=7.8, 5.2 Hz, 1H); 6.93-6.89 (m, 2H); 5.30 (q, J=6.9 Hz, 1H); 4.94 (s, 1H); 3.99 (ddd, J=10.4, 10.4, 5.7 Hz, 1H); 3.83 (s, 4H), 3.71 (ddd, J=8.8, 8.8, 0.0 Hz, 1H); 3.43-3.00 (m, 10H), (2.59-2.56 (m, 1H); 2.52 (s, 3H); 2.49 (s, 3H); 2.42 (ddd, J=11.4, 5.2, 5.2 Hz, 1H); 1.90 (dddd, J=11.4, 11.4, 11.4, 8.3 Hz, 1H); 1.66 (d, J=7.3 Hz, 3H).

### Example 45

### 2-(R)-(4-Fluoro-2-methyl-phenyl)-3-(S)-[(2,2,2-trifluoro-ethylamino)-methyl]-pyrrolidine-1-carboxylic acid [1-(R)-(3,5-bis-trifluoromethyl-phenyl)-ethyl]-methyl-amide

LRMS m/z (APCI⁺) 588 [M+H]; 500 MHz ¹HNMR (CD₃OD) δ 7.87 (s, 1H); 7.69 (s, 2H); 7.38 (dd, J=9.3, 6.2 Hz, 1H); 6.92-6.89 (m, 2H); 5.28 (q, J=6.9 Hz, 1H); 4.95 (d, J=8.8 Hz, 1H); 3.95 (ddd, J=9.9, 9.9, 6.2 Hz, 1H); 3.70 (ddd, J=10.4, 2.1, 2.1 Hz, 1H); 3.63 (ddd, J=8.8, 8.8, 0.0 Hz, 2H); 3.07 (dddd, J=11.9, 11.9, 0.0, 0.0 Hz, 1H); 2.97 (ddd, J=11.9, 4.2, 0.0 Hz, 1H); 2.52 (s, 3H); 2.45 (s, 3H); 2.42-2.41 (m, 1H); 2.33 (ddd, J=10.4, 6.2, 4.2 Hz, 1H); 1.85 (dddd, J=18.7, 18.7, 10.4, 10.4 Hz, 1H); 1.65 (d, J=6.7 Hz, 3H).

### Example 46

### 3-(S)-[(2-Dimethylamino-ethylamino)-methyl]-2-(R)-(4-fluoro-2-methyl-phenyl)-pyrrolidine-1-carboxylic acid [1-(R)-(3,5-bis-trifluoromethyl-phenyl)-ethyl]-methyl-amide

LRMS m/z (APCI⁺) 577 [M+H]; 500 MHz ¹HNMR (CD₃OD) δ 7.88 (s, 1H); 7.70 (s, 2H); 7.42 (dd, J=8.3, 5.7 Hz, 1H); 6.93-6.91 (m, 2H); 5.30 (q, J=6.9 Hz, 1H); 4.94 (d, J=9.9 Hz, 1H); 3.99 (ddd, J=10.4, 10.4, 5.7 Hz, 1H); 3.65 (apt t, J=8.8 Hz, 1H), 3.48-3.46 (m, 4H); 3.28 (dddd, J=11.9, 11.9, 0.0, 0.0 Hz, 1H); 3.08 (ddd, J=11.9, 3.1, 0.0 Hz, 1H); 2.94 (s, 6H); 2.62-2.54 (m, 1H); 2.52 (s, 3H); 2.49 (s, 3H); 2.46-2.38 (m, 1H); 1.90 (dddd, J=11.4, 11.4, 11.4, 8.3 Hz, 1H); 1.66 (d, J=6.7 Hz, 3H).

### Example 47

### 2-(R)-(4-Fluoro-2-methyl-phenyl)-3-(S)-[(3-methoxy-propylamino)-methyl]-pyrrolidine-1-carboxylic acid [1-(R)-(3,5-bis-trifluoromethyl-phenyl)-ethyl]-methyl-amide

LRMS m/z (APCI⁺) 578 [M+H]; 500 MHz ¹HNMR (CD₃OD) δ 7.87 (s, 1H); 7.70 (s, 2H); 7.41 (dd, J=7.8, 7.8 Hz, 1H); 6.93-6.90 (m, 2H); 5.30 (q, J=6.9 Hz, 1H); 4.93 (d, J=9.9 Hz, 1H); 3.99 (ddd, J=10.4, 10.4 6.2 Hz, 1H); 3.70 (ddd, J=8.8, 8.8, 0.0 Hz, 1H); 3.49 (t, J=5.5 Hz, 2H); 3.32 (s, 3H); 3.20 (ddd, J=11.9, 11.9, 0.0 Hz, 1H); 3.11 (t, J=7.0 Hz, 2H); 3.01 (ddd, J=13.0, 3.1, 0.0 Hz, 1H); 2.52 (s, 3H); 2.52-2.48 (m, 1H); 2.48 (s, 3H); 2.38 (ddd, J=11.4, 5.7, 5.7 Hz, 1H); 1.94-1.86 (m, 3H); 1.66 (d, J=6.7 Hz, 3H).

### Example 48

### 3-(S)-Cyclobutylaminomethyl-2-(R)-(4-fluoro-2-methyl-phenyl)-pyrrolidine-1-carboxylic acid [1-(R)-(3,5-bis-trifluoromethyl-phenyl)-ethyl]-methyl-amide

LRMS m/z (APCI⁺) 560 [M+H]; 500 MHz ¹HNMR (CD₃OD) δ 7.87 (s, 1H); 7.70 (s, 2H); 7.41 (dd, J=7.3, 7.3 Hz, 1H); 6.93-6.90 (m, 2H); 5.29 (q, J=6.5 Hz, 1H); 4.93 (d, 1H); 3.99 (ddd, J=8.8, 8.8, 0.0 Hz, 1H); 3.76-3.68 (m, 2H); 3.09 (ddd, J=12.4, 12.4, 0.0 Hz, 1H); 2.83 (dd, J=12.5, 0.0 Hz, 1H); 2.52 (s, 3H); 2.47 (s, 3H); 2.52-2.47 (m, 1H), 2.38 (ddd, J=11.4, 5.7, 5.7 Hz, 1H); 2.30-2.24 (m, 2H); 2.13 (dddd, J=18.7, 9.3, 9.3, 9.3 Hz, 2H); 1.94-1.84 (m, 3H); 1.66 (d, J=6.7 Hz, 3H).

### Example 49

### 2-(R)-(4-Fluoro-2-methyl-phenyl)-3-(S)-{[3-(2-oxo-pyrrolidin-1-yl)-propylamino]-methyl}-pyrrolidine-1-carboxylic acid [1-(R)-(3,5-bis-trifluoromethyl-phenyl)-ethyl]-methyl-amide

LRMS m/z (APCI⁺) 631 [M+H]; 500 MHz ¹HNMR (CD₃OD) δ 7.88 (s, 1H); 7.70 (s, 2H); 7.42 (dd, J=7.8, 7.8 Hz, 1H); 6.93-6.90 (m, 2H); 5.30 (q, J=6.9 Hz, 1H); 4.94 (s, 1H); 4.00 (ddd, J=10.4, 10.4 6.2 Hz, 1H); 3.71 (ddd, J=9.3, 9.3, 0.0 Hz, 1H); 3.50-3.35 (m, 4H); 3.18 (dddd, J=11.9, 11.9, 0.0, 0.0 Hz, 1H); 3.01-2.97 (m, 3H); 2.53 (s, 3H); 2.53-2.48 (m, 1H); 2.48 (s, 3H); 2.46-2.40 (m, 3H); 2.08 (dddd, J=7.8, 7.8, 7.8, 7.8 Hz, 2H); 1.93-1.87 (m, 3H); 1.66 (d, J=7.3 Hz, 3H).

### Example 50

### 3-(S)-(3-Ethoxy-propylamino)-methyl]-2-(R)-(4-fluoro-2-methyl-phenyl)-pyrrolidine-1-carboxylic acid [1-(R)-(3,5-bis-trifluoromethyl-phenyl)-ethyl]-methyl-amide

LRMS m/z (APCI⁺) 592 [M+H]; 500 MHz ¹HNMR (CD₃OD) δ 7.88 (s, 1H); 7.70 (s, 2H); 7.41 (dd, J=7.8, 7.8 Hz, 1H); 6.93-6.90 (m, 2H); 5.30 (q, J=6.9 Hz, 1H); 4.93 (d, J=9.9 Hz, 1H); 4.00 (ddd, J=10.4, 10.4, 6.3 Hz, 1H); 3.70 (ddd, J=8.8, 8.8, 0.0 Hz, 1H); 3.53 (t, J=5.5 Hz, 2H); 3.49 (q, J=7.1 Hz, 2H); 3.21 (dddd, J=11.9, 11.9, 0.0, 0.0 Hz, 1H); 3.12 (t, J=7.3 Hz, 2H); 3.02 (ddd, J=11.9, 2.6, 0.0 Hz, 1H); 2.52 (s, 3H); 2.48 (s, 3H); 2.55-2.48 (m, 1H); 2.37 (ddd, J=11.9, 6.2, 6.2 Hz, 1H); 1.95-1.84 (m, 3H); 1.66 (d, J=6.7 Hz, 3H); 1.16 (dddd, J=7.3, 7.3, 0.0 Hz, 3H).

### Example 51

### 2-(R)-(4-Fluoro-2-methyl-phenyl)-3-(S)-[(2-hydroxy-1-methyl-ethylamino)-methyl]-pyrrolidine-1-carboxylic acid [1-(R)-(3,5-bis-trifluoromethyl-phenyl)-ethyl]-methyl-amide

LRMS m/z (APCI⁺) 564 [M+H]; 500 MHz ¹HNMR (CD₃OD) δ 7.88 (s, 1H); 7.70 (s, 2H); 7.42 (dd, J=7.3, 7.3 Hz, 1H); 6.93-6.90 (m, 2H); 5.30 (q, J=6.6 Hz, 1H); 4.94 (d, J=9.9 Hz, 1H); 3.99 (ddd, J=16.6, 16.6, 10.4 Hz, 1H); 3.76 (ddd, J=12.4, 3.6, 0.0 Hz, 1H); 3.70 (ddd, J=8.8, 8.8, 0.0 Hz, 1H); 3.55 (ddd, J=12.4, 2.1, 2.1 Hz, 1H); 3.36-3.32 (m, 2H); 3.26 (dddd, J=12.4, 12.4, 0.0 Hz, 1H); 3.03 (dd, J=11.9, 0.0 Hz, 1H); 2.53 (s, 3H); 2.49 (s, 3H); 2.53-2.49 (m, 1H); 2.45-2.42 (m, 1H); 1.89 (dddd, J=10.9, 10.9, 10.9, 10.9 Hz, 1H); 1.66 (d, J=6.7 Hz, 3H); 1.28 (d, J=6.7 Hz, 3H).

### Example 52

### 2-(R)-(4-Fluoro-2-methyl-phenyl)-3-(S)-[(1-hydroxymethyl-propylamino)-methyl]-pyrrolidine-1-carboxylic acid [1-(R)-(3,5-bis-trifluoromethyl-phenyl)-ethyl]-methyl-amide

LRMS m/z (APCI⁺) 578 [M+H]; 500 MHz ¹HNMR (CD₃OD) δ 7.88 (s, 1H); 7.70 (s, 2H); 7.42 (dd, J=7.3, 7.3 Hz, 1H); 6.93-6.90 (m, 2H); 5.30 (q, J=6.9 Hz, 1H); 4.94 (d, J=9.9 Hz, 1H); 3.99 (ddd, J=10.4, 10.4, 5.7 Hz, 1H); 3.78 (ddd, J=12.4, 3.1, 0.0 Hz, 1H); 3.70 (ddd, J=8.8, 8.8, 0.0 Hz, 1H); 3.64 (ddd, J=12.4, 5.7, 0.0 Hz, 1H); 3.24 (ddd, J=12.4, 12.4, 0.0 Hz, 1H); 3.12-3.06 (m, 2H); 2.57-2.49 (m, 1H); 2.53 (s, 3H); 2.49 (s, 3H); 2.44 (ddd, J=11.9, 6.2, 0.0 Hz, 1H); 1.89 (dddd, J=11.4, 11.4, 11.4, 11.4 Hz, 1H); 1.69-1.63 (m, 5H); 0.97 (t, J=7.6 Hz, 3H).

### Example 53

### 3-(S)-[(Cyclopropylmethyl-amino)-methyl]-2-(R)-(4-fluoro-2-methyl-phenyl)-pyrrolidine-1-carboxylic acid [1-(R)-(3,5-bis-trifluoromethyl-phenyl)-ethyl]-methyl-amide

LRMS m/z (APCI⁺) 560 [M+H]; 500 MHz ¹HNMR (CD₃OD) δ 7.88 (s, 1H); 7.70 (s, 2H); 7.42 (dd, J=7.3, 7.3 Hz, 1H); 6.93-6.90 (m, 2H); 5.30 (q, J=6.7 Hz, 1H); 4.92 (s, 1H); 4.00 (ddd, J=10.4, 10.4, 5.7 Hz, 1H); 3.70 (ddd, J=8.3, 8.3, 0.0 Hz, 1H); 3.20 (ddd, J=11.9, 11.9, 0.0 Hz, 1H); 3.03-2.97 (m, 2H); 2.82 (ddd, J=12.4, 7.8, 0.0 Hz, 1H); 2.52-2.48 (m, 1H); 2.52 (s, 3H); 2.48 (s, 3H); 2.46-2.39 (m, 1H); 1.89 (dddd, J=11.4, 11.4, 11.4, 11.4 Hz, 1H); 1.66 (d, J=7.3 Hz, 3H); 1.04 (dddd, J=7.8, 7.8, 5.2, 5.2 Hz, 1H); 0.68-0.66 (m, 2H); 0.36-0.35 (m, 2H).

### Experimental Procedure for Examples 54-123 (Table 2)

The compounds listed in Table 2 were prepared from Intermediate 24. The amines for sidechain “Z” (0.2 mmol, 4.0 eq) were pre-weighed in 1 dram, septa-capped vials. The aldehyde (0.05 mmol, 1.0 eq) was dissolved in 1 mL of anhydrous THF and added to the reaction vials and the resulting solutions shaken at rt for 16 h. To each reaction vial was then added Na(OAc)₃BH (30.00 mg, 2.5 eq) and the vials were shaken and addtitional 4.5 h. The reactions were quenched by adding 1N NaOH and 2.25 mL EtOAc. The organics were separated and loaded onto an equilibrated SCX SPE (conditioned with 5 mL MeOH, 2×5 mL EtOAc) columns. The desired products were then eluted off using 1N TEA in MeOH (5 mL). These solutions were collected in tared vials and dried under a N₂ stream. Purifications were accomplished by HPLC separation on a Waters Symmetry C¹⁸ column (5 mm, 3.9×150 mm) with a 1.0 mL/min flow rate eluting with a gradient system of 100%, 80%, 0% (0.1% TFA in *(H₂O/CH₃CN) injecting each sample in 2 mL of solvent. Isolated and tested as the TFA salts.

TABLE 2

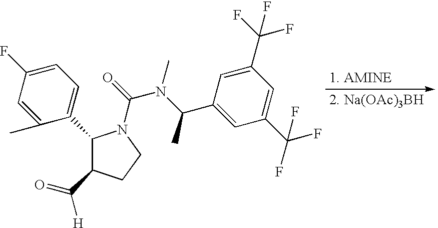

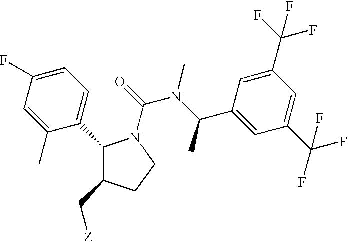

| Example | Z | m/z [M + H] |
|---|---|---|
| 54 | 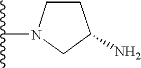 | 575 |
| 55 | 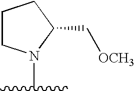 | 604 |
| 56 | 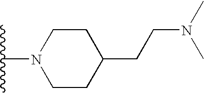 | 645 |
| 57 | 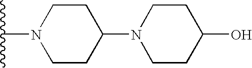 | 673 |
| 58 | 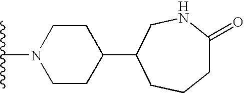 | 686 |
| 59 | 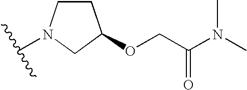 | 661 |
| 60 | 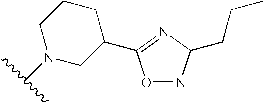 | 684 |
| 61 | 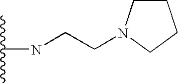 | 603 |
| 62 | 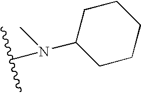 | 602 |
| 63 | 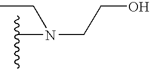 | 578 |
| 64 | 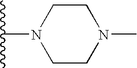 | 589 |
| 65 | 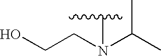 | 592 |
| 66 | 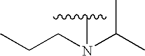 | 576 |
| 67 | 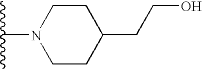 | 618 |
| 68 | 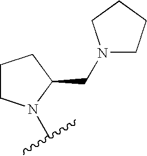 | 643 |
| 69 | 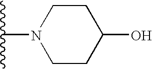 | 590 |
| 70 | 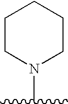 | 574 |
| 71 | 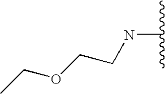 | 578 |
| 72 | 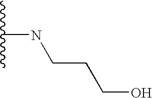 | 564 |
| 73 | 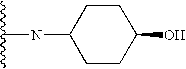 | 604 |
| 74 | 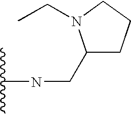 | 617 |
| 75 | 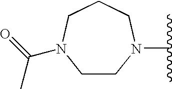 | 631 |
| 76 | 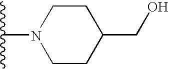 | 604 |
| 77 | 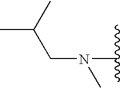 | 576 |
| 78 | 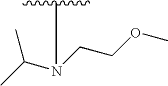 | 606 |
| 79 | 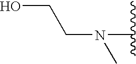 | 564 |
| 80 | 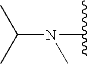 | 562 |
| 81 | 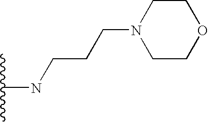 | 633 |
| 82 | 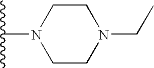 | 603 |
| 83 | 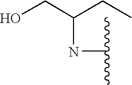 | 576 |
| 84 | 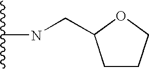 | 590 |
| 85 | 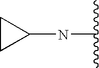 | 546 |
| 86 | 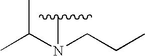 | 592 |
| 87 | 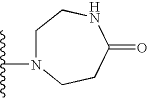 | 603 |
| 88 | 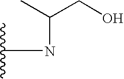 | 564 |
| 89 | 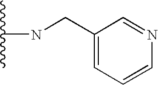 | 597 |
| 90 | 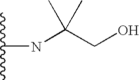 | 578 |
| 91 | 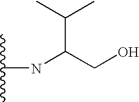 | 592 |
| 92 | 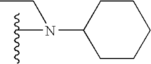 | 616 |
| 93 | 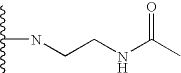 | 591 |
| 94 | 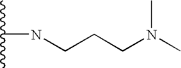 | 591 |
| 95 | 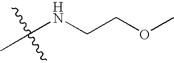 | 564 |
| 96 | 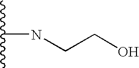 | 550 |
| 97 | 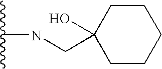 | 618 |
| 98 | 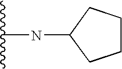 | 574 |
| 99 | 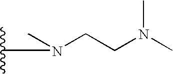 | 591 |
| 100 | 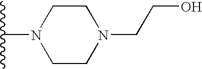 | 619 |
| 101 | 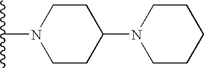 | 657 |
| 102 | 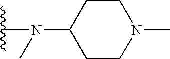 | 617 |
| 103 | 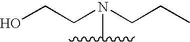 | 592 |
| 104 | 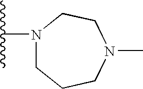 | 603 |
| 105 | 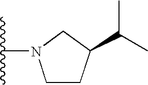 | 603 |
| 106 | 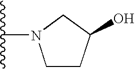 | 576 |
| 107 | 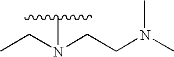 | 605 |
| 108 | 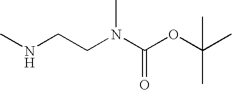 | 677 |
| 109 | 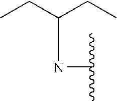 | 576 |
| 110 | 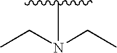 | 562 |
| 111 | 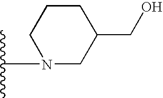 | 604 |
| 112 | 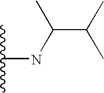 | 576 |
| 113 | 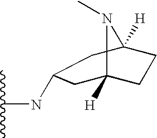 | 629 |
| 114 | 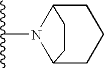 | 600 |
| 115 | 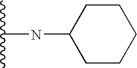 | 588 |
| 116 | 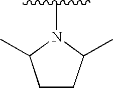 | 588 |
| 117 | 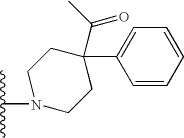 | 692 |
| 118 | 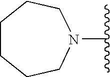 | 588 |
| 119 | 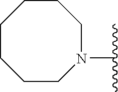 | 602 |
| 120 | 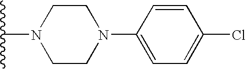 | 686 |
| 121 | 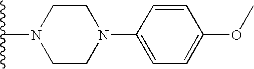 | 681 |
| 122 | 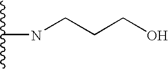 | 564 |
| 123 | 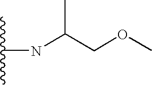 | 578 |

### Experimental Procedure for Examples 124-173 (Table 3)

The compounds listed in Table 3 were prepared from Intermediate 25. The amines (for sidechain Z) (0.2 mmol, 4.0 eq) were pre-weighed in 1 dram, septa-capped vials. The aldehyde (0.1 mmol, 1.0 eq, 50.4 mg) was dissolved in 1 mL of anhydrous THF and added to the reaction vials and the resulting solutions shaken at rt for 16 h. To each reaction vial was added Na(OAc)₃BH (2.0 eq, 0.2 mmol, 50.0 mg) and the vials were shaken and addtitional 6 h. The reactions were quenched by adding 2.4 mL 1N NaOH and 2.4 mL EtOAc. The organics were separated and loaded onto an equilibrated SCX SPE (conditioned with 5 mL MeOH, 2×5 mL EtOAc) columns. The desired products were then eluted off using 1N TEA in MeOH (5 mL). These solutions were collected in tared vials and dried under a N₂ stream. Purifications were accomplished by HPLC separation on a Waters Symmetry C¹⁸ column (5 mm, 3.9×150 mm) with a 1.0 mL/min flow rate eluting with a gradient system of 100%, 80%, 0% (0.1% TFA in H₂O/CH₃CN) injecting each sample in 2 mL of solvent. Isolated and tested as the TFA salts.

TABLE 3

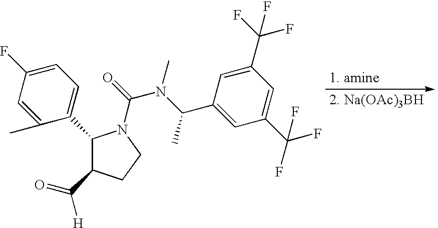

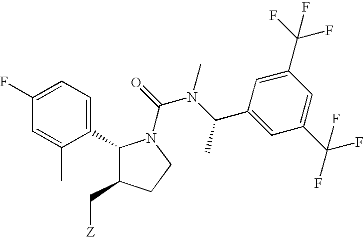

| Example | Z | m/z [M + H] |
|---|---|---|
| 124 | 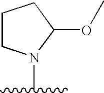 | 604 |
| 125 | 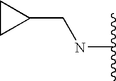 | 546 |
| 126 | 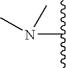 | 534 |
| 127 | 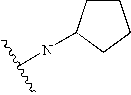 | 574 |
| 128 | 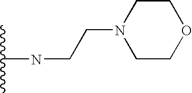 | 619 |
| 129 | 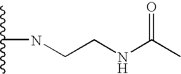 | 591 |
| 130 | 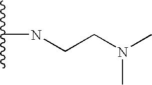 | 577 |
| 131 | 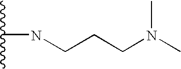 | 591 |
| 132 | 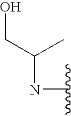 | 564 |
| 133 | 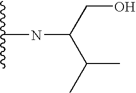 | 592 |
| 134 | 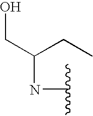 | 578 |
| 135 | 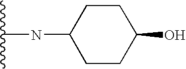 | 604 |
| 136 | 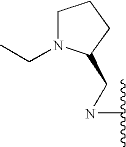 | 617 |
| 137 | 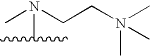 | 591 |
| 138 | 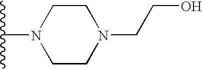 | 619 |
| 139 | 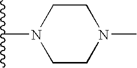 | 589 |
| 140 | 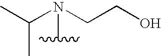 | 592 |
| 141 | 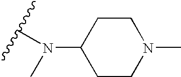 | 617 |
| 142 | 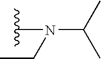 | 576 |
| 143 | 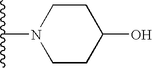 | 590 |
| 144 | 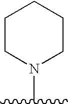 | 574 |
| 145 | 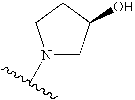 | 576 |
| 146 | 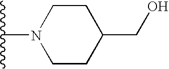 | 604 |
| 147 | 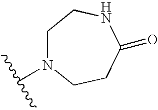 | 603 |
| 148 | 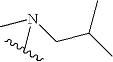 | 576 |
| 149 | 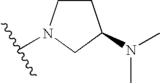 | 603 |
| 150 | 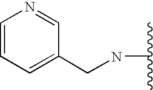 | 597 |
| 151 | 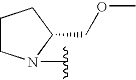 | 604 |
| 152 | 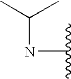 | 548 |
| 153 | 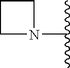 | 546 |
| 154 | 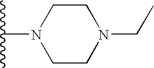 | 603 |
| 155 | 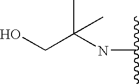 | 578 |
| 156 | 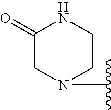 | 589 |
| 157 | 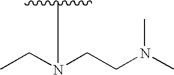 | 605 |
| 158 | 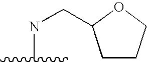 | 590 |
| 159 | 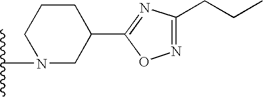 | 684 |
| 160 | 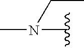 | 548 |
| 161 | 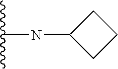 | 590 |
| 162 | 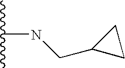 | 560 |
| 163 | 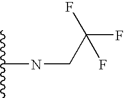 | 588 |
| 164 | 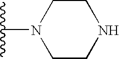 | 575 |
| 165 | 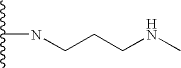 | 577 |
| 166 | 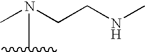 | 577 |
| 167 | 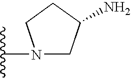 | 575 |
| 168 | 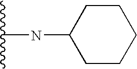 | 588 |
| 169 | 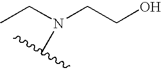 | 578 |
| 170 | 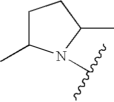 | 588 |
| 171 | 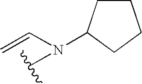 | 614 |
| 172 | 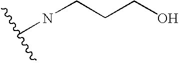 | 564 |
| 173 | 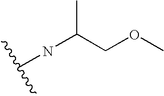 | 578 |

### Experimental Procedure for Examples 174-211 (Table 4)

The compounds listed in Table 4 were prepared from the known compound 2-o-Tolyl-pyrrolidine (J. Med. Chem.; EN; 33; 10; 1990; 2793-2797). The pyrrolidine (0.66 g, 4.10 mmol) was dissolved in 20.0 mL of anhydrous CH₂Cl₂ and Et₃N (2.23 mL, 16.0 mmol) was added. Triphosgene (0.42 g, 1.39 mmol) was separately dissolved in an additional 20.0 mL of anhydrous CH₂Cl₂ and added dropwise to the reaction solution under N₂. The reaction was stirred at rt for 1½ h.

To the varying preweighed amines (for sidechain Z) was added N,N,N-diisopropylethylamine (11.7 uL, 0.07 mmol), followed by 520 uL of the carbamoyl chloride solution formed above. The sealed vials were then placed on a heater/shaker plate at 42° C. for 16 h. The reactions were quenched with PS-isocyanate (0.10 g, 1.47 mmol). The vials were then shaken for an additional hour at rt. Each vial was then transferred onto a 6 mL fritted barrel to filter off resin. The reaction solutions were collected into separate vials. These suspensions were further separated by additing 0.5 mL of 3 N NaOH aqueous solution to each vial and separating the organic layers to be evaporated in the Savant. Purifications were accomplished by HPLC separation on a Waters Symmetry C¹⁸ column (5 mm, 3.9×150 mm) with a 1.0 mL/min flow rate eluting with a gradient system of 100%, 80%, 0% (0.1% TFA in H₂O/CH₃CN) injecting each sample in 2 mL of solvent. Isolated and tested as the TFA salts.

TABLE 4

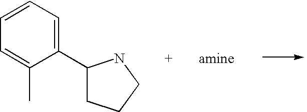

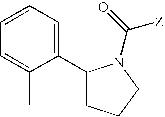

| Example | Z | m/z [M + H] |
|---|---|---|
| 174 | 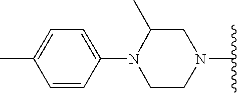 | 377 |
| 175 | 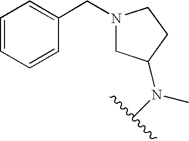 | 377 |
| 176 | 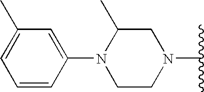 | 377 |
| 177 | 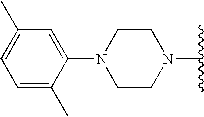 | 377 |
| 178 | 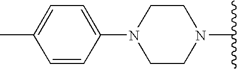 | 363 |
| 179 | 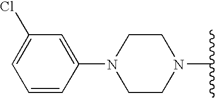 | 383 |
| 180 | 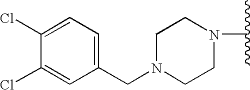 | 431 |
| 181 | 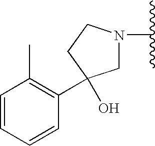 | 364 |
| 182 | 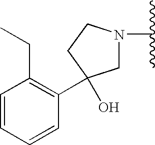 | 378 |
| 183 | 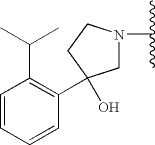 | 392 |
| 184 | 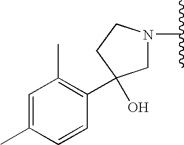 | 378 |
| 185 | 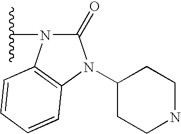 | 404 |
| 186 | 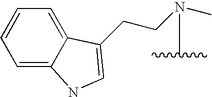 | 361 |
| 187 | 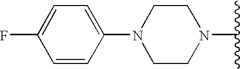 | 367 |
| 188 | 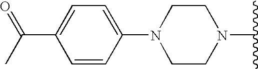 | 391 |
| 189 | 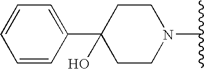 | 364 |
| 190 | 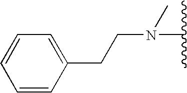 | 322 |
| 191 | 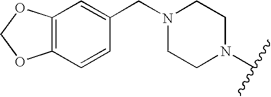 | 407 |
| 192 | 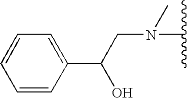 | 338 |
| 193 | 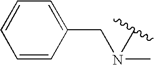 | 365 |
| 194 | 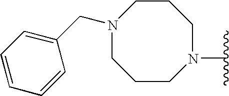 | 377 |
| 195 | 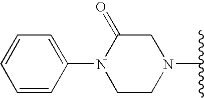 | 363 |
| 196 | 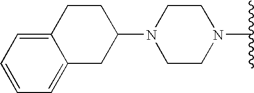 | 404 |
| 197 | 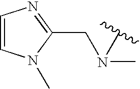 | 312 |
| 198 | 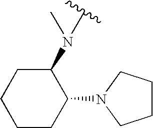 | 369 |
| 199 | 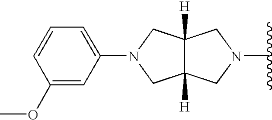 | 405 |
| 200 | 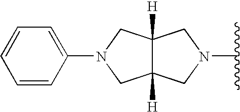 | 375 |
| 201 | 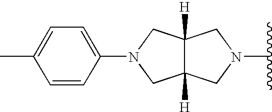 | 389 |
| 202 | 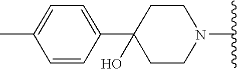 | 378 |
| 203 | 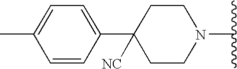 | 373 |
| 204 | 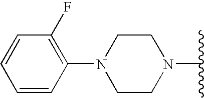 | 367 |
| 205 | 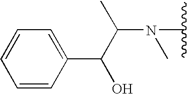 | 352 |
| 206 | 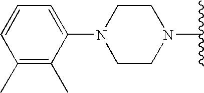 | 377 |
| 207 | 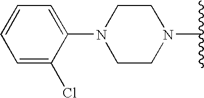 | 383 |
| 208 | 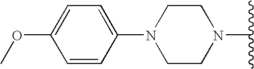 | 379 |
| 209 | 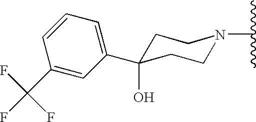 | 432 |
| 210 | 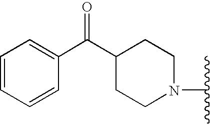 | 476 |
| 211 | 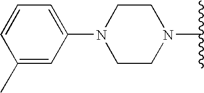 | 363 |

### Example 212

### 2-o-Tolyl-pyrrolidine-1-carboxylic acid [1-(3,5-bis-trifluoromethyl-phenyl)-ethyl]-methyl-amide

To the known compound 2-o-tolyl-pyrrolidine (J. Med. Chem.; EN; 33; 10; 1990; 2793-2797) (0.20 g, 1.24 mmol) in 10 mL of anhydrous CH₂Cl₂, was added Et₃N (0.67 mL, 4.84 mmol). Triphosgene (0.12 g, 0.41 mmol) was separately dissolved in CH₂Cl₂ and added dropwise to the reaction. The resulting suspension was stirred at rt for 1½ h. The racemic form of intermediate 20 (0.38 g, 1.24 mmol) was added to the reaction, followed by diisopropylethylamine (0.29 mL, 1.66 mmol) and the reaction was heated to reflux in an oil bath at 45° C. for 21 h. The reaction was cooled to rt and diluted with CH₂Cl₂, washed with 1M HCl (20 mL) and extracted. Combined organics were washed with brine, dried over Na₂SO₄, filtered and concentrated under reduced pressure to give a brown oil. Purification was accomplished by flash chromatography on a 35 g Isco silica gel column eluting with a gradient system of 2%, 5%, 8%, 10% MeOH/CH₂Cl₂ and collecting 13 mm fractions. Product containing fractions (11-32) were combined and concentrated under reduced pressure to give a colorless foam (0.28 g, 0.60 mmol, 48% yield); Rf 0.80 (10% MeOH/CH₂Cl₂); LRMS m/z (APCI⁺) 459 [M+H]; 500 MHz ¹HNMR (CDCl₃) δ diagnostic peak of benzylic hydrogen at 4.93 (d, J=7.1 Hz, 1H) of trans isomer in a 2:1 ratio with the cis isomer; 4.89 (d, J=7.9 Hz, 1H).

### Example 213

### 3-Amino-2-(4-fluoro-2-methyl-phenyl)-piperidine-1-carboxylic acid [1-(3,5-bis-trifluoromethyl-phenyl)-ethyl]-methyl-amide (more polar isomer)

Intermediate 33 (0.042 g, 0.069 mmol) was dissolved in 5 mL of anhydrous CH₂Cl₂ under N₂. Trifluoroacetic acid (TFA, 0.053 mL, 0.690 mmol) was added dropwise and the reaction stirred at rt for 15 h. The reaction was diluted in EtOAc (20 mL) and extracted with aqueous saturated NaHCO₃ (3×20 mL). Combined organics were then dried over Na₂SO₄, filtered and concentrated to give a colorless oil. This material was used directly to make the HCl salt to give a colorless crystalline solid (0.025 g, 0.050 mmol, 71% yield); Rf (0.45 10% MeOH/CH₂Cl₂); LRMS m/z (APCI⁺) 505 [M+H].

### Example 214

### 3-Amino-2-(4-fluoro-2-methyl-phenyl)-piperidine-1-carboxylic acid [1-(3,5-bis-trifluoromethyl-phenyl)-ethyl]-methyl-amide (less polar isomer)

Using intermediate 32, the same procedure was followed as for example 213. The product was obtained as a colorless crystalline HCl salt (15.5 mg, 0.03 mmol, 63% yield); Rf 0.50 (10% MeOH/CH₂Cl₂); LRMS m/z (APCI⁺) 505 [M+H].

### Example 215

### 2-(4-Fluoro-2-methyl-phenyl)-3-methylamino-piperidine-1-carboxylic acid [1-(3,5-bis-trifluoromethyl-phenyl)-ethyl]-methyl-amide

Using intermediate 32, the same procedure was followed as for example 213. The product was obtained as a colorless oil (68.0 mg, 0.13 mmol, 92% yield); Rf 0.70 (10% MeOH/CH₂Cl₂); LRMS m/z (APCI⁺) 520 [M+H].

### Example 216

### 3-Dimethylamino-2-(4-fluoro-2-methyl-phenyl)-piperidine-1-carboxylic acid [1-(3,5-bis-trifluoromethyl-phenyl)-ethyl]-methyl-amide

Example 215 (0.03 g, 0.06 mmol) was dissolved in 2 mL of anhydrous THF under N₂. Formaldehyde (37% in H₂O, 2.00 uL, 0.29 mmol) was added, followed by addition of Na(OAc)₃BH (0.06 g, 0.29 mmol) and the reaction was stirred at rt for 1 hr. The reaction was then quenched with saturated aqueous NaHCO₃ and extracted with EtOAc (3×15 mL). The combined organics were dried over Na₂SO₄, filtered and concentrated under reduced pressure to give a colorless oil (0.03 g, 0.60 mmol). Purification was accomplished by flash chromatography on a 10 g Isco silica gel column eluting with a gradient system of 2%, 4%, 6% MeOH/CH₂Cl₂ and collecting 8 mL fractions. Product containing fractions (28-48) were combined and concentrated under reduced pressure to give a colorless oil (9.00 mg, 0.02 mmol, 30% yield); Rf 0.65 (10% MeOH/CH₂Cl₂); LRMS m/z (APCI⁺) 534 [M+H].

### Example 217

### 2-(4-Fluoro-2-methyl-phenyl)-3-methylamino-piperidine-1-carboxylic acid [1-(3,5-bis-trifluoromethyl-phenyl)-ethyl]-methyl-amide

Using intermediate 33, the same procedure was followed as for example 213. The desired product obtained as a colorless oil (76.0 mg, 0.146 mmol, quantitative yield); Rf 0.25 (10% MeOH/CH₂Cl₂); LRMS m/z (APCI⁺) 520 [M+H].

### Example 218

### 2-(4-Fluoro-2-methyl-phenyl)-3-methylamino-piperidine-1-carboxylic acid [1-(3,5-bis-trifluoromethyl-phenyl)-ethyl]-methyl-amide

Same as example 217, but free-base form.

### Example 219

### 3-Dimethylamino-2-(4-fluoro-2-methyl-phenyl)-piperidine-1-carboxylic acid [1-(3,5-bis-trifluoromethyl-phenyl)-ethyl]-methyl-amide

Using example 217, the same procedure was followed as for example 216. The product was obtained as a colorless oil (60.0 mg, 0.11 mmol, 92% yield); Rf 0.55 (10% MeOH/CH₂Cl₂); LRMS m/z (APCI⁺) 534 [M+H].

### Example 220

### 2-(R)-(4-Fluoro-2-methyl-phenyl)-3-(S)-methylaminomethyl-piperidine-1-(R)-carboxylic acid [1-(3,5-bis-trifluoromethyl-phenyl)-ethyl]-methyl-amide

Starting aldehyde, intermediate 39 (0.15 g, 0.29 mmol) was dissolved in 7.24 mL (14.48 mmol) of 2M methylamine in MeOH and stirred at rt for 1 h under N₂. NaBH₄ (10.90 mg, 0.29 mmol) was then added and the reaction stirred for an additional 30 minutes. The crude material was concentrated under reduced pressure to give a brown oil: Purification was accomplished through flash chromatography on a 10 g Isco silica gel column and eluting with 5% MeOH/CH₂Cl₂ with 0.2% NH₄OH and collected 8 mL fractions. Product containing fractions were combined to give a viscous oil (0.105 g, 0.197 mmol, 68% yield); Rf 0.25 (10% MeOH/CH₂Cl₂+0.2% NH₄OH); LRMS m/z (APCI⁺) 534 [M+H].

### Example 221

### 3-(S)-Dimethylaminomethyl-2-(R)-(4-fluoro-2-methyl-phenyl)-piperidine-1-(R)-carboxylic acid [1-(3,5-bis-trifluoromethyl-phenyl)-ethyl]-methyl-amide

Example 220 (42.0 mg, 0.1 mmol) was dissolved in 1 mL of anhydrous THF. A 37% aqueous solution of formaldehyde (38.0 uL, 14.1 mg, 0.6 mmol) was added, followed by Na(OAc)₃BH (0.1 g, 0.5 mmol) and the reaction was stirred at rt for 16 h. The crude material was concentrated under reduced pressure to give a viscous oil. Purification was accomplished via flash chromatography on a 4 g Isco silica gel column using 5% MeOH/CH₂Cl₂ with 0.2% NH₄OH as eluent and collected 8 mL fractions. Product containing fractions were combined to give a viscous gum (25.0 mg, 0.1 mmol, 58% yield); Rf 0.5 (10% MeOH/CH₂Cl₂ with 0.2% NH₄OH); LRMS m/z (APCI⁺) 548 [M+H].

### Example 222

### Cis-3-Amino-2-phenyl-piperidine-1-carboxylic acid [1-(3,5-bis-trifluoromethyl-phenyl)-ethyl]-methyl-amide (less polar isomer)

Using example 41 (67.00 mg, 0.12 mmol) was dissolved in 2 mL of anhydrous DCE. Et₃SiH (186 uL, 1.17 mmol) was added to the solution, followed by TFA (180 uL, 2.34 mmol) and the reaction was then heated to 75° C. for 1 h. The solution was then cooled and concentrated under reduced pressure. The residual oil was then partitioned between CH₂Cl₂ (10 mL) and saturated NaHCO₃ aqueous solution (10 mL). The organics were extracted and dried over Na₂SO₄, filtered and concentrated under reduced pressure to give a crude oil. Purification was accomplished through flash chromatography on a 10 g Isco silica gel column using 5% MeOH/CH₂Cl₂ and collected 8 mL fractions. Product containing fractions were combined and concentrated under reduced pressure to give the desired material (27.0 mg, 0.057 mmoles, 49% yield); Rf 0.4 (10% MeOH/CH₂Cl₂); LRMS m/z (APCI⁺) 474 [M+H]; 500 MHz ¹HNMR (CDCl₃) δ 7.76 (s, 1H); 7.63 (s, 2H); 7.23-7.19 (m, 5H); 5.56 (q, J=7.1 Hz, 1H); 4.42 (d, J=3.3 Hz, 1H); 3.32 (ddd, J=8.6, 4.2, 4.2 Hz, 1H); 3.09 (ddd, J=12.9, 9.5, 2.9 Hz, 1H); 2.95 (ddd, J=12.9, 9.5, 2.9 Hz, 1H); 2.83 (s, 3H); 1.95-1.66 (m, 6H); 1.52 (d, J=7.1 Hz, 3H).

### Example 223

### Cis-3-Amino-2-phenyl-piperidine-1-carboxylic acid [1-(3,5-bis-trifluoromethyl-phenyl)-ethyl]-methyl-amide (more polar)

Using example 42, the same procedure was followed as for example 222. The product was obtained as a colorless oil (26.0 mg, 0.06 mmol, 41% yield); Rf 0.35 (10% MeOH/CH₂Cl₂); LRMS m/z (APCI⁺) 474 [M+H]; 500 MHz ¹HNMR (CDCl₃) δ 7.77 (s, 1H); 7.65 (s, 2H); 7.34-7.19 (m, 5H); 5.43 (q, J=6.9 Hz, 1H); 4.56 (d, J=3.7 Hz, 1H); 3.32-3.26 (m, 1H); 3.14-3.08 (m, 2H); 2.74 (s, 3H); 1.97-1.89 (m, 2H); 1.86-1.65 (m, 4H); 1.52 (d, J=7.1 Hz, 3H).

### Example 224

### {1-[(3,5-Bis-trisfluoromethyl-benzyl)-methyl-carbamoyl]-2-(R)-phenyl-piperidin-3-(R)-yl}-methyl-carbamic acid tert-butyl ester

Intermediate 43 (0.30 g, 0.55 mmol) was dissolved in 5.5 mL of anhydrous THF and 171 uL of MeI (0.39 g, 2.75 mmol). Solid NaH (66.0 mg, 2.75 mmol) was added and the reaction was stirred under N₂ at rt for 1 h. The reaction was then quenched with 5 mL of saturated aqueous NaHCO₃ solution and extracted with CH₂Cl₂ (3×10 mL). Combined organics were then dried over MgSO₄, filtered and concentrated under reduced pressure. Purification was accomplished through flash chromatography on a 10 g isco silica gel column using 20% EtOAc/Hexanes and collected 8 mL fractions. Product containing fractions were combined to obtain the desired material as colorless solid (0.32 g, 0.55 mmol, 100% yield); Rf 0.5 (50% EtOAc/Hexanes); LRMS m/z (APCI⁺) 574/474 [M+H].

### Example 225

### 3-(R)-Methylamino-2-(R)-phenyl-piperidine-1-carboxylic acid (3,5-bis-trifluoromethyl-benzyl)-methyl-amide

Using example 224, the same procedure was followed as for example 222. The desired product was then redissolved in THF and made into the mono-HCl salt using 1 mL of 2N HCl/Et₂O and isolated as a colorless solid (0.23 g, 0.45 mmol, 89% yield); LRMS m/z (APCI⁺) 474 [M+H]; 500 MHz ¹HNMR (CD₃OD) δ 7.88 (s, 1H); 7.78 (s, 2H); 7.39-7.36 (m, 5H); 4.84 (d, J=4.2 Hz, 1H); 4.50 (dddd, J=15.3, 15.3, 15.3, 0.0 Hz, 2H); 3.63 (ddd, J=7.1, 3.7, 3.7 Hz, 1H); 3.56-3.51 (m, 1H); 3.12 (ddd, J=12.0, 8.7, 3.7 Hz, 1H); 3.06 (s, 3H); 2.48 (s, 3H); 2.30-2.21 (m, 1H); 2.14-2.04 (m, 1H); 1.92-1.70 (m, 2H).

### Example 226

### 3-(R)-Dimethylamino-2-(R)-phenyl-piperidine-1-carboxylic acid (3,5-bis-trifluoromethyl-benzyl)-methyl-amide

Using the free base of example 225, the same procedure was followed as for example 221. The desired product was obtained as a colorless solid (47.0 mg, 0.10 mmol, 91% yield); Rf 0.7 (10% MeOH/CH₂Cl₂); LRMS m/z (APCI⁺) 488 [M+H].

### Example 227

### (1-{[1-(3,5-Bis-trifluoromethyl-phenyl)-ethyl]-methyl-carbamoyl}-2-(R)-phenyl-piperidin-3-(R)-yl)-methyl-carbamic acid tert-butyl ester (less polar)

Using intermediate 41, the same procedure was followed as for example 224. Desired product was obtained as a colorless solid (0.532 g, 0.905 mmol, 86% yield); Rf, 0.7 (40% EtOAc/Hexanes); LRMS m/z (APCI⁺) 588/488 [M+H]; 500 MHz ¹HNMR (CDCl₃) δ 7.68 (s, 1H); 7.55 (s, 2H); 7.27-7.20 (m, 5H); 5.15 (d, J=6.2 Hz, 1H); 5.06 (q, J=6.8 Hz, 1H); 4.30 (bs, 1H); 3.70-3.61 (m, 2H); 2.56 (s, 3H); 2.00 (bs, 4H); 1.84-1.68 (m, 3H); 1.53 (d, J=6.6 Hz, 3H); 1.44 (s, 9H).

### Example 228

### (1-{[1-(3,5-Bis-trifluoromethyl-phenyl)-ethyl]-methyl-carbamoyl}-2-(R)-phenyl-piperidin-3-(R)-yl)-methyl-carbamic acid tert-butyl ester (more polar)

Using intermediate 42, the same procedure was followed as for example 224. The desired material was obtained as a pale yellow solid (0.23 g, 0.39 mmol, 36% yield); Rf 0.7 (40% EtOAc/Hexanes); LRMS m/z (APCI⁺) 588/488 [M+H]; 500 MHz ¹HNMR (CDCl₃) δ 7.74 (s, 1H); 7.72 (s, 2H); 7.27-7.22 (m, 5H); 5.09 (bs, 2H); 4.30 (bs, 1H); 3.71-3.57 (m, 2H); 2.56 (s, 3H); 2.03-2.00 (m, 4H); 1.80-1.68 (m, 3H); 1.45-1.38 (m, 12H).

### Example 229

### 3-(S)-Amino-2-(S)-phenyl-piperidine-1-carboxylic acid [1-(3,5-bis-trifluoromethyl-phenyl)-ethyl]-methyl-amide (less polar)

Using intermediate 45, the same procedure was followed as for example 222. The desired material was obtained as viscous oil and converted to the mono HCl salt in EtOAc, using 65 uL 2N HCl/Et₂O and recrystallized with isopropyl ether to give a colorless solid (42.0 mg, 0.08 mmol, 63% yield); LRMS m/z (APCI⁺) 474 [M+H]; 500 MHz ¹HNMR (CD₃OD) δ 7.90 (s, 1H); 7.79 (s, 2H); 7.40-7.30 (m, 5H); 5.40 (q, J=6.9 Hz, 1H); 4.66 (d, J=3.3 Hz, 1H); 3.74 (bs, 1H); 3.51-3.48 (m, 1H); 3.12-3.07 (m, 1H); 2.91 (s, 3H); 2.19-2.14 (m, 1H); 2.10-2.04 (m, 1H); 1.89-1.79 (m, 2H); 1.58 (d, J=7.1 Hz, 3H).

### Example 230

### 3-(S)-Amino-2-(S)-phenyl-piperidine-1-carboxylic acid [1-(3,5-bis-trifluoromethyl-phenyl)-ethyl]-methyl-amide (more polar)

Using intermediate 46, the same procedure was followed as for example 222. The product was converted to the mono-HCl salt in 2 mL of EtOAc, using 70 uL of 2 N HCl/Et₂O and recrystallized with isopropyl ether to give the desired material as a colorless solid (42.0 mg, 0.08 mmol, 59% yield); LRMS m/z (APCI⁺) 474 [M+H]; 500 MHz ¹HNMR (CD₃OD) δ 7.89 (s, 1H); 7.73 (s, 2H); 7.37-7.29 (m, 3H); 7.23 (dd, J=6.6, 0.0 Hz, 2H); 5.49 (q, J=7.1 Hz, 1H); 4.51 (d, J=2.5 Hz, 1H); 3.64 (d, J=3.3 Hz, 1H); 3.53-3.50 (m, 1H); 3.03 (s, 3H); 2.90 (ddd, J=12.4, 4.2, 4.2 Hz, 1H); 2.18-2.14 (m, 1H); 2.08-2.03 (m, 1H); 1.89-1.85 (m, 2H); 1.59 (d, J=7.1 Hz, 3H).

### Example 231

### 3-(S)-Methylamino-2-(S)-phenyl-piperidine-1-carboxylic acid [1-(3,5-bis-trifluoromethyl-phenyl)-ethyl]-methyl-amide

Using example 227, the same procedure was followed as for example 222. The desired material was obatined as a viscous oil and converted directly to the mono-HCl salt using 440 uL 2N HCl/Et₂O and crystallized with isopropyl ether to give the product as a colorless solid (0.22 g, 0.42 mmol, 48% yield); LRMS m/z (APCI⁺) 488 [M+H]; 500 MHz ¹HNMR (CD₃OD) δ 7.89 (s, 1H); 7.81 (s, 2H); 7.43-7.37 (m, 5H); 5.30 (q, J=6.9 Hz, 1H); 4.96 (d, J=3.7 Hz, 1H); 3.70-3.67 (m, 1H); 3.55-3.49 (m, 1H); 3.26-3.23 (m, 1H); 2.82 (s, 3H); 2.55 (s, 3H); 2.17-2.23 (m, 1H); 2.17-2.11 (m, 1H); 1.92 (bs, 1H); 1.82-1.80 (m, 1H); 1.60 (d, J=7.1 Hz, 3H).

### Example 232

### 3-(S)-Methylamino-2-(S)-phenyl-piperidine-1-carboxylic acid [1-(3,5-bis-trifluoromethyl-phenyl)-ethyl]-methyl-amide (more polar)

Using example 228, the same procedure was followed as for example 222. The desired material was obtained as a viscous oil and converted directly to the mono-HCl salt using 170 uL of 2N HCl/Et₂O and crystallizing with isopropyl ether to obtain the product as a colorless solid (60.0 mg, 0.12 mmol, 34% yield); LRMS m/z (APCI⁺) 488 [M+H]; 500 MHz ¹HNMR (CD₃OD) δ 7.88 (s, 1H); 7.79 (s, 2H); 7.41-7.33 (m, 5H); 5.43 (q, J=7.1 Hz, 1H); 4.77 (d, J=2.9 Hz, 1H); 3.60 (bs, 1H); 3.52-3.49 (m, 1H); 3.06-3.03 (m, 1H); 2.97 (s, 3H); 2.44 (s, 3H); 2.27-2.26 (m, 1H); 2.09 (dddd, J=6.2, 6.2, 0.0, 0.0H, 1H); 1.85-1.84 (m, 2H); 1.60 (d, J=7.1 Hz, 3H).

### Example 233

### 3-(S)-Amino-2-(R)-phenyl-piperidine-1-carboxylic acid [1-(3,5-bis-trifluoromethyl-phenyl)-ethyl]-methyl-amide

Using intermediate 48, the same procedure was used as for example 222. The desired material was obtained as an oil and converted directly to the mono-HCl salt using 50 uL of 2N HCl/Et2O and crystallizing with isopropyl ether to give the product as a colorless solid (23.0 mg, 0.05 mmol, 48% yield); LRMS m/z (APCI⁺) 474 [M+H]; 500 MHz ¹HNMR (CD₃OD) δ 7.89 (s, 1H); 7.78 (s, 2H); 7.40-7.31 (m, 5H); 5.41 (q, J=6.8 Hz, 1H); 4.52 (d, J=6.2 Hz, 1H); 3.76 (bs, 1H); 3.28-3.25 (m, 1H); 3.13-3.09 (m, 1H); 2.76 (s, 3H); 2.16-2.12 (m, 1H); 1.91 (bs, 1H); 1.82-1.73 (m, 2H); 1.56 (d, J=7.1 Hz, 3H).

### Example 234

### 3-(R)-Methylamino-2-(S)-phenyl-piperidine-1-carboxylic acid [1-(3,5-bis-trifluoromethyl-phenyl)-ethyl]-methyl-amide

Using intermediate 50, the same procedure was followed as was used for example 222. The desired material was obtained as an oil and was directly converted to the mono-HCl salt using 120 uL 2N HCl/Et₂O and crystallizing with isopropyl ether to give the product as a colorless solid (65.0 mg, 0.12 mmol, 52% yield); LRMS m/z (APCI⁺) 488 [M+H]; ]; 500 MHz ¹HNMR (CD₃OD) δ 7.92 (s, 1H); 7.88 (s, 2H); 7.44-7.39 (m, 4H); 7.36-7.34 (m, 1H); 5.44 (q, J=6.9 Hz, 1H); 5.16 (d, J=3.7 Hz, 1H); 3.98-3.97 (m, 1H); 3.38-3.22 (m, 2H); 2.82 (s, 3H); 2.73 (s, 3H); 2.08-2.05 (m, 1H); 1.98-1.97 (m, 1H); 1.90-1.60 (m, 2H); 1.66 (d, J=7.1 Hz, 3H).

### Example 235

### 3-(R)-Methylamino-2-(S)-phenyl-piperidine-1-carboxylic acid [1-(3,5-bis-trifluoromethyl-phenyl)-ethyl]-methyl-amide

Using intermediate 51, the same procedure was followed as for example 222. The desired material was obtained as an oil and converted directly into the mono HCl salt using 75 uL of 2N HCl/Et₂O and crystallized with hexanes to give the product as a colorless solid (35.0 mg, 0.07 mmol, 43% yield); LRMS m/z (APCI⁺) 488 [M+H]; 500 MHz ¹HNMR (CD₃OD) δ 7.87 (s, 1H); 7.81 (s, 2H); 7.42-7.37 (m, 4H); 7.34-7.31 (m, 1H); 5.48 (q, J=7.1 Hz, 1H); 4.88 (d, 1H), 3.84 (bs, 1H); 3.30-3.25 (m, 2H); 2.86 (s, 3H); 2.73 (s, 3H); 2.17-2.16 (m, 1H); 1.86-1.80 (bm, 2H); 1.72 (bs, 1H); 1.61 (d, J=7.1 Hz, 3H).

### Example 236

### 3-(R)-Amino-2-(S)-o-tolyl-piperidine-1-carboxylic acid [1-(R)-(3,5-bis-trifluoromethyl-phenyl)-ethyl]-methyl-amide

Using intermediate 56, the same procedure was followed as for example 222. The desired material was obtained as a colorless oil (70.0 mg, 0.14 mmol, 85% yield); Rf 0.5 (10% MeOH/CH₂Cl₂ with 0.2% NH₄OH); LRMS m/z (APCI⁺) 488 [M+H].

### Example 237

### (1-(R)-{[1-(3,5-Bis-trifluoromethyl-phenyl)-ethyl]-methyl-carbamoyl}-2-(S)-o-tolyl-piperidin-3-(R)-yl)-methyl-carbamic acid tert-butyl ester

Intermediate 56 (0.368 g, 0.627 mmol) was dissolved in 6.5 mL of anhydrous THF and a 1.0 M tBuOK in THF was added to the solution dropwise and the resulting suspension allowed to stir for 10 minutes. MeI (0.444 g, 3.130 mmol) was then added and the reaction was stirred at rt for 88 h. The reaction was then concentrated under reduced pressure and repartitioned between CH₂Cl₂ and saturated aqueous NaHCO₃. The organics were extracted and dried over MgSO₄, filtered and concentrated under reduced pressure. Purification was accomplished through flash chromatography on a 40M Biotage silica gel column using 20% EtOAc/Hexanes collecting 18 mL fractions. Product containing fractions were combined and concentrated under reduced pressure to give the desired product as a colorless solid (53.0 mg, 0.09 mmol, 14% yield); Rf 0.3 (25% EtOAc/Toluene); LRMS m/z (APCI⁺) 602 [M+H].

### Example 238

### 3-(R)-Pyrrolidin-1-yl-2-(S)-o-tolyl-piperidine-1-carboxylic acid [1-(R)-(3,5-bis-trifluoromethyl-phenyl)-ethyl]-methyl-amide

Example 236 (58.0 mg, 0.1 mmol) was dissolved in 1.5 mL of DMF under N₂. Et₃N (46.0 mg, 0.5 mmol) was added, followed by 1,4-dibromobutane (56.1 mg, 0.3 mmol) and the resulting reaction mixture was heated to 65° C. for 24 h. The reaction was then cooled to rt and stirred for an additional 96 h. The reaction was then diluted with CH₂Cl₂ and washed once with saturated 10 mL NaHCO₃ aqueous solution, followed by a wash with H₂O 10 mL and the combined organics are then dried over MgSO₄, filtered and concentrated under reduced pressure. Purification was accomplished through flash chromatography on a 4 g Isco silica gel column eluting with 20% acetone/hexanes and collecting 8 mL fractions. Product containing fractions were combined and concentrated under reduced pressure to give the desired material as a colorless solid (40.0 mg, 0.1 mmol, 58% yield); Rf 0.2 (20% acetone/hexanes); LRMS m/z (APCI⁺) 542 [M+H].

### Example 239

### 3-(R)-Dimethylamino-2-(S)-o-tolyl-piperidine-1-carboxylic acid [1-(R)-(3,5-bis-trifluoromethyl-phenyl)-ethyl]-methyl-amide

Using intermediate 59, the same procedure was followed as for example 221. The desired material was obtained as a pale oil and directly converted to the mono-HCl salt using 2N HCl/Et₂O to give the product as a colorless solid (0.165 g, 0.299 mmol, 91% yield); LRMS m/z (APCI⁺) 515 {M+H]; 500 MHz ¹HNMR (CD₃OD) δ 7.87 (s, 1H); 7.67 (s, 2H); 7.48 (dd, J=7.9, 0.0 Hz, 1H); 7.26-7.25 (m, 2H); 7.22-7.18 (m, 1H); 5.38 (q, J=6.9 Hz, 1H); 4.72 (d, J=8.7 Hz, 1H); 3.90 (bs, 1H); 3.27 (bs, 1H); 3.00 (ddd, J=13.7, 3.7, 3.7 Hz, 1H); 2.87 (s, 3H); 2.79 (s, 3H); 2.68 (s, 3H); 2.50 (s, 3H); 2.34 (ddd, J=9.1, 4.6, 4.6 Hz, 1H); 2.03-1.85 (m, 3H); 1.44 (d, J=7.1 Hz, 3H).

### Example 240

### (1-(R)-{[1-(3,5-Bis-trifluoromethyl-phenyl)ethyl]-methyl-carbamoyl}-2-(S)-o-tolyl-pyrrolidin-3-(R)-yl)-carbamic acid tert-butyl ester

The known benzylamine [1-(R)-(3,5-bistrifluoromethyl-phenyl)-ethyl]-methyl-amine [WO 01/25219AZ] (1.13 g, 4.17 mmol) was dissolved in 40 mL of anhydrous DCE and 2.3 mL of Et₃N (1.69 g, 16.90 mmol) was added. Triphosgene (0.41 g, 1.38 mmol) was separately dissolved in 5 mL of DCE and added to the reaction dropwise over 10 minutes. The resulting solution was stirred at rt for 1½ h. Intermediate 67 (0.86 g, 3.13 mmol) was then separately dissolved in 10 mL DCE and added to the reaction. The reaction solution was then heated in an oil bath at 55° C. for 1 h. The reaction was then cooled and quenched with saturated NaHCO₃ aqueous solution and extracted with CH₂Cl₂ (3×20 mL). Combined organics were then dried over MgSO₄, filtered and concentrated under reduced pressure. Purification was accomplished through flash chromatography on a 35 g Isco silica gel column eluting with 25% EtOAc/Hexanes and collecting 18 mL fractions. Product containing fractions were combined and concentrated under reduced pressure to give the desired material as a colorless solid (1.69 g, 2.95 mmol, 71% yield); Rf 0.4 (40% EtOAc/Hexanes); LRMS m/z (APCI⁺) 574/474 [M+H]; 500 MHz ¹HNMR (CDCl₃) δ 7.68 (s, 1H); 7.52 (s, 2H); 7.17-7.02 (m, 4H); 5.26 (q, J=6.9 Hz, 1H); 5.13 (d, J=6.2 Hz, 1H); 4.99 (bs, 1H); 4.01-3.97 (m, 1H); 3.86-3.78 (m, 2H); 2.49 (s, 3H); 2.28 (bs, 3H); 2.13 (ddd, J=19.9, 12.9, 7.1 Hz, 1H); 1.86 (bs, 1H); 1.47 (d, J=7.1 Hz, 3H); 1.36 (s, 9H).

### Example 241

### 3-(R)-Amino-2-(S)-o-tolyl-pyrrolidine-1-(R)-carboxylic acid [1-(3,5-bis-trifluoromethyl-phenyl)-ethyl]-methyl-amide

Using intermediate 68, the same procedure was followed as for example 222. The desired material was obtained as a colorless oil (15.0 mg, 0.03 mmol, 22% yield); Rf 0.3 (5% MeOH/CH₂Cl₂ with 0.2% NH₄OH); LRMS m/z (APCI⁺) 474 [M+H]; 500 MHz ¹HNMR (CD₃OD) δ 7.83 (s, 1H); 7.70 (s, 2H); 7.26 (dd, J=7.1, 0.0 Hz, 1H); 7.17-7.08 (m, 3H); 5.26 (q, J=7.2 Hz, 1H); 4.92 (d, J=6.2 Hz, 1H); 3.91 (ddd, J=10.0, 7.1, 7.1 Hz, 1H); 3.74-3.69 (m, 1H); 3.38-3.34 (m, 1H), 2.52 (s, 3H); 2.43 (s, 3H); 2.22-2.15 (m, 1H); 1.85 (dddd, J=12.0, 7.9, 7.9, 7.9 Hz, 1H); 1.60 (d, J=7.1 Hz, 3H).

### Example 242

### 3-(R)-Methylamino-2-(S)-o-tolyl-pyrrolidine-1-carboxylic acid [1-(R)-(3,5-bis-trifluoromethyl-phenyl)-ethyl]-methyl-amide

Using intermediate 69, the same procedure was followed as example 222. The desired material was obtained as a viscous oil (75.0 mg, 0.15 mmol, 81% yield); Rf 0.3 (5% MeOH/CH₂Cl₂ with 0.2% NH₄OH); LRMS m/z (APCI⁺) 488 [M+H]; 500 MHz ¹HNMR (CDCl₃) δ 7.73 (s, 1H); 7.56 (s, 2H); 7.23-7.07 (m, 4H); 5.28 (q, J=6.8 Hz, 1H); 4.98 (d, J=4.6 Hz, 1H); 3.81-3.73 (m, 2H); 3.06 (dddd, J=5.4, 5.4, 5.4, 0.0 Hz, 1H); 2.52 (s, 3H); 2.43 (s, 3H); 2.31 (s, 3H); 2.17-2.11 (m, 1H); 1.83-1.77 (m, 2H); 1.51 (d, J=6.6 Hz, 3H).

### Experimental Procedure for Examples 243-264 (Table 5)

The compounds listed in Table 5 were prepared using intermediate 24. The amines (for sidechain Z) (0.20 mmol, 4.0 eq) were pre-weighed in 1 dram, septa capped vials. The aldehyde (0.05 mmol, 1.0 eq) was dissolved in anhydrous THF and added to the reaction vials in 0.5 mL portions. The resulting solutions were stirred at rt for 16 h. To each reaction vial was then added Na(OAc)₃BH (30.00 mg, 2.5 eq) and the vials were shaken an additional 5 h. The reactions were then quenched by adding 1N NaOH (0.75 mL) and EtOAc (1.75 mL). The organics were separated and loaded onto an equilibrated SCX SPE (conditioned with 6 mL MeOH, CH₂Cl₂ and EtOAc). The desired products were then eluted off using 1N TEA in MeOH (5 mL). These solutions were collected in tared vials and dried under a N₂ stream. Purifications were accomplished by HPLC separation on a Waters Symmetry C¹⁸ column (5 mm, 3.9×150 mm) with a 1.0 mL/min flow rate eluting with a gradient system of 100%, 80%, 0% (0.1% TFA in H₂O/CH₃CN) injecting each sample in 2 mL of solvent. Isolated and tested as the TFA salts.

TABLE 5

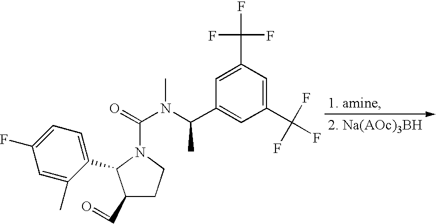

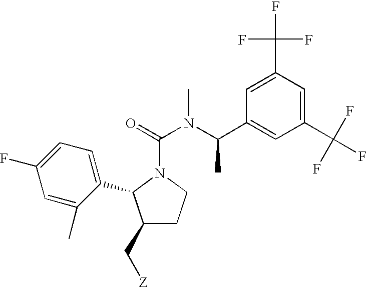

| Example | Z | m/z [M + H] |
|---|---|---|
| 243 | 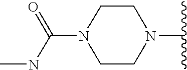 | 631.28 |
| 244 | 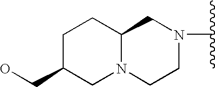 | 658.31 |
| 245 | 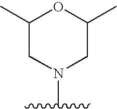 | 603.27 |
| 246 | 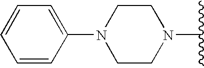 | 650.29 |
| 247 | 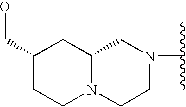 | 658.31 |
| 248 | 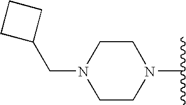 | 642.32 |
| 249 | 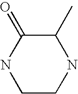 | 602.25 |
| 250 | 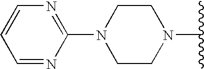 | 652.28 |
| 251 | 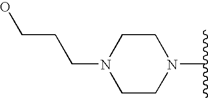 | 632.3 |
| 252 | 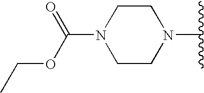 | 646.28 |
| 253 | 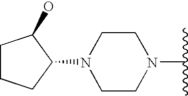 | 658.31 |
| 254 | 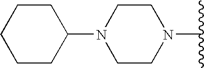 | 656.33 |
| 255 | 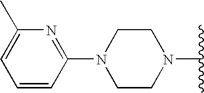 | 655.3 |
| 256 | 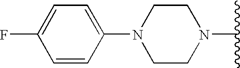 | 668.28 |
| 257 | 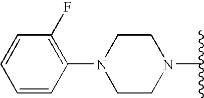 | 668.28 |
| 258 | 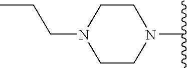 | 616.3 |
| 259 | 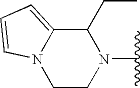 | 638.29 |
| 260 | 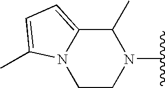 | 638.29 |
| 261 | 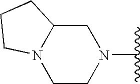 | 614.29 |
| 262 | 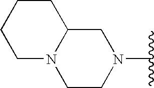 | 628.3 |
| 263 | 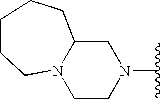 | 642.32 |
| 264 | 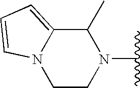 | 624.27 |
| 264a | 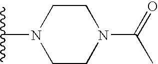 | 617 |
| 264b | 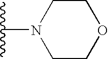 | 576 |

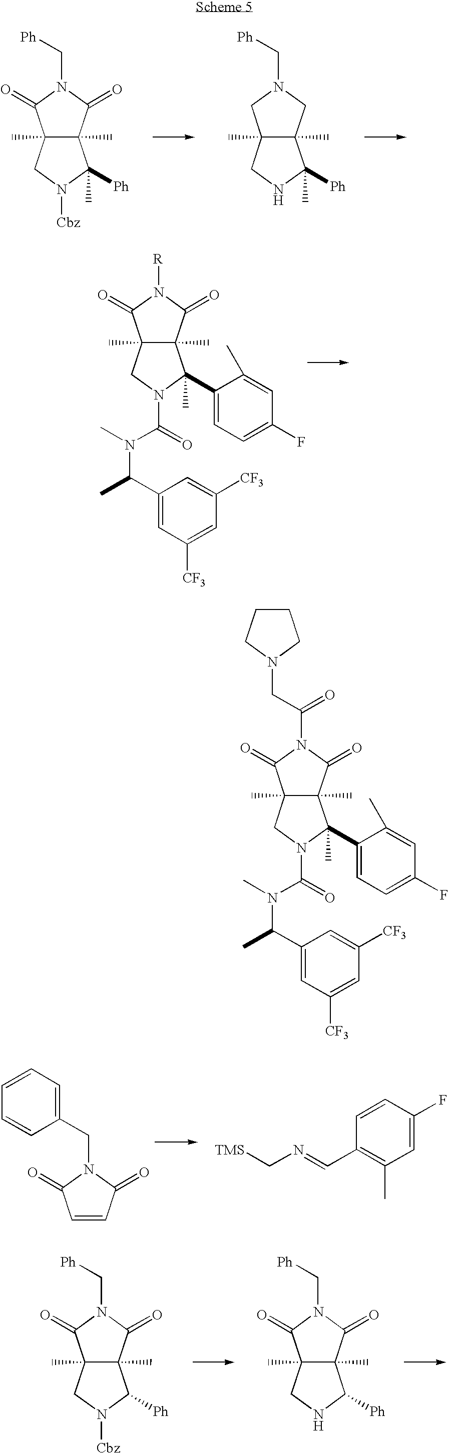

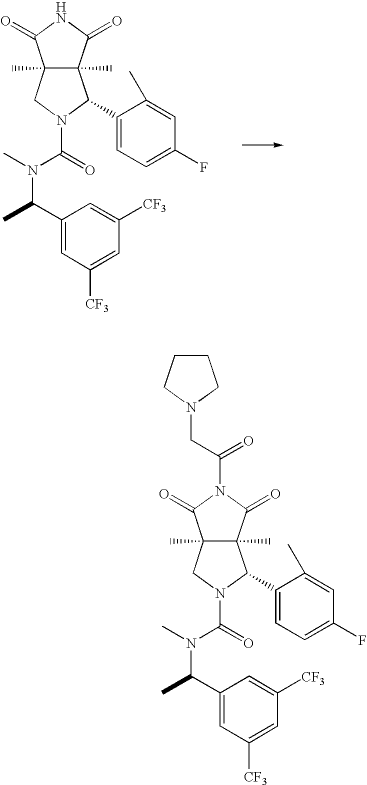

Other embodiments of the invention may be prepared according to Scheme 5: A substituted benzaldehyde, phenethylaldehyde or a substituted phenylacetaldehyde in a solvent such as toluene, benzene, cyclohexane or similar inert solvent but preferably toluene was treated with trimethylsilylmethylamine or other alkylsusbstitutedsilylmethyl amine with or without a desiccant such as 3 angstrom molecular sieves, magnesium sulfate, sodium sulfate or sodium hydroxide. The reaction was stirred under inert atmosphere protected from moisture for 16-48 hours at ambient temperature or up to the reflux point of the solvent with collection of water using a Dean Stark type apparatus without the use of a dessicant. The mixture was filtered through celite and the filtrate was evaporated in vacuo to afford the intermediate. The most preferred conditions with a benzaldehyde use 3 A molecular sieves in toluene for 24 hours at ambient temperature.

In a suitable anhydrous inert solvent such as THF ether, dioxane, toluene or dichloroethane was charged most conveniently carbobenzyloxycarbonyl chloride (CBz-Cl) but also other acid chlorides or chloroformates such as t-butyloxychloroformate, methyl or ethylchloroformate, trichloroethylchloroformate and fluorenylmethyl chloroformate followed by N-benzylmaleimide or other N-protected forms of maleimide compatible with the protection of the imide nitrogen of the ring. The reaction mixture was heated from ambient temperature to the reflux point of the solvent with the most preferred temperature being 45° C. A solution of the trimethylsilanylmethyl benzylidene intermediate prepared from above was added dropwise via syringe pump or other calibrated device over a period of 30 minutes to two hours with one hour being most preferred. The reaction was stirred for one to 16 hours with two additional hours being preferred and then cooled to ambient temperature. The product was obtained after concentration in vacuo and chromatography on silica gel eluting with a mixture of ethyl acetate/hexanes or other suitable solvent or mixture of solvents to yield two separated diastereomeric products. Each of these separated diastereomers were taken through the following series of steps together or separately.

One or both of the product diastereomers prepared above was dissolved in an anhydrous solvent such as ether, dioxane, dimethoxyethane or most preferably THF. To this solution was added most preferably sodium borohydride or other suitable reducing agent such as borane, borane-THF or borane-dimethylsulfide followed by cooling the reaction mixture to 0° C. If sodium borohydride is used then boron trifluoride etherate was added dropwise over 0.5 to 2 minutes with one minute being preferred and the reaction was warmed to ambient temperature followed by heating to the reflux point of the solvent for 2-12 hours preferably 4 hours. The reaction mixture can be quenched in a variety of ways. In one, the mixture was carefully treated with excess piperazine in a portionwise fashion followed by addition of water. Alternatively one may use dimethyl amine or other secondary amines in a solution of water or alcohol. Alternatively, one may simply use water, methanol or ethanol to quench. Additionally, one may employ an olefin such as cyclohexene and palladium on carbon or palladium hydroxide as part of the quench system. However, for the quench conducted with piperazine or other amine or alcohols, the mixture was heated the reflux point of the solvent for 12-24 hours with 16 hours being preferred. After cooling to ambient temperature. The mixture was diluted with water and extracted with an organic solvent such as ethyl acetate. The organic phase was dried and evaporated to yield a clear yellow oil.

The material prepared above can be mono deprotected on nitrogen by one of several methods. The substrate in methanol (ethanol or other suitable alcohol) can be treated with ammonium formate and 10% palladium on carbon. The mixture was heated to reflux for 0.5-12 hours preferably 30 minutes. The reaction mixture was filtered through celite and the solvent was evaporated in vacuo. Alternatively, the substrate was hydrogenated under 1-10 atmospheres of hydrogen and a suitable catalyst such as palladium on carbon or palladium hydroxide in a suitable solvent such as methanol, ethanol or the like. Alternatively, the preferred method utilizes 48% HBr in acetic acid stirring for 0.5-10 hours preferably 30 minutes. The dark mixture was treated with diethyl ether or other suitable organic solvent whereupon a precipitate of the desired product-HBr salt was formed and collected.

A suitably substituted benzyl amine or phenethylamine in a solvent such as toluene, dichloroethane, dioxane, THF, ether, methylene chloride but preferably toluene was treated with a base such as triethylamine diisopropylethylamine, N-methylpiperidine, N-methylmorpholine but preferably triethylamine. To this solution was added phosgene gas, 20% phosgene in toluene or trichloromethyl chloroformate but preferably 20% phosgene in toluene followed by stirring at ambient temperature for 1-10 hours but preferably 4 hours. The mixture was then treated with a acylation catalyst such as pyridine but preferably DMAP and a base such as triethylamine diisopropylethylamine, N-methylpiperidine, N-methylmorpholine but preferably triethylamine followed by addition of the mono-deprotected amine substrate prepared above. The reaction mixture was heated to 100° C. or the reflux point of the solvent for 1-24 hours but preferably 16 hours and then allowed to cool to ambient temperature. The solvent was evaporated in vacuo and the residue was partitioned between a suitable organic and aqueous base solution. Typically, methylene chloride or ethyl acetate is used in combination with a saturated solution of sodium bicarbonate. The organic layer was washed with water, brine and then dried and evaporated in vacuo. The residue was chromatographed on silica gel eluting with THF-petroleum ether or other suitable solvents such as mixtures of ether hexane or ethyl acetate hexane to afford the desired products.

The starting material from above was dissolved in methanol (ethanol or other suitable alcohol) and treated with ammonium formate and 10% palladium on carbon. The mixture was heated under reflux for 0.5-12 hours preferably 30 minutes. The reaction mixture was filtered through celite and the solvent was evaporated in vacuo. Alternatively, the substrate was hydrogenation under 1-10 atmospheres of hydrogen and a suitable catalyst such as palladium on carbon or palladium hydroxide in a suitable solvent such as methanol, ethanol, water, ethyl acetate, acetic acid or the like. The reaction mixture was filtered through celite and the solvent was evaporated in vacuo. The residue was taken up in methylene chloride washed with saturated aqueous sodium bicarbonate solution and then washed with brine, dried over sodium sulfate and evaporated in vacuo to afford a clear oil.

To a flame dried round bottomed flask with nitrogen inlet and magnetic stir bar was added the product from above and a suitable amino acid in a solvent such as methylene chloride, THF, dioxane, ethyl acetate, dichloroethane, ether, dimethoxyethane toluene but preferably methylene chloride. The mixture was treated with a base such as triethylamine, N-methylmorpholine, 1-methyl piperidine but preferably diisopropylethylamine (Hunig's base) and a suitable coupling reagent chosen from but not limited to BOP-Cl, dicyclohhexylcarbodiimide, 1-(3-dimethylaminopropyl)-3-ethylcarbodiimide, HBTU, TBTU, isobutyl chloroformate but preferably the “BOP reagent CAS [56602-33-6]”. The reaction mixture was stirred at ambient temperature for 10-48 hours but preferably 16 hours. The reaction mixture was partitioned between water and ethyl acetate and the organic phase was washed several times with water and then dried over sodium sulfate and evaporated in vacuo. The residue was triturated with ether to afford a yellow solid.

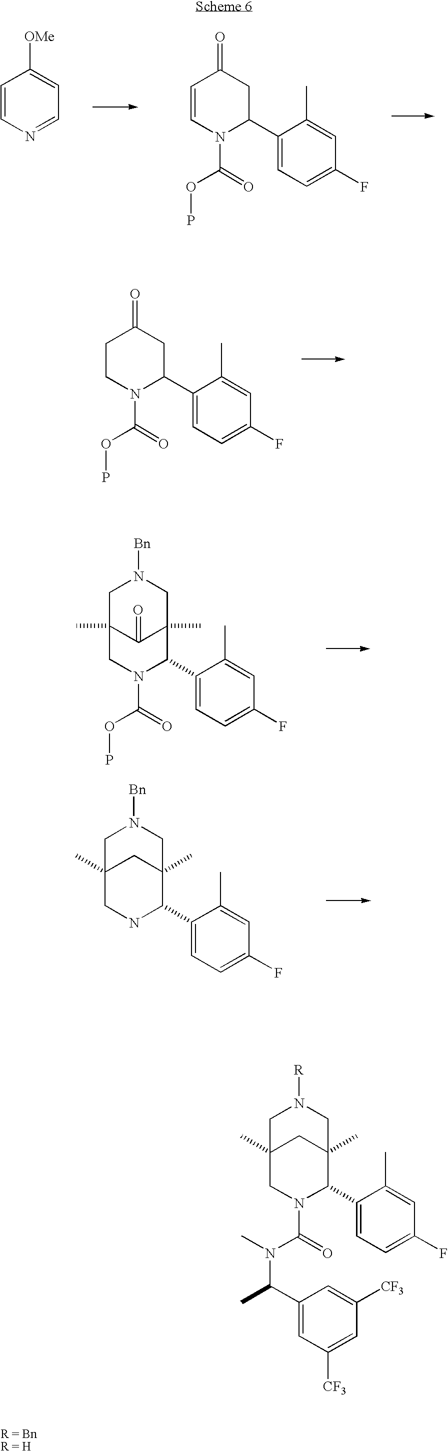

In Scheme 6 above, a solution of a 6-alkoxypyridine such as 6-benzyloxypyridine or 6-ethoxypyridine but preferably 6-methoxypyridine in an anhydrous solvent such as THF, methylene chloride, dichloroethane, ether but preferably THF was treated dropwise with a suitably substituted aromatic or benzylic grignard reagent in THF, toluene or diethyl ether over 1-60 minutes preferably 10 minutes. The solution was allowed to stir for 1-5 hours at ambient temperature, preferably 1 hour and then cooled to between 0 and −40° C. preferably −23° C. A suitable chloroformate such as phenyl, methyl, ethyl, trichloroethyl, fluorenylmethyl, t-butyloxy, vinyl or preferably benzyl chloroformate was added drop wise and the reaction mixture was stirred at the same temperature for 1-10 hours preferably 1 hour. The mixture was quenched in aqueous acid such as sulfuric or preferably HCl and stirred for 16-24 hours at room temperature, preferably 16 hours. The organic solvent was removed using a rotary evaporator and replaced with an equal volume of ethyl acetate. The organic phase was washed with saturated carbonate solution and then brine. The organic phase was dried over sodium sulfate and the volume was reduced using a rotary evaporator. Hexanes was added to afford a white precipitate. Filtration followed by washing with hexane afforded a pale yellow solid.

A solution of the product from above in acetic acid was treated with zinc metal in any form but preferably as the “dust”. The reaction mixture was stirred for 2-48 hours but preferably 20 hours at ambient temperature. The reaction mixture was filtered and the solid mass was washed with an organic solvent such as but not limited to ethyl acetate. The filtrate was evaporated in vacuo and the residue was diluted with water and then basified by carefully adding a saturated aqueous solution of potassium carbonate. The mixture was extracted with ethyl acetate and the organic phase was washed with brine and then dried over sodium sulfate and evaporated in vacuo to afford a yellow oil. Alternatively, when benzyl was not used for protection, The starting material from above was dissolved in methanol (ethanol or other suitable alcohol) and treated with ammonium formate and 10% palladium on carbon. The mixture was heated under reflux for 0.5-12 hours preferably 30 minutes. The reaction mixture was filtered through celite and the solvent was evaporated in vacuo. Alternatively, the substrate was hydrogenation under 1-10 atmospheres of hydrogen and a suitable catalyst such as palladium on carbon or palladium hydroxide in a suitable solvent such as methanol, ethanol, water, ethyl acetate, acetic acid or the like. The reaction mixture was filtered through celite and the solvent was evaporated in vacuo. The residue was taken up in methylene chloride washed with saturated aqueous sodium bicarbonate solution and then washed with brine, dried over sodium sulfate and evaporated in vacuo to afford a clear oil.

A solution containing a primary amine such as methyl amine or benzylamine, together with formalin solution or paraformaldehyde, acetic acid, aqueous hydrochloric or sulfuric acid and methanol or ethanol was heated up to the reflux point of the solvent but preferably 65° C. for 1-6 hours but preferably 1 hour. A second solution consisting of the product from the previous experiment and acetic acid in methanol or ethanol was then added drop wise and the resultant mixture was heated under reflux for 10-24 hours but preferably 16 hours. The reaction mixture was cooled to room temperature and the solvent was removed in vacuo. The residue was diluted with water and then basified by carefully adding a saturated aqueous solution of potassium carbonate. The mixture was extracted with an organic solvent ethyl acetate and the organic phase was washed with water, brine and then dried over sodium sulfate and evaporated in vacuo. The residue was chromatographed on silica gel eluting with a mixture of ethyl acetate in hexane to afford a white foam.

In a single neck round bottom flask fitted with a condenser and nitrogen inlet was combined tosylhydrazine or hydrazine and the product from above in methanol or ethanol or ethylene glycol. Preferably tosylhydrazine and methanol were used. The reaction mixture was heated to 50-100° C. preferably 65° C. for 1-5 hours but preferably 2 hours and then allowed to cool to room temperature and was stirred for 10 to 24 hours but preferably 16 hours. When hydrazine is used the reaction is preferentially carried out in ethylene glycol with sodium hydroxide at a temperature near 200° C. The solution was evaporated in vacuo to afford a white solid that was used without purification. To a flame dried round bottomed flask was charged the tosylhydrazone prepared above and dichloroethane, methylene chloride or preferably chloroform. This was followed by addition of catecholborane, borane, diacetoxyborane or other borane derived reducing agents and the reaction mixture was stirred for 1-6 hours but preferably 2 hours at room temperature. The mixture was treated with sodium acetate whereupon gas evolution was observed. The mixture was stirred for 10-24 hours but preferably 16 hours at room temperature. The solvent was removed in vacuo and the residue was treated with methanol and the mixture was refluxed for 1 hour. The reaction mixture was cooled to room temperature and evaporated in vacuo. The residue was taken up in methylene chloride or dichloroethane, filtered through celite, and the filtrate was evaporated in vacuo. The residue was chromatographed on silica gel (elution with ethyl acetate in hexane or other mixtures of organic solvents such as ether/hexane) to afford the desired product.

Deprotection of the phenylcarbamate: To a solution of lithium hydroxide, sodium hydroxide or preferably potassium hydroxide in 10 ml of ethanol or other alcohol was added the phenylcarbamate from above. The reaction was heated to reflux for 10-24 hours but preferably 16 hours. The reaction mixture was allowed to cool to room temperature and was then concentrated in vacuo. The residue was diluted with water and extracted with methylene chloride or ethyl acetate or other suitable organic solvent. The organic phase was washed with brine and dried over sodium sulfate and evaporated. Chromatography on silica gel (elution with 15% methanol in methylene chloride with 1% added ammonium hydroxide) afforded a colorless oil.

Deprotection of the benzyl carbamate: To benzyl ester at 0° C. was added a solution of 30% HBr preferably in acetic acid but equally effective is an aqueous solution. The mixture was stirred for 2-10 hours but preferably 2 hours and was then diluted with diethylether or diisopropyl ether. The mixture was concentrated in vacuo and the residue was taken up in water. The mixture was made basic to pH 10 with 2N sodium hydroxide and was then extracted with methylene chloride or other suitable organic solvent. The organic phase was washed with water, brine and then dried over sodium sulfate and evaporated in vacuo. Chromatography on silica gel (elution with 3% methanol in methylene chloride with 1% added ammonium hydroxide) afforded a pale yellow oil. Alternatively, the starting material from above was dissolved in methanol (ethanol or other suitable alcohol) and treated with ammonium formate and 10% palladium on carbon. The mixture was heated under reflux for 0.5-12 hours preferably 30 minutes. The reaction mixture was filtered through celite and the solvent was evaporated in vacuo. Alternatively, the substrate was hydrogenation under 1-10 atmospheres of hydrogen and a suitable catalyst such as palladium on carbon or palladium hydroxide in a suitable solvent such as methanol, ethanol, water, ethyl acetate, acetic acid or the like. The reaction mixture was filtered through celite and the solvent was evaporated in vacuo. The residue was taken up in methylene chloride washed with saturated aqueous sodium bicarbonate solution and then washed with brine, dried over sodium sulfate and evaporated in vacuo to afford a clear oil.

A suitably substituted benzyl amine or phenethylamine in a solvent such as toluene, dichloroethane, dioxane, THF, ether, methylene chloride but preferably toluene was treated with a base such as triethylamine diisopropylethylamine, N-methylpiperidine, N-methylmorpholine but preferably triethylamine. To this solution was added phosgene gas, 20% phosgene in toluene or trichloromethyl chloroformate but preferably 20% phosgene in toluene followed by stirring at ambient temperature for 1-10 hours but preferably 4 hours. The mixture was then treated with a acylation catalyst such as pyridine, polystyrene dimethyl aminopyridine (PS-DMAP) but preferably dimethyl aminopyridine (DMAP) and a base such as triethylamine diisopropylethylamine, N-methylpiperidine, N-methylmorpholine but preferably triethylamine followed by addition of the mono-deprotected amine substrate prepared above. The reaction mixture was heated to 100° C. or the reflux point of the solvent for 1-24 hours but preferably 16 hours and then allowed to cool to ambient temperature. The solvent was evaporated in vacuo and the residue was partitioned between a suitable organic and aqueous base solution. Typically, methylene chloride or ethyl acetate is used in combination with a saturated solution of sodium bicarbonate. The organic layer was washed with water, brine and then dried and evaporated in vacuo. The residue was chromatographed on silica gel eluting with 10-30% ethyl acetate in hexanes to afford a pale yellow solid alternatively THF-petroleum ether or other suitable solvents such as mixtures of ether hexane or ethyl acetate hexane to afford the desired products.

The product from above was dissolved in methanol (ethanol or other suitable alcohol) and treated with ammonium formate and 10% palladium on carbon. The mixture was heated under reflux for 0.5-12 hours preferably 30 minutes. The reaction mixture was filtered through celite and the solvent was evaporated in vacuo. Alternatively, the substrate was hydrogenation under 1-10 atmospheres of hydrogen and a suitable catalyst such as palladium on carbon or palladium hydroxide in a suitable solvent such as methanol, ethanol, water, ethyl acetate, acetic acid or the like. The reaction mixture was filtered through celite and the solvent was evaporated in vacuo. The residue was taken up in methylene chloride washed with saturated aqueous sodium bicarbonate solution and then washed with brine, dried over sodium sulfate and evaporated in vacuo to afford a clear oil

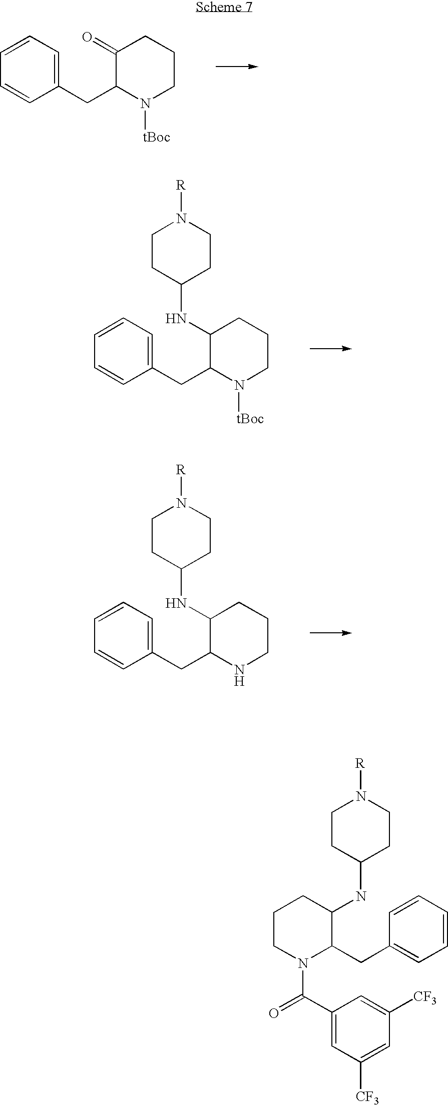

In Scheme 7 above, one of a variety of possible N-substituted-piperidin-4-ones was dissolved in an alcohol such as ethanol and treated with hydroxylamine hydrochloride and pyridine. The reaction mixture was heated to 70° C. for 0.5-3 hours but preferably 1.5 hr. The solvent was removed and the residue was treated with water and cooled to 0° C. The resulting slurry was filtered and dried under vacuum to afford an oxime as a white solid. The oxime in a Parr bottle was dissolved in of ethanol and treated with 0.2 gm of Raney nickel which had been washed several times with water. The reaction was placed in to Parr apparatus and hydrogenated under 1-200 psi but preferably 50 psi hydrogen pressure for 10-24 hours but preferrably 16 hours. The mixture was carefully filtered and the filtrate was evaporated in vacuo to afford of the desired amine as a solid.

A solution of N-substituted-piperidin-4-one and 2-Benzyl-3-oxopiperidine-1-carboxylic acid tert-butyl ester Prepared according to the procedure of Brubaker and Colley: J. Medicinal Chem. 1986, 29, 1528-1531 preferrably in toluene but also in benzene or cyclohexane was heated under reflux over a Dean-Stark water separator for 10-24 hours but usually 18 hours. The reaction mixture was cooled to room temperature and was treated with a reducing agent such as sodium cyanoborohydride, triethylsilane, tetrabutylammonium cyanoborohydride or preferrrably sodium triacetoxyborohydride. The reaction mixture was then stirred for 10-36 hours or preferably for 20 hours at ambient temperature. The reaction mixture was quenched by adding saturated bicarbonate solution and then extracted with methylene chloride or other suitable organic solvent such as dichloroethane. The organic phase was washed with water and saturated brine. The organic phase was then dried over sodium sulfate and evaporated in vacuo to afford a yellow oil. The oil was chromatographed on silica gel (elution with 5% methanol in methylene chloride with 1 ml/100 ml of conc ammonium hydroxide) to afford an oil.

A solution of the product from above in methylene chloride, toluene or preferably dichloroethane was treated preferably with trifluoroacetic acid or other acids such as acetic, hydrochloric and hydrobromic then heated under reflux for 1-6 hours but usually 2 hr. The reaction mixture was cooled to ambient temperature and evaporated in vacuo. The residue was diluted with water and basified by adding 2 N sodium hydroxide solution to pH 9.0 and then extracted with methylene chloride or other organic solvent such as ethyl acetate. The organic phase was washed with water and saturated brine. The organic phase was then dried over sodium sulfate and evaporated in vacuo to afford an oil.

To a solution of the product from above in methylene chloride, toluene or preferably dichloroethane was added an acylation catalyst such as pyridine, PS-DMAP but preferably DMAP and a base such as triethylamine diisopropylethylamine, N-methylpiperidine, N-methylmorpholine but preferably triethylamine. The solution was treated with a substituted benzoyl chloride and stirred at room temperature preferably for 20 hours. The reaction mixture was diluted with methylene chloride. The organic phase was washed with water and saturated brine. The organic phase was then dried over sodium sulfate and evaporated in vacuo. The residue was chromatographed on silica gel (elution with 5% methanol in methylene chloride with 1 ml/100 ml of conc ammonium hydroxide) to afford a pale yellow foam. This compound is a mixture of cis and trans isomers which was separated by chiral chromatography into a mixture of cis and trans enantiomeric pairs. For instance Chiralcel OD (4.6 mm×25 cm) 80/20 Heptane/EtOH

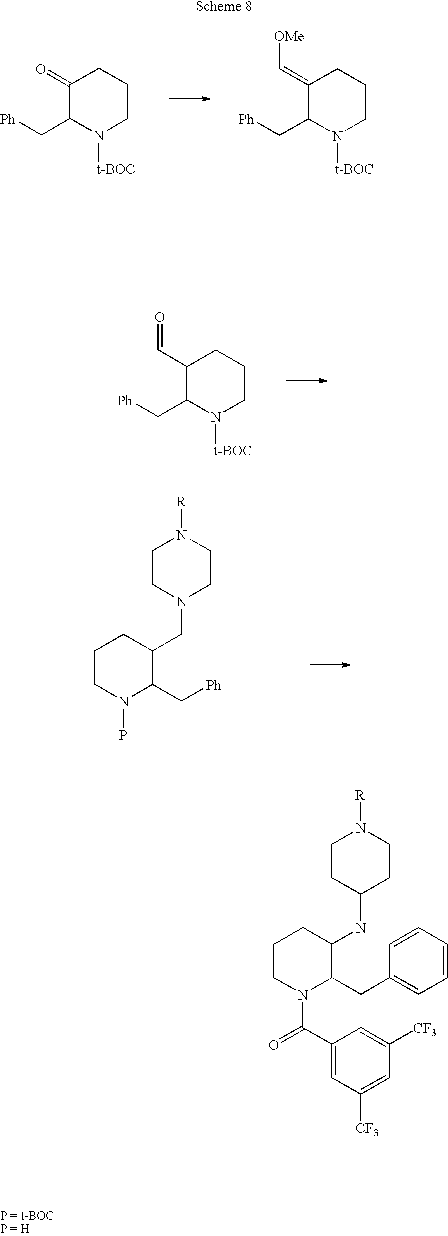

In Scheme 8 above, to a stirred solution of methoxymethytriphenylphosphonium chloride in anhydrous diethyl ether or preferrably THF at −78° C. was added a solution of a base such as potassium t-butoxide, sodium hydride, sodium or lithium hexamethyldisilazide or preferrably lithium diisopropylamide (prepared at −78° C. n butyl lithium and diisopropyl amine) in dimethoxyethane, diethylether or preferably in anhydrous THF. The reaction mixture was stirred for 30-120 minutes but usually 40 min at a temperature from 0-−100° C. preferably −78° C. and then a solution of 2-Benzyl-3-oxopiperidine-1-carboxylic acid tert-butyl ester prepared according to the procedure of Brubaker and Colley: J. Medicinal Chem. 1986, 29, 1528-1531 in anhydrous ether or preferably THF was added. The mixture was stirred for 1-60 minutes but preferrably 10 min at 0-−100° C. preferably −78° C. and then allowed to warm to room temperature and stirred for 1-5 hours but usually 1.5 hours. The reaction mixture was then heated under reflux for 10-24 hours preferably 16 hours. The reaction mixture was cooled and quenched by adding saturated brine solution and then extracted with methylene chloride or other organic solvent such as ethyl acetate. The organic phase was washed with saturated sodium bicarbonate solution followed by water and saturated brine. The organic phase was then dried over sodium sulfate and evaporated in vacuo to afford a yellow oil. The oil was chromatographed on silica gel (elution with a mixture of ethyl acetate in hexane) to afford an oil.

To a room temperature solution of acid such as hydrochloric, sulfuric, trifluoroacetic or preferably 3M aqueous HCl and THF or other water miscible solvent was added the product from above and stirred for 5-24 hours but preferrably 6 hours. The reaction mixture was extracted with methylene chloride or other organic solvent such as ethyl acetate. The organic phase was washed with saturated sodium bicarbonate solution followed by saturated brine and then dried over sodium sulfate and evaporated in vacuo to afford a yellow oil.

A solution of aldehyde from above and monosubstituted piperazine in dichloroethane was stirred at ambient temperature under nitrogen for 15 minutes before adding a reducing agent such as sodium cyanoborohydride, triethylsilane, tetrabutylammonium cyanoborohydride or preferrrably sodium triacetoxyborohydride. The reaction mixture was then stirred for 10-36 hours or preferably for 20 hours at ambient temperature. The reaction mixture was quenched by adding saturated bicarbonate solution and then extracted with methylene chloride or other suitable organic solvent such as dichloroethane. The organic phase was washed with water and saturated brine. The organic phase was then dried over sodium sulfate and evaporated in vacuo to afford a yellow oil. The oil was chromatographed on silica gel (elution with 5% methanol in methylene chloride with 1 ml/100 ml of conc ammonium hydroxide) to afford an oil.

A solution of the product from above in methylene chloride, toluene or preferably dichloroethane was treated preferably with trifluoroacetic acid or other acids such as acetic, hydrochloric and hydrobromic then heated under reflux for 1-6 hours but usually 2 hr. The reaction mixture was cooled to ambient temperature and evaporated in vacuo. The residue was diluted with water and basified by adding 2 N sodium hydroxide solution to pH 9.0 and then extracted with methylene chloride or other organic solvent such as ethyl acetate. The organic phase was washed with water and saturated brine. The organic phase was then dried over sodium sulfate and evaporated in vacuo to afford an oil.

To a solution of the product from above in methylene chloride, toluene or preferably dichloroethane was added an acylation catalyst such as pyridine, PS-DMAP but preferably DMAP and a base such as triethylamine diisopropylethylamine, N-methylpiperidine, N-methylmorpholine but preferably triethylamine. The solution was treated with a substituted benzoyl chloride and stirred at room temperature preferably for 20 hours. The reaction mixture was diluted with methylene chloride. The organic phase was washed with water and saturated brine. The organic phase was then dried over sodium sulfate and evaporated in vacuo. The residue was chromatographed on silica gel (elution with 5% methanol in methylene chloride with 1 ml/100 ml of conc ammonium hydroxide) to afford a pale yellow foam. This compound is a mixture of cis and trans isomers which was separated by chiral chromatography into a mixture of cis and trans enantiomeric pairs. For instance Chiralcel OD (4.6 mm×25 cm) 80/20 Heptane/EtOH

Specific examples are as follows:

### Example 265

### (4-Fluoro-2-methyl-benzylidene)-trimethylsilanylmethylamine

0.56 gm (4 mmol) 4-Fluoro-2-methyl-benzaldehyde in 10 ml of toluene was treated with 0.566 ml (4 mmol) trimethylsilylmethylamine followed by a spatula tip (unmeasured amount) of 3 angstrom molecular sieves. The reaction was stirred under nitrogen for 24 hours at room temperature. The mixture was filtered through celite and the filtrate was evaporated in vacuo to afford 0.770 gm (86%). The product was used directly in the next step. Mass spectrum APCI m/z=224 (p+1)

### Example 266

### (2-methyl-benzylidene)-trimethylsilanylmethyl-amine

Using a procedure similar to Example 265; Mass spectrum APCI m/z=206 (p+1)

### Example 267

### Benzylidene-trimethylsilanylmethyl-amine

Using a procedure similar to Example 265; Mass spectrum APCI m/z=192 (p+1)

### Example 268

### 5-Benzyl-1-(4-fluoro-2-methyl-phenyl)-4,6-dioxo-hexahydro-pyrrolo[3,4-c]pyrrole-2-carboxylic acid benzyl ester

[ref. Chem. Pharm. Bull., 31 (11)3939-3945 (1983)] To a flame dried round bottomed flask fitted with nitrogen inlet, magnetic stir bar, condenser and a syringe pump apparatus was charged 40 ml of THF, 0.92 ml (6.4 mmol) CBz-Cl and 1.3 gm (7.1 mmol) N-benzylmaleimide. The solution was heated to 45° C. in an oil bath with stirring. A THF solution (7 ml) of 1.43 gm (6.4 mmol) (4-Fluoro-2-methyl-benzylidene)-trimethylsilanylmethyl-amine was added via syringe pump over a period of one hour. The reaction was stirred for two additional hours at 45° C. and then cooled to room temperature. The reaction mixture was concentrated in vacuo. The residue was chromatographed on silica gel eluting with 6/4 ethyl acetate/hexanes to yield two diastereomeric products. Mass spectrum APCI m/z=473 (p+1)

### Example 269

### 5-Benzyl-1-phenyl-4,6-dioxo-hexahydro-pyrrolo[3,4-c]pyrrole-2-carboxylic acid benzyl ester

Using a procedure similar to Example 268; Mass spectrum APCI m/z=441 (p+1)

### Example 270

### 5-Benzyl-1-(2-methyl-phenyl)-4,6-dioxo-hexahydro-pyrrolo[3,4-c]pyrrole-2-carboxylic acid benzyl ester

Using a procedure similar to Example 268; Mass spectrum APCI m/z=455 (p+1)

### Example 271

### 5-Benzyl-1-(4-fluoro-2-methyl-phenyl)-hexahydro-pyrrolo[3,4-c]pyrrole-2-carboxylic acid benzyl ester

To a flame dried flask with nitrogen inlet and stir bar was charged 0.53 gm (1.12 mmol) of the cis isomer of 5-Benzyl-1-(4-fluoro-2-methyl-phenyl)-4,6-dioxo-hexahydro-pyrrolo[3,4-c]pyrrole-2-carboxylic acid benzyl ester prepared above. The starting material was dissolved in anhydrous THF (50 ml) and 90 mg (2.36 mmol) of sodium borohydride was added followed by cooling the reaction mixture to 0° C. Boron trifluoride etherate (0.4 ml 3.14 mmol) was added dropwise over one minute and the reaction was warmed to room temperature and then heated to 80° C. for 4 hours. The reaction mixture was carefully treated with 0.58 gm (6.72 mmol) piperazine portionwise followed by 10 ml of water. The reaction was heated at 80° C. for 16 hours. The oil bath was removed and the reaction mixture was allowed to cool to room temperature. The mixture was diluted with water and extracted with ethyl acetate. The organic phase was dried and evaporated to yield a clear yellow oil (0.486 gm 98%). Mass spectrum APCI m/z=445 (p+1)

### Example 272

### 5-Benzyl-1-(2-methyl-phenyl)-hexahydro-pyrrolo[3,4-c]pyrrole-2-carboxylic acid benzyl ester

Using a procedure similar to Example 271; Mass spectrum APCI m/z=427 (p+1)

### Example 273

### 5-Benzyl-1-phenyl-hexahydro-pyrrolo[3,4-c]pyrrole-2-carboxylic acid benzyl ester

Using a procedure similar to Example 271; Mass spectrum APCI m/z=413 (p+1)

### Example 274

### 5-Benzyl-1-(4-fluoro-2-methyl-phenyl)-octahydro-pyrrolo[3,4-c]pyrrole

5-Benzyl-1-(4-fluoro-2-methyl-phenyl)-hexahydro-pyrrolo[3,4-c]pyrrole-2-carboxylic acid benzyl ester prepared above (0.654 gm 1.47 mmol) and 0.47 ml (5.89 mmol) 48% HBr in acetic acid was stirred for 30 minutes. The dark mixture was treated with 20 ml of diethyl ether whereupon a precipitate formed. The slurry was stirred for 30 minutes and then the mixture was filtered to afford a brown solid. The material was taken up in 2N NaOH and methylene chloride. The organic layer was washed with brine and then dried over sodium sulfate and evaporated in vacuo to afford a clear oil (0.32 gm 70%). Mass spectrum APCI m/z=311 (p+1)

### Example 275

### 5-Benzyl-1-(2-methyl-phenyl)-octahydro-pyrrolo[3,4-c]pyrrole

Using a procedure similar to Example 274; Mass spectrum APCI m/z=293 (p+1)

### Example 276

### 5-Benzyl-1-phenyl-octahydro-pyrrolo[3,4-c]pyrrole

Using a procedure similar to Example 274; Mass spectrum APCI m/z=278 (p+1)

### Example 277

### 5-Benzyl-1-(4-fluoro-2-methyl-phenyl)-hexahydro-pyrrolo[3,4-c]pyrrole-2-carboxylic acid [1-(3,5-bis-trifluoromethyl-phenyl)-ethyl]-methyl-amide

To a flame dried round bottom flask with nitrogen inlet and magnetic stirrer was added 0.213 gm (0.79 mmol.) (R)-[1-(3,5-Bis-trifluoromethyl-phenyl)-ethyl]-methyl-amine (prepared and resolved according to procedure of WO 01/25219) to 50 ml of toluene. The solution was treated with 0.184 ml (1.43 mmol.) triethylamine and 0.085 (0.86 mmol.) 20% phosgene in toluene and was stirred at room temperature for 4 hours. The reaction mixture was then treated with 0.44 gm (0.019) DMAP and 0.184 ml (1.43 mmol.) triethylamine followed by 0.222 gm (0.716) 5-Benzyl-1-(4-fluoro-2-methyl-phenyl)-octahydro-pyrrolo[3,4-c]pyrrole prepared above. The reaction mixture was heated to 100° C. for 16 hours and then allowed to cool to room temperature over 2 hours. The solvent was evaporated in vacuo and the residue was partition between methylene chloride and saturated aqueous sodium bicarbonate solution. The organic layer was washed with water, brine and then dried and evaporated in vacuo. The residue was chromatographed on silica gel eluting with 4 THF/6 petroleum ether to afford 65.6 mg of less polar diastereomer 1 Mass spectrum APCI m/z=607 (p+1) and 47 mg of more polar diastereomer 2. Mass spectrum APCI m/z=607 (p+1)

### Example 278

### 5-Benzyl-1-(2-methylphenyl)-hexahydro-pyrrolo[3,4-c]pyrrole-2-carboxylic acid [1-(3,5-bis-trifluoromethyl-phenyl)-ethyl]-methyl-amide

Using a procedure similar to Example 277; Mass spectrum APCI m/z=590 (p+1)

### Example 279

### 5-Benzyl-1-phenyl-hexahydro-pyrrolo[3,4-c]pyrrole-2-carboxylic acid [1-(3,5-bis-trifluoromethyl-phenyl)-ethyl]-methyl-amide

Using a procedure similar to Example 277; Mass spectrum APCI m/z=576 (p+1)

### Example 280

### 1-(4-Fluoro-2-methyl-phenyl)-hexahydro-pyrrolo[3,4-c]pyrrole-2-carboxylic acid [1-(3,5-bis-trifluoromethyl-phenyl)-ethyl]-methyl-amide

To a flame dried round bottom flask with nitrogen inlet, condenser and stir bar was charged with 0.150 gm (0.25 mmol) 5-Benzyl-1-(4-fluoro-2-methyl-phenyl)-hexahydro-pyrrolo[3,4-c]pyrrole-2-carboxylic acid [1-(3,5-bis-trifluoromethyl-phenyl)-ethyl]-methyl-amide prepared as above. The starting material was dissolved in methanol and treated with 0.078 gm (1.23 mmol) ammonium formate and 0.15 gm 10% palladium on carbon. The mixture was heated to reflux for 30 minutes. The reaction mixture was filtered through celite and the solvent was evaporated in vacuo. The residue was taken up in methylene chloride washed with saturated aqueous sodium bicarbonate solution and then washed with brine, dried over sodium sulfate and evaporated in vacuo to afford a clear oil (115 mg). Mass spectrum APCI m/z=518 (p+1)

### Example 281

### 1-(2-methylphenyl)-hexahydro-pyrrolo[3,4-c]pyrrole-2-carboxylic acid [1-(3,5-bis-trifluoromethyl-phenyl)-ethyl]-methyl-amide

Using a procedure similar to Example 280; Mass spectrum APCI m/z=500 (p+1)

### Example 282

### 1-Phenyl-hexahydro-pyrrolo[3,4-c]pyrrole-2-carboxylic acid [1-(3,5-bis-trifluoromethyl-phenyl)-ethyl]-methyl-amide

Using a procedure similar to Example 280; Mass spectrum APCI m/z=486 (p+1)

### Example 283

### 1-(4-Fluoro-2-methyl-phenyl)-5-(pyrrolidin-1-yl-acetyl)-hexahydro-pyrrolo[3,4-c]pyrrole-2-carboxylic acid [1-(3,5-bis-trifluoromethyl-phenyl)-ethyl]-methyl-amide

To a flame dried round bottomed flask with nitrogen inlet and magnetic stir bar was added 1-(4-Fluoro-2-methyl-phenyl)-hexahydro-pyrrolo[3,4-c]pyrrole-2-carboxylic acid [1-(3,5-bis-trifluoromethyl-phenyl)-ethyl]-methyl-amide (46 mg, 0.089 mmol; prepared above) and Pyrrolidin-1-yl-acetic acid hydrochloride (18 mg; 0.11 mmol) in 5 ml methylene chloride. The mixture was treated with 80 ul (0.445 mmol) Hunig's base and 40 mg (0.089 mmol) of the “BOP reagent CAS [56602-33-6]”. The reaction mixture was stirred at room temperature for 16 hours. The reaction mixture was partitioned between water and ethyl acetate and the organic phase was washed several times with water and then dried over sodium sulfate and evaporated in vacuo. The residue was triturated with ether to afford a yellow solid. Mass spectrum APCI m/z=629 (p+1)

### Example 284

### 2-(2-Methyl-benzyl)-4-oxo-3,4-dihydro-2H-pyridine-1-carboxylic acid benzyl ester

A solution of 2.03 ml (20 mmol) 6-methoxypyridine in 150 ml anhydrous THF was treated dropwise with 10 ml (20 mmol) 2M o-toluyl magnesium bromide in diethyl ether over 10 minutes. The solution was allowed to stir for 1 hour at room temperature and then cooled to −23° C. 2.86 ml (20 mmol) benzyl chloroformate was added drop wise and the reaction mixture was stirred at the same temperature for 1 hour. The mixture was quenched in 200 ml of 10% aqueous HCl and stirred for 16 hours at room temperature. The THF was removed using a rotary evaporator and replaced with an equal volume of ethyl acetate. The organic phase was washed with saturated carbonate solution and then brine. The organic phase was dried over sodium sulfate and the volume was reduced using a rotary evaporator. Hexanes was added to afford a white precipitate. Filtration followed by washing with hexane afforded 5 g (78%) of a pale yellow solid. Mass spectrum APCI m/z=322 (p+1)

### Examples 285

### 2-(4-Fluoro-2-methyl-phenyl)-4-oxo-3,4-dihydro-2H-pyridine-1-carboxylic acid phenyl (and also benzyl) ester

Using a procedure similar to Example 284; Mass spectrum APCI m/z=340 (p+1)

### Example 286

### 2-phenyl-4-oxo-3,4-dihydro-2H-pyridine-1-carboxylic acid phenyl (and also benzyl) ester

Using a procedure similar to Example 284; Mass spectrum APCI m/z=308 (p+1)

### Example 287

### 2-(4-Fluoro-2-methyl-phenyl)-4-oxo-piperidine-1-carboxylic acid benzyl ester

A solution of 3.4 gm (10 mmol) 2-(4-Fluoro-2-methyl-phenyl)-4-oxo-3,4-dihydro-2H-pyridine-1-carboxylic acid benzyl ester in 80 ml of acetic acid was treated with 13 gm (200 mmol) of zinc dust. The reaction mixture was stirred for 20 hours at room temperature. The reaction mixture was filtered and the solid mass was washed with ethyl acetate. The filtrate was evaporated in vacuo and the residue was diluted with water and then basified by carefully adding a saturated aqueous solution of potassium carbonate. The mixture was extracted with ethyl acetate and the organic phase was washed with brine and then dried over sodium sulfate and evaporated in vacuo to afford 3.42 gm (100%) of a yellow oil. Mass spectrum APCI m/z=342 (p+1)

### Example 288

### 2-(2-methyl-phenyl)-4-oxo-piperidine-1-carboxylic acid phenyl (and also benzyl) ester

Using a procedure similar to Example 287; Mass spectrum APCI m/z=324 (p+1)

### Example 289

### 2-(phenyl)-4-oxo-piperidine-1-carboxylic acid phenyl (and also benzyl) ester

Using a procedure similar to Example 287; Mass spectrum APCI m/z=310 (p+1)

### Example 290

### 7-Benzyl-2-(2-methyl-phenyl)-9-oxo-3,7-diaza-bicyclo[3.3.1]nonane-3-carboxylic acid benzyl ester

A solution containing 1.11 ml (9.28 mmol) benzylamine, 1.11 gm (37.12 mmol) paraformaldehyde, 0.55 ml (9.28 mmol) acetic acid, 0.049 ml (0.46 mmol) 35% hydrochloric acid and 50 ml methanol was heated to 65° C. for 1 hour. A second solution consisting of 3 gm (9.28 mmol) 2-(2-methyl-phenyl)-4-oxo-piperidine-1-carboxylic acid benzyl ester and 0.55 ml (9.28 mmol) acetic acid in 50 ml of methanol was then added drop wise and the resultant mixture was heated under reflux for 16 hours. The reaction mixture was cooled to room temperature and the solvent was removed in vacuo. The residue was diluted with water and then basified by carefully adding a saturated aqueous solution of potassium carbonate. The mixture was extracted with ethyl acetate and the organic phase was washed with water, brine and then dried over sodium sulfate and evaporated in vacuo. The residue was chromatographed on silica gel eluting with 10% -35% ethyl acetate in hexane to afford 2.25 gm (53%) of a white foam. Mass spectrum APCI m/z=455 (p+1)

### Example 291

### 7-Benzyl-2-(4-fluoro-2-methyl-phenyl)-9-oxo-3,7-diaza-bicyclo[3.3.1]nonane-3-carboxylic acid benzyl

Using a procedure similar to Example 290; Mass spectrum APCI m/z=473 (p+1) (and also phenyl) ester Mass spectrum APCI m/z=459 (p+1)

### Example 292

### 7-Benzyl-2-phenyl-9-oxo-3,7-diaza-bicyclo[3.3.1]nonane-3-carboxylic acid benzyl

Using a procedure similar to Example 290; Mass spectrum APCI m/z=441 (p+1) (and also phenyl) ester Mass spectrum APCI m/z=427 (p+1)

### Example 293

### 7-Benzyl-2-(4-fluoro-2-methylphenyl)-3,7-diazabicyclo[3.3.1]nonane-3-carboxylic acid phenyl ester

In a single neck round bottom flask fitted with a condenser and nitrogen inlet was combined 0.205 gm (1.1 mmol) tosylhydrazine and 0.458 gm (1 mmol) 7-Benzyl-2-(4-fluoro-2-methylphenyl)-9-oxo-3,7-diaza-bicyclo[3.3.1]nonane-3-carboxylic acid phenyl ester in 20 ml methanol. The reaction mixture was heated to 65° C. for 2 hours and then allowed to cool to room temperature and was stirred for 16 hours. The solution was evaporated in vacuo to afford a white solid that was used without purification.

To a flame dried round bottomed flask was charged the tosylhydrazone [62 mg (0.1 mmol)] prepared above and 2 ml chloroform. This was followed by addition of 16 ul (0.15 mmol) catecholborane and the reaction mixture was stirred for 2 hours at room temperature. The mixture was treated with 82 mg (0.6 mmol) sodium acetate whereupon gas evolution was observed. The mixture was stirred for 16 hours at room temperature. The solvent was removed in vacuo and the residue was treated with 5 ml of methanol and the mixture was refluxed for 1 hour. The reaction mixture was cooled to room temperature and evaporated in vacuo. The residue was taken up in methylene chloride, filtered through celite, and the filtrate was evaporated in vacuo. The residue was chromatographed on silica gel (elution with 20% ethyl acetate in hexane) to afford 17 mg (39%) of the desired product. Mass spectrum APCI m/z=445 (p+1)

### Example 294

### 7-Benzyl-2-(2-methylphenyl)-3,7-diazabicyclo[3.3.1]nonane-3-carboxylic acid phenyl

Using a procedure similar to Example 293; Mass spectrum APCI m/z=427 (p+1) (and benzyl) ester Mass spectrum APCI m/z=441 (p+1)

### Example 295

### 7-Benzyl-2-phenyl-3,7-diazabicyclo[3.3.1]nonane-3-carboxylic acid phenyl

Using a procedure similar to Example 293; Mass spectrum APCI m/z=413 (p+1) (and benzyl) ester; Mass spectrum APCI m/z=427 (p+1)

### Example 296

### 7-Benzyl-2-(4-fluoro-2-methyl-phenyl)-3,7-diaza-bicyclo[3.3.1 ]nonane

from phenylcarbamate: To a solution of 0.4 gm (7.2 mmol) potassium hydroxide in 10 ml of ethanol was added 80 mg (0.18 mmol) 7-Benzyl-2-(4-fluoro-2-methylphenyl)-3,7-diazabicyclo[3.3.1]nonane-3-carboxylic acid phenyl ester. The reaction was heated to reflux for 16 hours. The reaction mixture was allowed to cool to room temperature and was then concentrated in vacuo. The residue was diluted with water and extracted with methylene chloride. The organic phase was washed with brine and dried over sodium sulfate and evaporated. Chromatography on silica gel (elution with 15% methanol in methylene chloride with 1% added ammonium hydroxide) afforded 58 mg (100%) of a colorless oil.

from benzyl carbamate: To 250 mg (0.56 mmol) 7-Benzyl-2-(2-methylphenyl)-3,7-diazabicyclo[3.3.1]nonane-3-carboxylic acid benzyl ester at 0° C. was added 0.5 ml of a solution of 30% HBr in acetic acid. The mixture was stirred for 2 hours and was then diluted with ether. The mixture was concentrated in vacuo and the residue was taken up in water. The mixture was made basic to pH 10 with 2N sodium hydroxide and was then extracted with methylene chloride. The organic phase was washed with water, brine and then dried over sodium sulfate and evaporated in vacuo. Chromatography on silica gel (elution with 3% methanol in methylene chloride with 1% added ammonium hydroxide) afforded 41 mg (24%) of a pale yellow oil.

Mass spectrum APCI m/z=325 (p+1)

### Example 297

### 7-Benzyl-2-(2-methyl-phenyl)-3,7-diaza-bicyclo[3.3.1]nonane

Using a procedure similar to Example 296; Mass spectrum APCI m/z=307 (p+1)

### Example 298

### 7-Benzyl-2-phenyl-3,7-diaza-bicyclo[3.3.1]nonane

Using a procedure similar to Example 296; Mass spectrum APCI m/z=293 (p+1)

### Example 299

### 7-Benzyl-2-(4-fluoro-2-methyl-phenyl)-3,7-diaza-bicyclo[3.3.1]nonane-3-carboxylic acid [1-(3,5-bis-trifluoromethyl-phenyl)-ethyl]-methyl-amide

To a flame dried round bottom flask with nitrogen inlet and magnetic stirrer was added 0.236 gm (0.87 mmol.) (R)-[1-(3,5-Bis-trifluoromethyl-phenyl)-ethyl]-methyl-amine (prepared and resolved according to procedure of WO 01/25219) to 5 ml of toluene. The solution was treated with 50 ul (0.36 mmol.) triethylamine and 455 ul (0.92 mmol.) 20% phosgene in toluene and was stirred at room temperature for 4 hours. The reaction mixture was then treated with 0.18 gm (0.026 mmol) PS-DMAP and 50 ul (0.36 mmol.) triethylamine followed by 0.130 gm (0.4 mmol) 7-Benzyl-2-(4-fluoro-2-methyl-phenyl)-3,7-diaza-bicyclo [3.3.1]nonane prepared above. The reaction mixture was heated to 100° C. for 16 hours and then treated with 100 ul (0.72 mmol) triethyl amine and heated an additional 4 hours and then finally allowed to cool to room temperature over 2 hours. The solvent was evaporated in vacuo and the residue was partition between methylene chloride and saturated aqueous sodium bicarbonate solution. The organic layer was washed with water, brine and then dried and evaporated in vacuo. The residue was chromatographed on silica gel eluting with 10-30% ethyl acetate in hexanes to afford 205 mg (82%) of a pale yellow solid. Mass spectrum APCI m/z=622 (p+1)

### Example 300

### 7-Benzyl-2-(2-methyl-phenyl)-3,7-diaza-bicyclo[3.3.1]nonane-3-carboxylic acid [1-(3,5-bis-trifluoromethyl-phenyl)-ethyl]-methyl-amide

Using a procedure similar to Example 299; Mass spectrum APCI m/z=604 (p+1)

### Example 301

### 7-Benzyl-2-phenyl-3,7-diaza-bicyclo[3.3.1]nonane-3-carboxylic acid [1-(3,5-bis-trifluoromethyl-phenyl)-ethyl]-methyl-amide

Using a procedure similar to Example 299; Mass spectrum APCI m/z=590 (p+1)

### Example 302

### 2-(4-Fluoro-2-methyl-phenyl)-3,7-diaza-bicyclo[3.3.1]nonane-3-carboxylic acid [1-(3,5-bis-trifluoromethyl-phenyl)-ethyl]-methyl-amide

To a flame dried round bottom flask with nitrogen inlet, condenser and stir bar was charged with 0.205 gm (0.33 mmol) 7-Benzyl-2-(4-fluoro-2-methyl-phenyl)-3,7-diaza-bicyclo[3.3.1]nonane-3-carboxylic acid [1-(3,5-bis-trifluoromethyl-phenyl)-ethyl]-methyl-amide prepared as above. The starting material was dissolved in 20 ml methanol and treated with 0.83 gm (13.2 mmol) ammonium formate and 0.140 gm 10% palladium on carbon. The mixture was heated to reflux for 120 minutes. The reaction mixture was filtered through celite and the solvent was evaporated in vacuo. The residue was taken up in methylene chloride washed with saturated aqueous sodium bicarbonate solution and then washed with brine, dried over sodium sulfate and evaporated in vacuo. The residue was chromatographed on silica gel eluting with 2-10% methanol in methylene chloride with 1% ammonium hydroxide to afford 177 mg (100%) of a pale yellow solid. Mass spectrum APCI m/z=532 (p+1)

### Example 303

### 2-(2-methyl-phenyl)-3,7-diaza-bicyclo[3.3.1]nonane-3-carboxylic acid [1-(3,5-bis-trifluoro methyl-phenyl)-ethyl]-methyl-amide

Using a procedure similar to Example 302; Mass spectrum APCI m/z=514 (p+1)

### Example 304

### 2-phenyl-3,7-diaza-bicyclo[3.3.1]nonane-3-carboxylic acid [1-(3,5-bis-trifluoro methyl-phenyl)-ethyl]-methyl-amide

Using a procedure similar to Example 302; Mass spectrum APCI m/z=500 (p+1)

### Example 305

### 1-{4-[2-Benzyl-1-(3,5-bis-trifluoromethyl-benzoyl)-piperidin-3-ylamino]-piperidin-1-yl}-ethanone

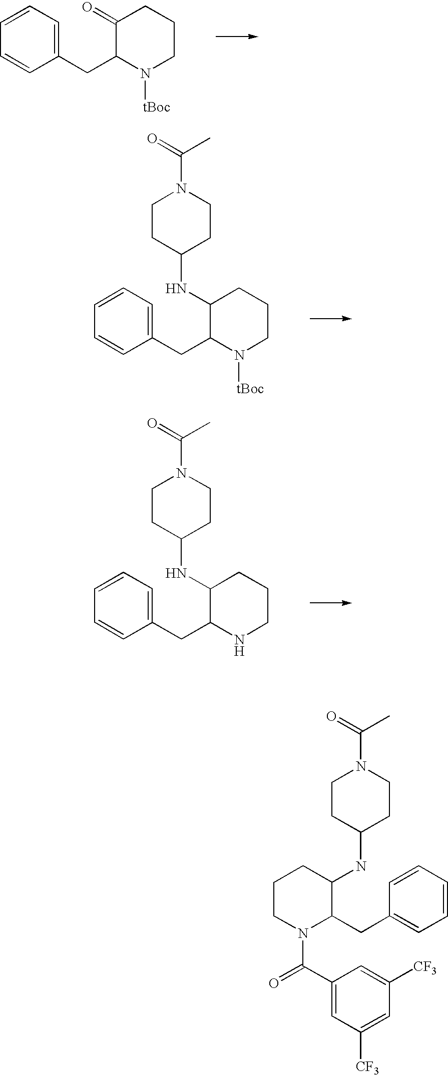

### Example 306

### 2-Benzyl-3-oxopiperidine-1-carboxylic acid tert-butyl ester

Prepared according to the procedure of Brubaker and Colley: J. Medicinal Chem. 1986, 29, 1528-1531 from commercially available 3-Oxo-piperidine-1-carboxylic acid tert-butyl ester.

### Example 307

### 1-(4-Amino-piperidin-1-yl)-ethanone

1-Acetyl-piperidin-4-one (10 gm (70 mmol)) was dissolved in 200 ml ethanol and treated with 10 gm (143 mmol) hydroxylamine hydrochloride and 10 ml pyridine. The reaction mixture was heated to 70° C. for 1.5 hr. The solvent was removed and the residue was treated with water and cooled to 0° C. The resulting slurry was filtered and dried under vacuum to afford 6.5 gm 1-Acetyl-piperidin-4-one oxime as a white solid (59%). The oxime (2.0 gm (12 mmol)) in a Parr bottle was dissolved in 50 ml of ethanol and treated with 0.2 gm of Raney nickel which had been washed several times with water. The reaction was placed in to Parr apparatus and hydrogenated under 50 psi hydrogen pressure for 16 hours. The mixture was carefully filtered and the filtrate was evaporated in vacuo to afford 1.7 gm (100%) of the desired amine as a green solid. GC Mass spectrum m/z=142

### Example 308

### 3-(1-Acetyl-piperidin-4-ylamino)-2-benzyl-piperidine-1-carboxylic acid tert-butyl ester

A solution of 0.53 gm (3.72 mmol) 1-(4-Amino-piperidin-1-yl)-ethanone and 1.07 gm (3.72 mmol) 2-Benzyl-3-oxopiperidine-1-carboxylic acid tert-butyl ester in toluene was heated under reflux over a Dean-Stark water separator for 18 hours. The reaction mixture was cooled to room temperature and was treated with 2.37 gm (11.2 mmol) sodium triacetoxyborohydride. The reaction mixture was then stirred for 20 hours at ambient temperature. The reaction mixture was quenched by adding saturated bicarbonate solution and then extracted with methylene chloride. The organic phase was washed with water and saturated brine. The organic phase was then dried over sodium sulfate and evaporated in vacuo to afford a yellow oil. The oil was chromatographed on silica gel (elution with 5% methanol in methylene chloride with 1 ml/100 ml of conc ammonium hydroxide) to afford 890 mg (58%) of an oil. Mass spectrum APCI m/z=416 (p+1)

### Example 309

### 1-[4-(2-Benzyl-piperidin-3-ylamino)-piperidin-1-yl]-ethanone

A solution of 890 mg (2.14 mmol) 3-(1-Acetyl-piperidin-4-ylamino)-2-benzyl-piperidine-1-carboxylic acid tert-butyl ester in 25 ml dichloroethane was treated with 1.65 ml (21.4 mmol) trifluoroacetic acid then heated under reflux for 2 hr. The reaction mixture was cooled to ambient temperature and evaporated in vacuo. The residue was diluted with water and basified by adding 2 N sodium hydroxide solution to pH 9.0 and then extracted with methylene chloride. The organic phase was washed with water and saturated brine. The organic phase was then dried over sodium sulfate and evaporated in vacuo to afford 640 mg (95%) of an oil. Mass spectrum APCI m/z=316 (p+1)

### Example 310

### 1-{4-[2-Benzyl-1-(3,5-bis-trifluoromethyl-benzoyl)-piperidin-3-ylamino]-piperidin-1-yl}-ethanone

A solution of 640 mg (2.03 mmol) 1-[4-(2-Benzyl-piperidin-3-ylamino)-piperidin-1-yl]-ethanone in 56 ml dichloroethane was treated with 1.13 ml (8.12 mmol) triethylamine, 68 mg (0.12 mmol) PS-DMAP followed by 0.37 ml (2.03 mmol) 3,5-Bis-trifluoromethyl-benzoyl chloride at room temperature for 20 hours. The reaction mixture was diluted with methylene chloride. The organic phase was washed with water and saturated brine. The organic phase was then dried over sodium sulfate and evaporated in vacuo. The residue was chromatographed on silica gel (elution with 5% methanol in methylene chloride with 1 ml/100 ml of conc ammonium hydroxide) to afford 1.0 gm (89%) of a pale yellow foam. Mass spectrum APCI m/z=556 (p+1) This compound is a mixture of cis and trans isomers which was separated by chiral chromatography into a mixture of cis and trans enantiomeric pairs. Chiralcel OD (4.6 mm×25 cm) 80/20 Heptane/EtOH at 1 ml/min. Retention times: 6.0 min, 6.8 min, 7.5 min and 9.3 min.

### Example 311

### 1-{4-[2-Benzyl-1-(3,5-bis-trifluoromethyl-benzoyl)-piperidin-3-ylmethyl]-piperazin-1-yl}-ethanone

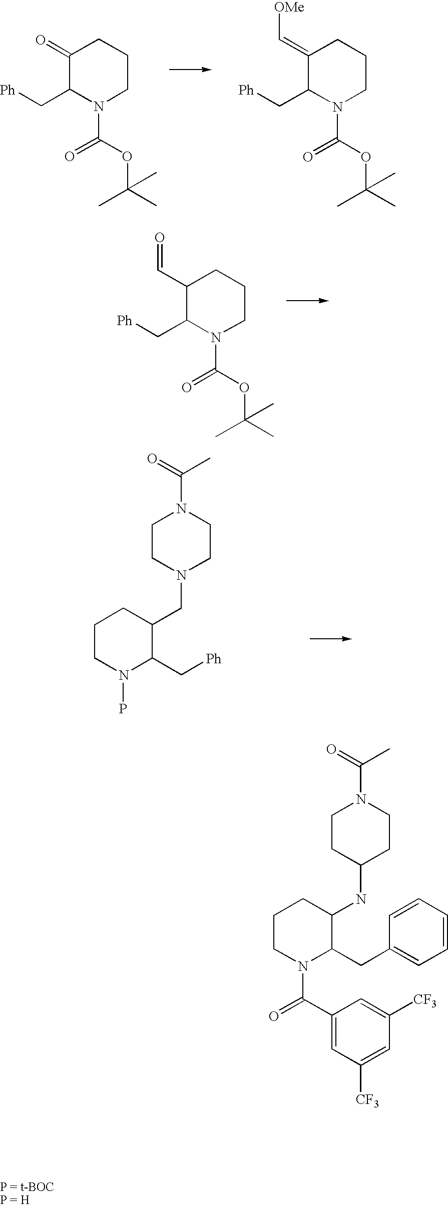

### Example 312

### 2-Benzyl-3-oxopiperidine-1-carboxylic acid tert-butyl ester

Prepared according to the procedure of Brubaker and Colley: J. Medicinal Chem. 1986, 29, 1528-1531 from commercially available 3-Oxo-piperidine-1-carboxylic acid tert-butyl ester.

### Example 313

### 2-Benzyl-3-methoxymethylene-piperidine-1-carboxylic acid tert-butyl ester

To a stirred solution of 1.82 gm (5.3 mmol) methoxymethytriphenylphosphonium chloride in 48 ml anhydrous THF at −78° C. was added a solution of lithium diisopropylamide (prepared at −78° C. with 1.6 ml (4 mmol) n butyl lithium and 0.56 ml (4 mmol) diisopropyl amine) in 4 ml of anhydrous THF. The reaction mixture was stirred for 40 min at −78° C. and then a solution of 722 mg (2.5 mmol) 2-Benzyl-3-oxopiperidine-1-carboxylic acid tert-butyl ester in 12 ml of anhydrous THF was added. The mixture was stirred for 10 min at −78° C. and then allowed to warm to room temperature and stirred for 1.5 hours. The reaction mixture was then heated under reflux for 16 hours. The reaction mixture was cooled and quenched by adding saturated brine solution and then extracted with methylene chloride. The organic phase was washed with saturated sodium bicarbonate solution followed by water and saturated brine. The organic phase was then dried over sodium sulfate and evaporated in vacuo to afford a yellow oil. The oil was chromatographed on silica gel (elution with 20% ethyl acetate in hexane) to afford 512 mg (64%) of an oil. Mass spectrum APCI m/z=318 (p+1)

### Example 314

### 2-Benzyl-3-formyl-piperidine-1-carboxylic acid tert-butyl ester

A room temperature solution of 15 ml 3M aqueous HCl and 20 ml of THF was treated with 512 mg (1.64 mmol) of 2-Benzyl-3-methoxymethylene-piperidine-1-carboxylic acid tert-butyl ester and stirred for 6 hours. The reaction mixture was extracted with methylene chloride. The organic phase was washed with saturated sodium bicarbonate solution followed by saturated brine and then dried over sodium sulfate and evaporated in vacuo to afford 495 mg of a yellow oil (100%). Mass spectrum APCI m/z=303 (p+1)

### Example 315

### 3-(4-Acetyl-piperazin-1-ylmethyl)-2-benzyl-piperidine-1-carboxylic acid tert-butyl ester

A solution of 495 mg (1.64 mmol) 2-Benzyl-3-formyl-piperidine-1-carboxylic acid tert-butyl ester and 630 mg (4.93 mmol) 1-Piperazin-1-yl-ethanone in 200 ml dichloroethane was stirred at ambient temperature under nitrogen for 15 minutes before adding 2.1 gm (9.84 mmol) sodium triacetoxyborohydride portion wise over 2 minutes. The reaction mixture was then stirred for 20 hours at ambient temperature. The reaction mixture was quenched by adding saturated bicarbonate solution and then extracted with methylene chloride. The organic phase was washed with water and saturated brine. The organic phase was then dried over sodium sulfate and evaporated in vacuo to afford a yellow oil. The oil was chromatographed on silica gel (elution with 2% methanol in methylene chloride with 1 ml/100 ml of conc ammonium hydroxide) to afford 533 mg (78%) of an oil. Mass spectrum APCI m/z=416 (p+1)

### Example 316

### 1-[4-(2-Benzyl-piperidin-3-ylmethyl)-piperazin-1-yl]-ethanone

A solution of 533 mg (1.28 mmol) 3-(4-Acetyl-piperazin-1-ylmethyl)-2-benzyl-piperidine-1-carboxylic acid tert-butyl ester in 25 ml dichloroethane was treated with 1.0 ml (12.8 mmol) trifluoroacetic acid then heated under reflux for 1 hr. The reaction mixture was cooled to ambient temperature. The reaction mixture was basified by adding 1N sodium hydroxide solution to pH 9.0 and then extracted with methylene chloride. The organic phase was washed with water and saturated brine. The organic phase was then dried over sodium sulfate and evaporated in vacuo to afford 392 mg (97%) of an oil. Mass spectrum APCI m/z=316 (p+1)

### Example 317

### 1-{4-[2-Benzyl-1-(3,5-bis-trifluoromethyl-benzoyl)-piperidin-3-ylmethyl]-piperazin-1-yl}-ethanone

A solution of 392 mg (1.24 mmol) 1-[4-(2-Benzyl-piperidin-3-ylmethyl)-piperazin-1-yl]-ethanone in 25 ml dichloroethane was treated with 0.52 ml (3.72 mmol) triethylamine, 68 mg (0.12 mmol) PS-DMAP followed by 0.23 ml (1.24 mmol) 3,5-Bis-trifluoromethyl-benzoyl chloride at room temperature for 20 hours. The reaction mixture was diluted with methylene chloride. The organic phase was washed with water and saturated brine. The organic phase was then dried over sodium sulfate and evaporated in vacuo. The residue was chromatographed on silica gel (gradient elution with 80% ethyl acetate in hexane followed by 5% methanol in methylene chloride followed by 10% methanol in methylene chloride with 1 ml/100 ml of conc ammonium hydroxide) to afford 432 mg (63%) of a pale yellow foam. Mass spectrum APCI m/z=556 (p+1). This compound is a mixture of cis and trans isomers which was separated by chiral chromatography into a mixture of cis and trans enantiomeric pairs. Chiralcel OD (4.6 mm×25 cm) 85/15 Heptane/EtOH 0.1% TFA at 1 ml/min. Retention times: 8.9 min, 11.6 min, 14.5 min and 17.9 min.

1. A compound having the formula: 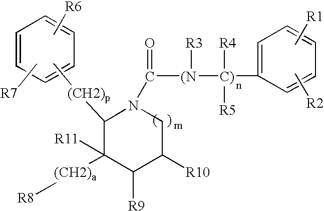
or pharmaceutically acceptable salts and solvates thereof, the (R) and (S) enantiomers thereof and the cis and trans isomers thereof
wherein
m=0 or 1; n=0 or 1; p=0, 1, 2 or 3; a=0, 1, 2 or 3;
R1 and R2 are each independently C₁₋₆alkyl, C₁₋₆alkoxy, —CF₃, —OCF₃, or halogen;
R3 is hydrogen or C₁₋₆alkyl;
R4 is hydrogen, C₁₋₆alkyl, C₂₋₆alkenyl, C₃₋₇cycloalkyl, or R4 and R3 together with the C and N atoms to which they are respectively attached form a 5 to 6 member heterocyclic group;
R5 is hydrogen, C₁₋₆alkyl, or R5 and R4 together with the C atom to which they are attached form a C₃₋₇cycloalkyl;
R6 and R7 are each independently hydrogen, halogen or C₁₋₆alkyl;
R9 and R10 are each independently hydrogen, C₁₋₆alkyl or, when m=1, R10 and R8 together with R9 and the C atoms to which they are respectively attached may form a 8 to 14 member heterobicyclic ring;
R11 is hydrogen or R11 and R9 together with the C atoms to which they are respectively attached form a C₃₋₇cycloalkyl or, when m=0 and R10 is hydrogen, R9 and R11 together with the C atoms to which they are respectively attached form a 5 to 7 member heterocyclic ring;
R8 is:
i) hydrogen, a C₁₋₆alkyl group, or a C₁₋₇acyl group, either of which groups may be optionally substituted with one or more hydroxy, amino, C₁₋₆alkoxy or substituted with a 4 to 8 member heterocyclic ring or a 5 to 7 member heteroaryl ring either of which rings may be optionally substituted with one or more C₁₋₄alkyl, amino, hydroxy, C₁₋₆alkoxy or C₁₋₇acyl; 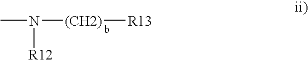
wherein b=0, 1, 2 or 3 and R12 and R13 are each independently hydrogen or one of the following groups: C₁₋₆alkyl, C₃₋₇cycloalkyl, C₁₋₆alkoxycarbonyl, C₅₋₁₀aryl, C₁₋₆alkoxy, C₁₋₇acyl, amino, amido, C₁₋₇acylamino, a 4 to 8 member heterocyclic ring, a 5 to 7 member heteroaryl ring or a C₆₋₁₄heterobicyclic ring, any one of which groups may be optionally substituted with one or more hydroxy, halogen, oxo, C₁₋₇acyl, amino, morpholino or C₁₋₄alkyl; 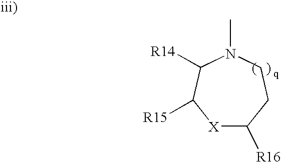
wherein q=0 or 1
R14, R15 and R16 are each independently hydrogen, C₁₋₄alkyl or oxo;
X is O, S or NR17, wherein R17 is hydrogen, C₁₋₆alkyl, C₃₋₇cycloalkyl, C₅₋₁₀aryl, C₁₋₆alkoxycarbonyl, C₁₋₇acyl, amido, or 5 to 7 member heteroaryl ring, any one of which may be optionally substituted with one or more hydroxy, halogen, C₁₋₄alkyl or C₁₋₄alkoxy; or R17 and R15 together with the N and C atoms to which they are attached respectively form a 5 to 8 member heterocyclic ring or a 5 to 7 member heteroaryl ring, either of which rings may be optionally substituted with one or more hydroxy or C₁₋₄alkyl; 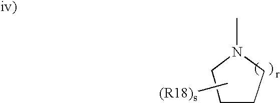
wherein
r=0, 1, 2, 3 or 4
s=0, 1, 2 or 3
each R¹⁸ is individually hyrogen, hydroxyl, C₅₋₁₀aryl, C₁₋₇acyl, amino, piperidinyl, oxadiazolyl, C₁₋₆alkoxy which alkoxy may be optionally substituted with an amido or C₁₋₆alkyl which alkyl may be optionally substituted with an alkoxy, amino, hydroxy or pyrrolyl group; or when m=0 R⁸ and R⁹ together with the C atoms to which they are attached may form a 5-member heterocyclic ring which heterocyclic ring may be optionally substituted with
a) C₁₋₆alkyl which alkyl may be optionally substituted with C₅₋₁₀aryl, or
b) a group of the formula 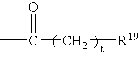
wherein t=0, 1 or 2 and R¹⁹ is a 4 to 8 member heterocyclic ring.

2. The compound of claim 1 wherein m=0, n=1, p=0, a=0 or 1; R1 and R2 are each —CF₃; R3 and R4 are each C₁₋₆alkyl; R5, R9 and R10 are each hydrogen; R6 is C₁₋₆alkyl, R7 is halogen.

3. The compound of claim 2 wherein R3, R4 and R6 are each methyl; R7 is F; and R8 is (i).

4. The compound of claim 2 wherein a=0; R3, R4 and R6 are each methyl; R7 is F; and R8 is (ii).

5. The compound of claim 2 wherein a=1, R3, R4 and R6 are each methyl; R7 is F; and R8 is (ii).

6. The compound of claim 2 wherein a=1, R3, R4 and R6 are each methyl; R7 is F; and R8 is (iii).

7. The compound of claim 2 wherein a=0, R3 and R4 are each C₁₋₃alkyl; R6 Is methyl; R9 and R11 together with the C atoms to which they are respectively attached form a 5 to 7 member heterocyclic ring; R7 is F; and R8 is (i).

8. The compound of claim 7 wherein R8 is hydrogen.

9. The compound of claim 1 wherein m=1, n=1, p=0, a=0 or 1; R1 and R2 are each —CF₃; R3 and R4 are each C₁₋₆alkyl; R5, R9 and R10 are each hydrogen; R6 is C₁₋₆alkyl; R7 is halogen; and R8 is (i).

10. The compound of claim 1 wherein m=1, n=1, p=0, a=1; R1 and R2 are each CF₃; R3 and R4 are each C₁₋₃alkyl; R5, R9 and R11 are each hydrogen; and R8 and R10 together with R9 and the C atoms to which they are respectively attached form an 8 to 14 member heterobicyclic ring.

11. The compund of claim 1 wherein m=1, n=0, p=1, a=0; R1 and R2 are each CF₃; R9, R10 and R11 are each hydrogen; and R8 is (ii).

12. The compound of claim 1 wherein m=1, n=0, p=1, a=1; R1 and R2 are each CF₃; R9, R10 and R11 are each hydrogen; and R8 is (iii).

13. The compound of claim 1 comprising:
2-(4-Fluoro-2-methyl-phenyl)-3-hydroxymethyl-pyrrolidine-1-carboxylic acid [1-(3,5-bis-trifluoromethyl-phenyl)-ethyl]-methyl-amide
2-(4-Fluoro-2-methyl-phenyl)-3-hydroxymethyl-pyrrolidine-1-carboxylic acid [1-(R)-(3,5-bis-trifluoromethyl-phenyl)-ethyl]-methyl-amide
2-(S)-(4-Fluoro-2-methyl-phenyl)-3-(S)-hydroxymethyl-pyrrolidine-carboxylic acid [1-(R)-(3,5-bis-trifluoromethyl-phenyl)-ethyl]-methyl-amide
2-(R)-(4-Fluoro-2-methyl-phenyl)-3-(R)-hydroxymethyl-pyrrolidine-1-carboxylic acid [1-(R)-(3,5-bis-trifluoromethyl-phenyl)-ethyl]-methyl-amide
2-(S)-(4-Fluoro-2-methyl-phenyl)-3-(S)-hydroxymethyl-pyrrolidine-1-(S)-carboxylic acid [1-(3,5-bis-trifluoromethyl-phenyl)-ethyl]-methyl-amide
2-(R)-(4-Fluoro-2-methyl-phenyl)-3-(R)-hydroxymethyl-pyrrolidine-1-(S)-carboxylic acid [1-(3,5-bis-trifluoromethyl-phenyl)-ethyl]-methyl-amide
2-(4-Fluoro-2-methyl-phenyl)-3-formyl-pyrrolidine-1-carboxylic acid [1-(R)-(3,5-bis-trifluoromethyl-phenyl)-ethyl]-methyl-amide
2-(R)-(4-Fluoro-2-methyl-phenyl)-3-(R)-formyl-pyrrolidine-1-carboxylic acid [1-(R)-(3,5-bis-trifluoromethyl-phenyl)-ethyl]-methyl-amide
2-(R)-(4-Fluoro-2-methyl-phenyl)-3-(R)-formyl-pyrrolidine-1-carboxylic acid [1-(S)-(3,5-bis-trifluoromethyl-phenyl)-ethyl]-methyl-amide
2-(S)-(4-Fluoro-2-methyl-phenyl)-3-(S)-formyl-pyrrolidine-1-carboxylic acid [1-(S)-(3,5-bis-trifluoromethyl-phenyl)-ethyl]-methyl-amide
2-(S)-(4-Fluoro-2-methyl-phenyl)-3-(S)-formyl-pyrrolidine-1-carboxylic acid [1-(R)-(3,5-bis-trifluoromethyl-phenyl)-ethyl]-methyl-amide
[1-{[1-(3,5-Bis-trifluoromethyl-phenyl)-ethyl]-methyl-carbamoyl}-2-(4-fluoro-2-methyl-phenyl)-piperidin-3-yl]-carbamic acid tert-butyl ester
2-(R)-(4-Fluoro-2-methyl-phenyl)-3-(R)-hydroxymethyl-piperidine-1-carboxylic acid [1-(R)-(3,5-bis-trifluoromethyl-phenyl)-ethyl]-methyl-amide
2-(R)-(4-Fluoro-2-methyl-phenyl)-3-(R)-formyl-piperidine-1-carboxylic acid [1-(R)-(3,5-bis-trifluoromethyl-phenyl)-ethyl]-methyl-amide
(1-{[1-(3,5-Bis-trifluoromethyl-phenyl)-ethyl]-methyl-carbamoyl}-2-(S)-phenyl-piperidin-3-(S)-yl)-carbamic acid tert-butyl ester
[1-(3,5-Bis-trifluoromethyl-benzylcarbamoyl)-2-(S)-phenyl-piperidin-3-(S)-yl]-carbamic acid tert-butyl ester
(1-{[1-(R)-(3,5-Bis-trifluoromethyl-phenyl)-ethyl]-methyl-carbamoyl}-2-(R)-phenyl-piperidin-3-yl)-carbamic acid tert-butyl ester
Trans-(1-{[1-(3,5-Bis-trifluoromethyl-phenyl)-ethyl]-methyl-carbamoyl}-2-phenyl-piperidin-3-yl)-carbamic acid tert-butyl ester
Trans-(1-{[1-(3,5-Bis-trifluoromethyl-phenyl)-ethyl]-methyl-carbamoyl}-2-phenyl-piperidin-3-yl)-methyl-carbamic acid tert-butyl ester
(1-{[1-(R)-(3,5-Bis-trifluoromethyl-phenyl)-ethyl]-methyl-carbamoyl}-2-o-tolyl-piperidin-3-yl)-carbamic acid tert-butyl ester
3-Amino-2-o-tolyl-piperidine-1-carboxylic acid [1-(3,5-bis-trifluoromethyl-phenyl)-ethyl]-methyl-amide
(1-{[1-(3,5-Bis-trifluoromethyl-phenyl)-ethyl]-methyl-carbamoyl}-2-o-tolyl-piperidin-3-yl)-methyl-carbamic acid tert-butyl ester
3-Methylamino-2-o-tolyl-piperidine-1-carboxylic acid [1-(3,5-bis-trifluoromethyl-phenyl)-ethyl]-methyl-amide
(1-(R)-{[1-(3,5-Bis-trifluoromethyl-phenyl)-ethyl]-methyl-carbamoyl}-2-(S)-o-tolyl-pyrrolidin-3-(R)-yl)-carbamic acid tert-butyl ester
(1-{[1-(R)-(3,5-Bis-trifluoromethyl-phenyl)-ethyl]-methyl-carbamoyl}-2-(S)-o-tolyl-pyrrolidin-3-(R)-yl)-methyl-carbamic acid tert-butyl ester
3-(R)-Amino-2-(S)-o-tolyl-pyrrolidine-1-(R)-carboxylic acid [1-(3,5-bis-trifluoromethyl-phenyl)-ethyl]-methyl-amide
2-(4-Fluoro-2-methyl-phenyl)-3-hydroxymethyl-pyrrolidine-1-carboxylic acid [1-(3,5-bis-trifluoromethyl-phenyl)-ethyl]-methyl-amide
2-(4-Fluoro-2-methyl-phenyl)-3-hydroxymethyl-pyrrolidine-1-carboxylic acid [1-(R)-(3,5-bis-trifluoromethyl-phenyl)-ethyl]-methyl-amide
2-(S)-(4-Fluoro-2-methyl-phenyl)-3-(S)-hydroxymethyl-pyrrolidine-1-carboxylic acid [1-(R)-(3,5-bis-trifluoromethyl-phenyl)-ethyl]-methyl-amide
2-(R)-(4-Fluoro-2-methyl-phenyl)-3-(R)-hydroxymethyl-pyrrolidine-1-carboxylic acid [1-(R)-(3,5-bis-trifluoromethyl-phenyl)-ethyl]-methyl-amide
2-(S)-(4-Fluoro-2-methyl-phenyl)-3-(S)-hydroxymethyl-pyrrolidine-1-(S)-carboxylic acid [1-(3,5-bis-trifluoromethyl-phenyl)-ethyl]-methyl-amide
2-(R)-(4-Fluoro-2-methyl-phenyl)-3-(R)-hydroxymethyl-pyrrolidine-1-(S)-carboxylic acid [1-(3,5-bis-trifluoromethyl-phenyl)-ethyl]-methyl-amide
3-(S)-Aminomethyl-2-(R)-(4-fluoro-2-methyl-phenyl)-pyrrolidine-1-carboxylic acid [1-(R)-(3,5-bis-trifluoromethyl-phenyl)-ethyl]-methyl amide
3-(R)-Dimethylaminomethyl-2-(S)-(4-fluoro-2-methyl-phenyl)-pyrrolidine-1-carboxylic acid [1-(R)-(3,5-bis-trifluoromethyl-phenyl)-ethyl]-methyl-amide
2-(R)-(4-Fluoro-2-methyl-phenyl)-3-(S)-methylaminomethyl-pyrrolidine-1-carboxylic acid [1-(S)-(3,5-bistrifluoromethyl-phenyl)-ethyl]-methyl-amide
3-(S)-Dimethylaminomethyl-2-(R)-(4-fluoro-2-methyl-phenyl)-pyrrolidine-1-carboxylic acid [1-(S)-(3,50bix-trifluoromethyl-phenyl)-ethyl]-methyl-amide
2-(S)-(4-Fluoro-2-methyl-phenyl)-3-(R)-[(isopropyl-methyl-amino)-methyl]-pyrrolidine-1-carboxylic acid [1-(S)-(3,5-bis-trifluoromethyl-phenyl)-ethyl]-methyl-amide
3-(R)-(4-Ethyl-piperazin-1-ylmethyl)-2-(S)-(4-fluoro-2-methyl-phenyl)-pyrrolidine-1-carboxylic acid [1-(S)-(3,5-bis-trifluoromethyl-phenyl)-ethyl]-methyl-amide
3-(R)-Azetidin-1-ylmethyl-2-(S)-(4-fluoro-2-metyl-phenyl)-pyrrolidine-1-carboxylic acid [1-(S)-(3,5-bistrifluoromethyl-phenyl)-ethyl]-methyl-amide
3-(R)-Cyclopropylaminomethyl-2-(S)-(4-fluoro-2-methyl-phenyl)-pyrrolidine-1-carboxylic acid [1-(S)-(3,5-bis-trifluoromethyl-phenyl)-ethyl]-methyl-amide
2-(S)-(4-Fluoro-2-methyl-phenyl)-3-(R)-{[methyl-piperidin-4-yl)-amino]-methyl}-pyrrolidine-1-carboxylic acid [1-(S)-(3,5-bis-trifluoromethyl-phenyl)-ethyl]-methyl-amide
2-(S)-(4-Fluoro-2-methyl-phenyl)-3-(R)-(4-methyl-pierazin-1-ylmethyl)-pyrrolidine-1-carboxylic acid [1-(S)-(3,5-bis-trifluoromethyl-phenyl)-ethyl]-methyl-amide
3-(R)-(3-Dimethylamino-pyrrolidin-1-ylmethyl)-2-(S)-(4-fluoro-2-methyl-phenyl)-pyrrolidine-1-carboxylic acid [1-(S)-(3,5-bis-trifluoromethyl-phenyl)-ethyl]-methyl-amide
2-(R)-(4-Fluoro-2-mehtyl-pheynyl)-3-(S)-piperidin-1-ylmethyl-pyrrolidine-1-carboxylic acid [1-(R)-(3,5-bis-trifluoromethyl-phenyl)-ethyl]-methyl-amide
3-(S)-Dimethylaminomethyl-2-(R)-(4-fluoro-2-methyl-phenyl)-pyrrolidine-1-carboxylic acid [1(R)-(3,5-bis-trufluoromethyl-phenyl)-ethyl]-methyl-amide
2-(S)-(4-Fluror-2-methyl-phenyl)-3-(R)-methylaminomethyl-pyrrolidine-1-carboxylic acid [1-(R)-(3,5-bis-trufluoromethyl-phenyl)-ethyl]-methyl-amide
2-(R)-(4-Fluoro-2-methyl-phenyl)-3-(S)-methylaminomethyl-pyrrolidine-1-carboxylic acid [1-(R)-(3,5-bis-trifluoromethyl-phenyl)-ethyl]-methyl-amide
2-(R)-(4-Fluoro-2-methyl-phenyl)-3-(S)-pyrrolidin-1-ylmethyl-pyrrolidine-1-carboxylic acid [1-(R)-(3,5-bis-trifluoromethyl-phenyl)-ethyl]-methyl-amide
2-(R)-(4-Fluoro-2-methyl-phenyl)-3-(S)-(4-methyl-piperidin-1-ylmethyl)-pyrrolidine-1-carboxylic acid [1-(R)-(3,5-bis-trifluoromethyl-phenyl)-ethyl]-methyl-amide
2-(R)-(4-Fluoro-2-methyl-phenyl)-3-(S)-(isopropylamino-methyl)-pyrrolidine-1-carboxylic acid [1-(R)-(3,5-bis-trifluoromethyl-phenyl)-ethyl]-methyl-amide
2-(R)-(4-Fluoro-2-methyl-phenyl)-3-(S)-piperazin-1-ylmethyl-pyrrolidine-1-carboxylic acid [1-(R)-(3,5-bis-trifluoromethyl-phenyl)-ethyl]-methyl-amide
2-(R)-(4-Fluoro-2-methyl-phenyl)-3-(S)-[(3-methylamino-propylamino)-methyl]-pyrrolidine-1-carboxylic acid [1-(R)-(3,5-bis-trifluoromethyl-phenyl)-ethyl]-methyl-amide
3-(S)-Azetidin-1-ylmethyl-2-(R)-(4-fluoro-2-methyl-phenyl)-pyrrolidine-1-carboxylic acid [1-(R)-(3,5-bis-trifluoromethyl-phenyl)-ethyl]-methyl-amide
3-(S)-[(Ethyl-methyl-amino)-methyl]-2-(R)-(4-fluoro-2-methyl-phenyl)-pyrrolidine-1-carboxylic acid [1-(R)-(3,5-bis-trifluoromethyl-phenyl)-ethyl]-methyl-amide
2-(R)-(4-Fluoro-2-methyl-phenyl)-3-(S)-(3-oxo-piperazin-1-ylmethyl)-pyrrolidine-1-carboxylic acid [1-(R)-(3,5-bis-trifluoromethyl-phenyl)-ethyl]-methyl-amide
2-(R)-(4-Fluoro-2-methyl-phenyl)-3-(S)-[(2-morpholin-4-yl-ethylamino)-methyl]-pyrrolidine-1-carboxylic acid [1-(R)-(3,5-bis-trifluoromethyl-phenyl)-ethyl]-methyl-amide
2-(R)-(4-Fluoro-2-methyl-phenyl)-3-(S)-[(2,2,2-trifluoro-ethylamino)-methyl]-pyrrolidine-1-carboxylic acid [1-(R)-(3,5-bis-trifluoromethyl-phenyl)-ethyl]-methyl-amide
3-(S)-[(2-Dimethylamino-ethylamino)-methyl]-2-(R)-(4-fluoro-2-methyl-phenyl)-pyrrolidine-1-carboxylic acid [1-(R)-(3,5-bis-trifluoromethyl-phenyl)-ethyl]-methyl-amide
2-(R)-(4-Fluoro-2-methyl-phenyl)-3-(S)-[(3-methoxy-propylamino)-methyl]-pyrrolidine-1-carboxylic acid [1-(R)-(3,5-bis-trifluoromethyl-phenyl)-ethyl]-methyl-amide
3-(S)-Cyclobutylaminomethyl-2-(R)-(4-fluoro-2-methyl-phenyl)-pyrrolidine-1-carboxylic acid [1-(R)-(3,5-bis-trifluoromethyl-phenyl)-ethyl]-methyl-amide
2-(R)-(4-Fluoro-2-methyl-phenyl)-3-(S)-{[3-(2-oxo-pyrrolidin-1-yl)-propylamino]-methyl}-pyrrolidine-1-carboxylic acid [1-(R)-(3,5-bis-trifluoromethyl-phenyl)-ethyl]-methyl-amide
3-(S)-(3-Ethoxy-propylamino)-methyl]-2-(R)-(4-fluoro-2-methyl-phenyl)-pyrrolidine-1-carboxylic acid [1-(R)-(3,5-bis-trifluoromethyl-phenyl)-ethyl]-methyl-amide
2-(R)-(4-Fluoro-2-methyl-phenyl)-3-(S)-[(2-hydroxy-1-methyl-ethylamino)-methyl]-pyrrolidine-1-carboxylic acid [1-(R)-(3,5-bis-trifluoromethyl-phenyl)-ethyl]-methyl-amide
2-(R)-(4-Fluoro-2-methyl-phenyl)-3-(S)-[(1-hydroxymethyl-propylamino)-methyl]-pyrrolidine-1-carboxylic acid [1-(R)-(3,5-bis-trifluoromethyl-phenyl)-ethyl]-methyl-amide
3-(S)-[(Cyclopropylmethyl-amino)-methyl]-2-(R)-(4-fluoro-2-methyl-phenyl)-pyrrolidine-1-carboxylic acid [1-(R)-(3,5-bis-trifluoromethyl-phenyl)-ethyl]-methyl-amide
2-o-Tolyl-pyrrolidine-1-carboxylic acid [1-(3,5-bis-trifluoromethyl-phenyl)-ethyl]-methyl-amide
3-Amino-2-(4-fluoro-2-methyl-phenyl)-piperidine-1-carboxylic acid [1-(3,5-bis-trifluoromethyl-phenyl)-ethyl]-methyl-amide (more polar isomer)
2-(4-Fluoro-2-methyl-phenyl)-3-methylamino-piperidine-1-carboxylic acid [1-(3,5-bis-trifluoromethyl-phenyl)-ethyl]-methyl-amide
3-Dimethylamino-2-(4-fluoro-2-methyl-phenyl)-piperidine-1-carboxylic acid [1-(3,5-bis-trifluoromethyl-phenyl)-ethyl]-methyl-amide
2-(4-Fluoro-2-methyl-phenyl)-3-methylamino-piperidine-1-carboxylic acid [1-(3,5-bis-trifluoromethyl-phenyl)-ethyl]-methyl-amide
2-(4-Fluoro-2-methyl-phenyl)-3-methylamino-piperidine-1-carboxylic acid [1-(3,5-bis-trifluoromethyl-phenyl)-ethyl]-methyl-amide
3-Dimethylamino-2-(4-fluoro-2-methyl-phenyl)-piperidine-1-carboxylic acid [1-(3,5-bis-trifluoromethyl-phenyl)-ethyl]-methyl-amide
2-(R)-(4-Fluoro-2-methyl-phenyl)-3-(S)-methylaminomethyl-piperidine-1-(R)-carboxylic acid [1-(3,5-bis-trifluoromethyl-phenyl)-ethyl]-methyl-amide
3-(S)-Dimethylaminomethyl-2-(R)-(4-fluoro-2-methyl-phenyl)-piperidine-1-(R)-carboxylic acid [1-(3,5-bis-trifluoromethyl-phenyl)-ethyl]-methyl-amide
Cis-3-Amino-2-phenyl-piperidine-1-carboxylic acid [1-(3,5-bis-trifluoromethyl-phenyl)-ethyl]-methyl-amide (less polar isomer)
Cis-3-Amino-2-phenyl-piperidine-1-carboxylic acid [1-(3,5-bis-trifluoromethyl-phenyl)-ethyl]-methyl-amide
{1-[(3,5-Bis-trifluoromethyl-benzyl)-methyl-carbamoyl]-2-(R)-phenyl-piperidin-3-(R)-yl}-methyl-carbamic acid tert-butyl ester
3-(R)-Methylamino-2-(R)-phenyl-piperidine-1-carboxylic acid (3,5-bis-trifluoromethyl-benzyl)-methyl-amide
3-(R)-Dimethylamino-2-(R)-phenyl-piperidine-1-carboxylic acid (3,5-bis-trifluoromethyl-benzyl)-methyl-amide
(1-{[1-(3,5-Bis-trifluoromethyl-phenyl)-ethyl]-methyl-carbamoyl}-2-(R)-phenyl-piperidin-3-(R)-yl)-methyl-carbamic acid tert-butyl ester (less polar)
(1-{[1-(3,5-Bis-trifluoromethyl-phenyl)-ethyl]-methyl-carbamoyl}-2-(R)-phenyl-piperidin-3-(R)-yl)-methyl-carbamic acid tert-butyl ester
3-(S)-Amino-2-(S)-phenyl-piperidine-1-carboxylic acid [1-(3,5-bis-trifluoromethyl-phenyl)-ethyl]-methyl-amide
3-(S)-Methylamino-2-(S)-phenyl-piperidine-1-carboxylic acid [1-(3,5-bis-trifluoromethyl-phenyl)-ethyl]-methyl-amide
3-(S)-Methylamino-2-(S)-phenyl-piperidine-1-carboxylic acid [1-(3,5-bis-trifluoromethyl-phenyl)-ethyl]-methyl-amide
3-(S)-Amino-2-(R)-phenyl-piperidine-1-carboxylic acid [1-(3,5-bis-trifluoromethyl-phenyl)-ethyl]-methyl-amide
3-(R)-Methylamino-2-(S)-phenyl-piperidine-1-carboxylic acid [1-(3,5-bis-trifluoromethyl-phenyl)-ethyl]-methyl-amide
3-(R)-Methylamino-2-(S)-phenyl-piperidine-1-carboxylic acid [1-(3,5-bis-trifluoromethyl-phenyl)-ethyl]-methyl-amide
3-(R)-Amino-2-(S)-o-tolyl-piperidine-1-carboxylic acid [1-(R)-(3,5-bis-trifluoromethyl-phenyl)-ethyl]-methyl-amide
(1-(R)-{[1-(3,5-Bis-trifluoromethyl-phenyl)-ethyl]-methyl-carbamoyl}-2-(S)-o-tolyl-piperidin-3-(R)-yl)-methyl-carbamic acid tert-butyl ester
3-(R)-Pyrrolidin-1-yl-2-(S)-o-tolyl-piperidine-1-carboxylic acid [1-(R)-(3,5-bis-trifluoromethyl-phenyl)-ethyl]-methyl-amide
3-(R)-Dimethylamino-2-(S)-o-tolyl-piperidine-1-carboxylic acid [1-(R)-(3,5-bis-trifluoromethyl-phenyl)-ethyl]-methyl-amide
(1-(R)-{[1-(3,5-Bis-trifluoromethyl-phenyl)-ethyl]-methyl-carbamoyl}-2-(S)-o-tolyl-pyrrolidin-3-(R)-yl)-carbamic acid tert-butyl ester
3-(R)-Amino-2-(S)-o-tolyl-pyrrolidine-1-(R)-carboxylic acid [1-(3,5-bis-trifluoromethyl-phenyl)-ethyl]-methyl-amide
3-(R)-Methylamino-2-(S)-o-tolyl-pyrrolidine-1-carboxylic acid [1-(R)-(3,5-bis-trifluoromethyl-phenyl)-ethyl]-methyl-amide
1-(4-Fluoro-2-methyl-phenyl)-hexahydro-pyrrolo[3,4-c]pyrrole-2-carboxylic acid [1-(3,5-bis-trifluoromethyl-phenyl)-ethyl]-methyl-amide
1-(2-methylphenyl)-hexahydro-pyrrolo[3,4-c]pyrrole-2-carboxylic acid [1-(3,5-bis-trifluoromethyl-phenyl)-ethyl]-methyl-amide
1-Phenyl-hexahydro-pyrrolo[3,4-c]pyrrole-2-carboxylic acid [1-(3,5-bis-trifluoromethyl-phenyl)-ethyl]-methyl-amide
1-(4-Fluoro-2-methyl-phenyl)-5-(pyrrolidin-1-yl-acetyl)-hexahydro-pyrrolo[3,4-c]pyrrole-2-carboxylic acid [1-(3,5-bis-trifluoromethyl-phenyl)-ethyl]-methyl-amide
5-Benzyl-1-(4-fluoro-2-methyl-phenyl)-hexahydro-pyrrolo[3,4-c]pyrrole-2-carboxylic acid [1-(3,5-bis-trifluoromethyl-phenyl)-ethyl]-methyl-amide
5-Benzyl-1-(2-methylphenyl)-hexahydro-pyrrolo[3,4-c]pyrrole-2-carboxylic acid [1-(3,5-bis-trifluoromethyl-phenyl)-ethyl]-methyl-amide
5-Benzyl-1-phenyl-hexahydro-pyrrolo[3,4-c]pyrrole-2-carboxylic acid [1-(3,5-bis-trifluoromethyl-phenyl)-ethyl]-methyl-amide
2-(4-Fluoro-2-methyl-phenyl)-3,7-diaza-bicyclo[3.3.1]nonane-3-carboxylic acid [1-(3,5-bis-trifluoromethyl-phenyl)-ethyl]-methyl-amide
2-(2-methyl-phenyl)-3,7-diaza-bicyclo[3.3.1]nonane-3-carboxylic acid [1-(3,5-bis-trifluoro methyl-phenyl)-ethyl]-methyl-amide
2-phenyl-3,7-diaza-bicyclo[3.3.1]nonane-3-carboxylic acid [1-(3,5-bis-trifluoro methyl-phenyl)-ethyl]-methyl-amide
7-Benzyl-2-(4-fluoro-2-methyl-phenyl)-3,7-diaza-bicyclo[3.3.1]nonane-3-carboxylic acid [1-(3,5-bis-trifluoromethyl-phenyl)-ethyl]-methyl-amide
7-Benzyl-2-(2-methyl-phenyl)-3,7-diaza-bicyclo[3.3.1]nonane-3-carboxylic acid [1-(3,5-bis-trifluoromethyl-phenyl)-ethyl]-methyl-amide
7-Benzyl-2-phenyl-3,7-diaza-bicyclo[3.3.1]nonane-3-carboxylic acid [1-(3,5-bis-trifluoromethyl-phenyl)-ethyl]-methyl-amide
1-{4-[2-Benzyl-1-(3,5-bis-trifluoromethyl-benzoyl)-piperidin-3-ylamino]-piperidin-1-yl}-ethanone
1-{4-[2-Benzyl-1-(3,5-bis-trifluoromethyl-benzoyl)-piperidin-3-ylmethyl]-piperazin-1-yl}-ethanone

14. A pharmaceutical composition comprising the compound of claim 1 or a pharmaceutically acceptable salt thereof; and a pharmaceutically acceptable carrier.

15. The pharmaceutical composition of claim 14 wherein said composition is formulated for oral or injectable administration.

16. The pharmaceutical composition of claim 15 wherein said composition is an immediate release or a controlled release dosage form.

17. The compound of claim 1 wherein in an assay of NK-1 binding, said compound exhibits a Ki of about 1 uM or less.

18. The compound of claim 17 wherein said Ki is about 10 nM or less.

19. A method of treating a mammal for conditions mediated by neurokinins which comprises administering to a mammal in need of such treatment a therapeutically effective amount of the compound of claim 1.
